(19) United States
(12) Patent Application Publication
(10) Pub. No.: US 2022/0186256 A1
(43) Pub. Date: Jun. 16, 2022

(54) RECOMBINANT ADENO-ASSOCIATED VIRUSES AND USES THEREOF

(71) Applicant: REGENXBIO INC., Rockville, MD (US)

(72) Inventors: Olivier Danos, Princeton, NJ (US); Ye Liu, Clarksville, MD (US); Andrew Mercer, Poolesville, MD (US); Samantha Yost, Rockville, MD (US); Subha Karumuthil-Melethil, Germantown, MD (US); Elad Firnberg, Baltimore, MD (US)

(21) Appl. No.: 17/600,418

(22) PCT Filed: Apr. 2, 2020
(86) PCT No.: PCT/US20/26485

§ 371 Date: Sep. 30, 2021

Related U.S. Application Data
(60) Provisional application No. 62/829,608, filed on Apr. 4, 2019.
(60) Provisional application No. 62/833,516, filed on Apr. 12, 2019.
(60) Provisional application No. 62/839,368, filed on Apr. 26, 2019.
(60) Provisional application No. 62/924,107, filed on Oct. 21, 2019.
(60) Provisional application No. 62/963,512, filed on Jan. 20, 2020.

(51) Int. Cl. C12N 15/86; C07K 14/005

(52) U.S. Cl. CPC C12N 15/86; C07K 14/005; C12N 2750/14145; C12N 2750/14143; C12N 2750/14122

(57) ABSTRACT

The present invention relates to recombinant adeno-associated viruses (rAAVs) having capsid proteins engineered to include amino acid sequences that confer and/or enhance desired properties. In particular, the invention provides engineered capsid proteins comprising peptide insertions from heterologous proteins inserted within or near variable region IV (VR-IV) of the virus capsid, such that the insertion is surface exposed on the AAV particle. The invention also provides capsid proteins that direct rAAVs to target tissues, in particular, capsid proteins comprising peptides derived from erythropoietin or dynein that are inserted into surface-exposed variable regions and that target rAAVs to retinal tissue and/or neural tissue, including the central nervous system, and deliver therapeutics for treating neurological and/or eye disorders.

### 0. SEQUENCE LISTING

The instant application contains a Sequence Listing which has been submitted electronically in ASCII format and is hereby incorporated by reference in its entirety. Said ASCII copy, created on Mar. 29, 2020, is named 38013_0002P1_SL.txt and is 637,866 bytes in size.

### 1. FIELD OF THE INVENTION

The present invention relates to recombinant adeno-associated viruses (rAAVs) having capsid proteins engineered to include amino acid sequences that confer and/or enhance desired properties. In particular, the invention provides engineered capsid proteins comprising peptide insertions from heterologous proteins inserted within or near variable region IV (VR-IV) or, alternatively, within or near variable region VIII (VR-VIII) of the virus capsid, such that the insertion is surface exposed on the AAV particle. The invention also provides capsid proteins that direct rAAVs to target tissues, in particular, capsid proteins comprising peptides derived from e.g. erythropoietin or dynein inserted into surface-exposed variable regions to target rAAVs to and/or improve transduction of retinal and neural tissue, including the central nervous system, and deliver therapeutics for treating neurological disorders.

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and over twenty gene therapy products are currently in clinical development. Recombinant AAVs, such as AAV9, have demonstrated desirable neurotropic properties and clinical trials using recombinant AAV9 for treatment of CNS disease are underway. However, attempts to enhance the neurotropic properties of rAAVs in human subjects have met with limited success.

There remains a need for rAAV vectors with enhanced neurotropic properties for use, e.g., in crossing the blood brain barrier to delivery therapies in treating disorders associated with the central nervous system and the eye, particularly the retina. There also is a need for rAAV vectors with enhanced tissue-specific targeting and/or enhanced tissue-specific transduction to deliver therapies.

### 3. SUMMARY OF THE INVENTION

Provided are recombinant adeno-associated viruses (rAAVs) having capsid proteins engineered to include amino acid sequences that confer and/or enhance desired properties such as tissue targeting, transduction and/or integration of the rAAV genome. In particular, the invention provides engineered capsid proteins comprising one or more peptide insertions from heterologous proteins inserted within or near variable region IV (VR-IV) of the virus capsid, or within or near variable region VIII (VR-VIII), such that the insertion is surface exposed on the AAV particle. In particular embodiments, the insertion is immediately after an amino acid residue corresponding to one of the amino acids 451 to 461 of the AAV9 capsid protein (SEQ ID NO:118 and as numbered in FIG. 8), including after the amino acid 454 (i.e., between amino acid 454 and 455) of the AAV9 capsid or in a capsid protein of a different AAV type after a residue that corresponds to the amino acid 454 of AAV9 (e.g., SEQ ID NO: 110-117 or 119-121) “corresponding to” meaning aligned with in the sequence alignment in FIG. 8 or for AAV types not included in FIG. 8, a similar amino acid sequence alignment of the AAV9 capsid protein sequence (SEQ ID NO:118) and the AAV capsid protein as would be well known in the art). Thus, the invention provides an engineered capsid protein comprising a peptide insertion from a heterologous protein inserted immediately after or near an amino acid corresponding to the amino acid residue at position 454 of AAV9, as numbered in FIG. 8. In additional particular embodiments, the insertion is immediately after an amino acid residue corresponding to amino acid 588 (i.e., between amino acids 588 and 589) of the AAV9 capsid protein (SEQ ID NO:118 and as numbered in FIG. 8), or in a capsid protein of a different AAV type after a residue that corresponds to the amino acid 588 of AAV9 (e.g., SEQ ID NO: 110-117 or 119-121). The capsid protein may be an AAV9 capsid protein but may also be any AAV capsid protein, such as AAV serotype 1 (SEQ ID NO: 110); AAV serotype 2 (SEQ ID NO: 111); AAV serotype 3 (SEQ ID NO: 112) AAV serotype 4 (SEQ ID NO: 113); AAV serotype 5 (SEQ ID NO: 114); AAV serotype 6 (SEQ ID NO: 115); 451-461 of AAV7 capsid (SEQ ID NO: 116); 451-461 of AAV8 capsid (SEQ ID NO: 117); AAV serotype 9 (SEQ ID NO: 118); AAV serotype 9e (SEQ ID NO: 119); AAV serotype rh10 (SEQ ID NO: 120); AAV serotype rh20 (SEQ ID NO: 121); and AAV serotype hu.37 (SEQ ID NO: 122), AAV serotype rh39 (SEQ ID NO: 124), and AAV serotype rh74 (SEQ ID NO: 123 or SEQ ID NO: 154) (see FIG. 8).

Also provided are capsid proteins that direct rAAVs to target tissues, in particular, capsid proteins comprising peptides derived from erythropoietin or dynein (including axonemal or cytoplasmic dynein) or a peptide that promotes tissue targeting and/or cellular uptake and/or integration of the rAAV genome, that are inserted into surface-exposed variable regions and that target rAAVs to neural tissue, including to the central nervous system, and to retinal tissue, and deliver therapeutics for treating neurological and ocular disorders. These peptides are advantageously inserted into the amino acid sequence of the capsid protein such that, when the capsid protein is incorporated into the AAV particle, the inserted peptide is surface exposed. These peptides are inserted immediately after one of the amino acids of, or after one of the amino acids corresponding to the amino acid, 262-273; 451-461; or 585-593 of AAV9 capsid (SEQ ID NO:118 and see FIG. 8 for alignment), or immediately after an amino acid residue corresponding to the first codon encoding VP2, that is amino acid 138 of the AAV9 capsid and amino acids corresponding to position 138 of the AAV9 capsid (SEQ ID NO:118 and see FIG. 8 for alignment). In certain embodiments the inserted peptide is at least 4 contiguous amino acids or is 5, 6, 7 contiguous amino acids of one of the peptides KMQVPFQ (SEQ ID NO: 1); TLAAPFK (SEQ ID NO: 2); QQAAPSF (SEQ ID NO: 3); RYNAPFK (SEQ ID NO: 4); LKLPPIV (SEQ ID NO: 5); PFIKPFE (SEQ ID NO: 6); or TLSLPWK (SEQ ID NO: 7) of the axonemal dynein heavy chain or is alternatively 5, 6, 7, 8, 9, 10 or 11 contiguous amino acids of QEQLERALNS S (SEQ ID NO: 8), which is a non-linear epitope of erythropoietin called ARA290.

Provided are engineered capsid proteins comprising peptides that target specific tissues, including to promote or increase cellular uptake and/or integration of an rAAV genome, wherein the peptides are inserted into surface-exposed variable regions of the capside protein. In certain embodiments, the peptides target and/or promote transduction or genome integration in cells of bone (for example, at least 4 contiguous amino acids or at least 7 or 8 contiguous amino acids of DDDDDDDD (SEQ ID NO: 9)), brain (at least 4 amino acids or at least 7 contiguous amino acids or is 7 contiguous amino acids of LSSRLDA (SEQ ID NO: 10) or is 7, 8 or 9 contiguous amino acids of CLSSRLDAC (SEQ ID NO: 11)), kidney (at least 4 or 5 contiguous amino acids of or is the peptide CLPVASC (SEQ ID NO: 12) or LPVAS (SEQ ID NO: 13)), muscle (at least 4, 5, 6, or 7 contiguous amino acids or is the peptide of ASSLNIA (SEQ ID NO: 14)), retinal cells (at least 4 contiguous amino acids of or is 5, 6, or 7 contiguous amino acids of LGETTRP (SEQ ID NO: 15) or LALGETTRP (SEQ ID NO: 16)), or is derived from the transferrin receptor (at least 4 contiguous amino acids of or at least 7 contiguous amino acids of or is 7 contiguous amino acids of HAIYPRH (SEQ ID NO: 17), THRPPMWSPVWP (SEQ ID NO: 18), RTIGPSV (SEQ ID NO: 19), or CRTIGPSVC (SEQ ID NO: 20)). In certain embodiments, the peptide is CLPVASC (SEQ ID NO: 12) or is ASSLNIA (SEQ ID NO: 14) and capsids containing this peptide, for example, inserted after position 454 of AAV9, preferentially target the rAAV with the capsid to the kidney as compared to the liver. In other embodiments, the inserted peptide is at least 4 contiguous amino acids or at least 7 or 8 contiguous amino acids or is the peptide SITLVKSTQTV (SEQ ID NO: 21) or TILSRSTQTG (SEQ ID NO: 22) or QAVRTSL (SEQ ID NO: 23) or QAVRTSH (SEQ ID NO: 24). In some embodiments, the peptide is no more than 12 contiguous amino acids. In other embodiments, provided are engineered capsids having one or more amino acid substitutions which may improve tropism, transduction or reduce immune neutralizing activity. Such amino acid modifications include A269S of AAV8, and corresponding substitutions in other AAV type capsids, S263F/S269T/A273T of AAV9, and corresponding substitutions in other AAV type capsids, W530R or Q474A of AAV9, and corresponding substitutions in other AAV type capsids. The capsids having these amino acid substitutions may further have substitution of the NNN (asparagines) at 498 to 500 with AAA (alanines) of the AAV8 capsid, or substitutions of the NNN (asparagines) at 496 to 498 with AAA (alanines) of the AAV9 capsid, or corresponding substitutions in other AAV type capsids.

Also provided are engineered capsid proteins that promote transduction of the rAAV in one or more tissues, including one or more cell types, upon systemic, intravenous, intrathecal, intranasal, intraperitoneal, or intravitreal administration, wherein the capsid proteins comprise a peptide that is inserted into a surface-exposed variable region (VR) of the capsid, e.g. VR-I, VR-IV or VR-VIII, or after the first amino acid of VP2, e.g., immediately after residue 138 of the AAV9 capsid (amino acid sequence of SEQ ID NO:118) or immediately after the corresponding residue of another AAV capsid, or alternatively is engineered with one or more of the amino acid substitutions described herein, and transduction of the AAV having the engineered capsid in the at least one tissue is increased upon said administration compared to the transduction of the AAV having the corresponding unengineered capsid. In certain embodiments, transduction is measured by detection of transgene, such as GFP fluorescence.

In certain embodiments, provided are rAAVs incorporating the engineered capsids described herein, including rAAVs with genomes comprising a transgene of therapeutic interest. Packaging cells for producing the rAAVs described herein are provided. Method of treatment by delivery of, and pharmaceutical compositions comprising, the engineered rAAVs described herein are also provided. Also provided are methods of manufacturing the rAAVs with the engineered capsids described herein.

The invention is illustrated by way of examples infra describing the construction of rAAV9 capsids engineered with peptide inserts designed on the basis of the human axonemal dynein heavy chain tail, ARA290, and other tissue targeting or homing peptides and capsids engineered with amino acid substitutions.

### 3.1. Embodiments

1. A recombinant adeno-associated virus (rAAV) capsid protein comprising a peptide insertion of at least 4 and up to 20 contiguous amino acids from a heterologous protein that is not a capsid protein, said peptide insertion being immediately after an amino acid residue corresponding to one of amino acids 451 to 461 of AAV9 capsid protein of FIG. 8 (SEQ ID NO:118), wherein said peptide insertion is surface exposed when said capsid protein is packaged as an AAV particle.

2. The rAAV capsid protein of embodiment 1, wherein said capsid protein is from at least one AAV serotype of AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37) or serotype rh74 (AAVrh74).

3. The rAAV capsid protein of embodiment 2, wherein said peptide insertion occurs immediately after one of the amino acid residues within:

450-459 of AAV1 capsid amino acid sequence (SEQ ID NO 110);

449-458 of AAV2 capsid amino acid sequence (SEQ ID NO. 111);

449-459 of AAV3 capsid amino acid sequence (SEQ ID NO, 112);

443-453 of AAV4 capsid amino acid sequence (SEQ ID NO. 113);

442-445 of AAV5 capsid amino acid sequence (SEQ ID NO. 114);

450-459 of AAV6 capsid amino acid sequence (SEQ ID NO. 115);

451-461 of AAV7 capsid amino acid sequence (SEQ ID NO. 116);

451-461 of AAV8 capsid amino acid sequence (SEQ ID NO. 117);

451-461 of AAV9 capsid amino acid sequence (SEQ ID NO. 118);

452-461 of AAV9e capsid amino acid sequence (SEQ ID NO. 119);

452-461 of AAVrh10 capsid amino acid sequence (SEQ ID NO. 120);

452-461 of AAVrh20 capsid amino acid sequence (SEQ ID NO. 121);

452-461 of AAVhu.37 capsid amino acid sequence (SEQ ID NO. 122);

452-461 of AAVrh74 capsid amino acid sequence (SEQ ID NO 123 or SEQ ID NO: 154); or

452-461 of AAVrh39 capsid amino acid sequence (SEQ ID NO. 124) in the sequences depicted in FIG. 8.

4. The rAAV capsid protein of embodiment 3, wherein said peptide insertion occurs immediately after one of the amino acid residues I451, N452, G453, S454, G455, Q456, N457, Q458, Q459, T460, or L461 of AAV9 capsid or the immediately after the amino acid residue in an AAV capsid corresponding to amino acid I451, N452, G453, S454, G455, Q456, N457, Q458, Q459, T460, or L461 of AAV9 capsid (SEQ ID NO:118) as aligned and according to the amino acid numbering of FIG. 8.

5. The rAAV capsid protein of any preceding embodiment, wherein said heterologous protein is a homing domain, a neutralizing antibody epitope, or a purification tag.

6. The rAAV capsid protein of embodiment 5, wherein said homing domain is

a neural tissue-homing domain;

an axonemal or cytoplasmic dynein-homing domain;

a bone-homing domain;

a kidney-homing domain;

a muscle-homing domain;

an endothelial cell-homing domain;

an integrin receptor-binding domain;

a transferrin receptor-binding domain;

a tumor cell-targeting domain; or

a retinal cell homing domain.

7. The rAAV capsid protein of embodiment 6, wherein the peptide insertion comprises or consists of a dynein peptide or dynein-homing peptide of at least 4 or at least 7 contiguous amino acids of amino acid sequence SITLVKSTQTV (SEQ ID NO: 21), TILSRSTQTG (SEQ ID NO: 22), VVMVGEKPITITQHSVETEG (SEQ ID NO: 25), RSSEEDKSTQTT (SEQ ID NO: 26), KMQVPFQ (SEQ ID NO: 1), LKLPPIV (SEQ ID NO: 5), PFIKPFE (SEQ ID NO: 6), TLSLPWK (SEQ ID NO: 7), QQAAPSF (SEQ ID NO: 3), RYNAPFK (SEQ ID NO: 4), TLAVPFK (SEQ ID NO: 27), TLAAPFK (SEQ ID NO: 2), LGETTRP (SEQ ID NO: 15), or LALGETTRP (SEQ ID NO: 16).

8. The rAAV capsid protein of embodiment 6, wherein the peptide insertion from said transferrin receptor-binding domain comprises at least 4 or at least 7 contiguous amino acids of amino acid sequence RTIGPSV (SEQ ID NO: 19) or CRTIGPSVC (SEQ ID NO: 20).

9. The rAAV capsid protein of embodiment 6, wherein the peptide insertion from said retinal cell-homing domain comprises amino acid sequence LGETTRP (SEQ ID NO: 15) or LALGETTRP (SEQ ID NO: 16).

10. The rAAV capsid protein of embodiment 9, wherein the LGETTRP (SEQ ID NO: 15) or LALGETTRP (SEQ ID NO: 16) peptide insertion occurs in the AAV8 capsid protein or in the AAV9 capsid protein.

11. The rAAV capsid protein of any of the previous embodiments, wherein the peptide insertion occurs in an AAV9 capsid protein after amino acid S454 of the AAV9 capsid protein (SEQ ID NO:118) or in an AAV capsid protein after a residue corresponding to S454 of the AAV9 capsid protein as aligned and according to the amino acid numbering in FIG. 8.

12. The rAAV capsid protein of any of the above embodiments, with the proviso that the capsid protein is not the AAV2 capsid protein.

13. A nucleic acid comprising a nucleotide sequence encoding the rAAV capsid protein of any of the above embodiments, or encoding an amino acid sequence sharing at least 80% identity therewith.

14. A packaging cell capable of expressing the nucleic acid of embodiment 13 to produce AAV vectors comprising the capsid protein encoded by said nucleotide sequence.

15. A rAAV vector comprising the capsid protein of any of embodiments 1-12.

16. The rAAV vector of embodiment 15, further comprising a transgene.

17. A pharmaceutical composition comprising the rAAV vector of embodiment 15 or 16 and a pharmaceutically acceptable carrier.

18. A method of delivering a transgene to a cell, said method comprising contacting said cell with the rAAV vector of embodiment 16, wherein said transgene is delivered to said cell.

19. A method of delivering a transgene to a target tissue, or a target cell or cellular matrix thereof, of a subject in need thereof, said method comprising administering to said subject the rAAV vector of embodiment 16, wherein the transgene is delivered to the target tissue of said subject.

20. A pharmaceutical composition for use in delivering a transgene to a cell, said composition comprising the rAAV vector of embodiment 16 wherein said cell is contacted with the vector.

21. A pharmaceutical composition for use in delivering a transgene to a target tissue of a subject in need thereof, said pharmaceutical composition comprising the rAAV vector of embodiment 16, wherein said peptide insertion is a homing peptide and wherein the vector is administered to said subject.

22. The method, or pharmaceutical composition for use, according to embodiments 18 to 21, wherein said rAAV vector is administered systemically, intravenously, intrathecally, intra-nasally, intra-peritoneally, or intravitreally.

23. The method, or pharmaceutical composition for use, according to embodiments 18 to 21, wherein said target tissue, or a target cell or cellular matrix thereof, is:
a neural tissue, and said vector comprises the peptide insertion from said neural tissue-homing domain;
bone, and said vector comprises the peptide insertion from said bone-homing domain;
kidney, and said vector comprises the peptide insertion from said kidney-homing domain;
muscle, and said vector comprises the peptide insertion from said muscle-homing domain;
an endothelial cell, and said vector comprises the peptide insertion from said endothelial cell-homing domain;
a cell expressing an integrin receptor, and said vector comprises the peptide insertion from said integrin receptor-binding domain;
a tumor cell expressing a transferrin receptor, and said vector comprises the peptide insertion from said transferrin receptor-binding domain; and
a tumor cell, and said vector comprises the peptide insertion from said tumor cell-targeting domain; or
a retinal cell, and said vector comprises the peptide insertion from said retinal cell-homing domain.

24. A recombinant adeno-associated virus (rAAV) capsid protein, said capsid protein comprising a peptide insertion of at least 4 and up to 20 contiguous amino acids from a heterologous protein or domain selected from the group consisting of
a neural tissue-homing protein or domain, with the proviso that the peptide insertion does not comprise sequence TLAVPFK (SEQ ID NO: 27);
an axonemal or cytoplasmic dynein-homing domain;
a bone-homing domain;
a kidney-homing domain;
a muscle-homing domain;
an endothelial cell-homing domain;
an integrin receptor-binding domain;
a transferrin receptor-binding domain, with the proviso that the peptide insertion does not comprise sequence RTIGPSV (SEQ ID NO: 19) nor CRTIGPSVC (SEQ ID NO: 20);
a tumor cell-targeting domain; and
a retinal cell-homing domain, with the proviso that the peptide insertion does not comprise sequence LGETTRP (SEQ ID NO: 15) nor LALGETTRP (SEQ ID NO: 16).
wherein said peptide insertion is surface exposed when said capsid protein is packaged as an AAV particle.

25. The rAAV capsid protein of embodiment 24, wherein said neural tissue-homing protein or retinal cell-homing domain is a human axonemal dynein (HAD) heavy chain tail.

26. The rAAV capsid protein of embodiment 25, wherein said peptide insertion comprises at least 4 and up to 12 contiguous amino acids from a dimerization domain of said HAD heavy chain tail.

27. The rAAV capsid protein of embodiment 26, wherein said peptide insertion comprises at least 4 and up to 12 contiguous amino acids from the group consisting of (depicted in FIGS. 7A-7M):

(aa 1-1542 of DYH1_HUMAN UniProtKB-Q9P2D7) (SEQ ID NO. 97);

(aa 1-1764 of DYH2_HUMAN UniProtKB-Q9P225) (SEQ ID NO. 98);

(aa 1-1390 of DYH3_HUMAN UniProtKB-Q8TD57) (SEQ ID NO. 99);

(aa 1-1941 of DYH5_HUMAN UniProtKB-Q8TE73) (SEQ ID NO. 100);

(aa 1-1433 of DYH6_HUMAN UniProtKB-Q9C0G6) (SEQ ID NO. 101);

(aa 1-1289 of DYH7_HUMAN UniProtKB-Q8WXX0) (SEQ ID NO. 102);

(aa 1-1807 of DYH8_HUMAN UniProtKB-Q96JB1) (SEQ ID NO. 103);

(aa 1-1831 of DYH9_HUMAN UniProtKB-Q9NYC9) (SEQ ID NO. 104);

(aa 1-1793 of DYH10_HUMAN UniProtKB-Q8IVF4) (SEQ ID NO. 105);

(aa 1-1854 of DYH11_HUMAN UniProtKB-Q96DT5) (SEQ ID NO. 106);

(aa 1-1214 of DYH12_HUMAN UniProtKB-Q6ZR08) (SEQ ID NO. 107);

(aa 1-200 of DYH14_HUMAN UniProtKB-Q0VDD8) (SEQ ID NO. 108); or

(aa 1-1794 of DYH17_HUMAN UniProtKB-Q9UFH2) (SEQ ID NO. 109).

28. The rAAV capsid protein of embodiment 27, wherein said peptide insertion comprises at least 4 and up to 12 contiguous amino acids from residues 1-200 of any one of the axonemal dynein heavy chain sequences (FIGS. 7A-7M).

29. The rAAV capsid protein of embodiment 27, wherein said peptide insertion comprises 7 contiguous amino acids from any one of the dynein heavy chain sequences of FIG. 7A-7M.

30. The rAAV capsid protein of embodiment 28, wherein said peptide insertion comprises 7 contiguous amino acids from residues 1-200 of any one of the dynein heavy chain sequences (FIG. 7A-7M)

31. The rAAV capsid protein of embodiment 25, wherein said peptide insertion comprises at least 4 contiguous amino acids one of:

(SEQ ID NO: 1)
KMQVPFQ;
(SEQ ID NO: 2)
TLAAPFK;
(SEQ ID NO: 3)
QQAAPSF;
(SEQ ID NO: 4)
RYNAPFK;
(SEQ ID NO: 5)
LKLPPIV;
(SEQ ID NO: 6)
PFIKPFE;
or
(SEQ ID NO: 7)
TLSLPWK.

32. The rAAV capsid protein of embodiment 25, wherein said peptide insertion consists of a peptide from one of:

(SEQ ID NO: 1)
KMQVPFQ;
(SEQ ID NO: 2)
TLAAPFK;
(SEQ ID NO: 3)
QQAAPSF;
(SEQ ID NO: 4)
RYNAPFK;
(SEQ ID NO: 5)
LKLPPIV;
(SEQ ID NO: 6)
PFIKPFE;
or
(SEQ ID NO: 7)
TLSLPWK.

33. The rAAV capsid protein of embodiment 32, wherein said peptide insertion comprises the amino acid sequence TLAAPFK (SEQ ID NO: 2);

34. The rAAV capsid protein of embodiment 24, wherein said neural tissue-homing protein is a mouse axonemal dynein (MAD) heavy chain tail.

35. The rAAV capsid protein of embodiment 24, wherein said neural tissue-homing domain is an EPO (erythropoietin) domain that binds innate repair receptor and is not erythropoietic, or a conformational analog of said domain.

36. The rAAV capsid protein of embodiment 35, wherein the peptide insertion comprises at least 4 and up to 11 contiguous amino acids from QEQLERALNSS (SEQ ID NO: 8).

37. The rAAV capsid protein of embodiment 36, wherein said peptide insertion is the ARA290 sequence QEQLERALNSS (SEQ ID NO: 8).

38. The rAAV capsid protein of embodiment 24, wherein said neural tissue-homing protein is a brain-homing domain having an SRL (serine-arginine-lysine) motif

39. The rAAV capsid protein of embodiment 38, wherein the peptide insertion from said brain-homing domain comprises at least 7 contiguous amino acids of the amino acid sequence LSSRLDA (SEQ ID NO: 10) or CLSSRLDAC (SEQ ID NO: 11).

40. The rAAV capsid protein of embodiment 24, wherein said axonemal or cytoplasmic dynein-homing domain is a dynein light chain-homing domain.

41. The rAAV capsid protein of embodiment 40, wherein the peptide insertion from said dynein light chain-homing domain is one of SITLVKSTQTV (SEQ ID NO: 21), TILSRSTQTG (SEQ ID NO: 22), VVMVGEKPITITQHSVETEG (SEQ ID NO: 25), or RSSEEDKSTQTT (SEQ ID NO: 26).

42. The rAAV capsid protein of embodiment 24, wherein said bone-homing protein is a hydroxyapatite (HA)-binding domain.

43. The rAAV capsid protein of embodiment 42, wherein the peptide insertion from said hydroxyapatite (HA)-binding domain is at least 6 amino acid residues of the sequence DDDDDDDD (SEQ ID NO: 9).

44. The rAAV capsid protein of embodiment 24, wherein said kidney-homing domain is amino acid sequence CLPVASC (SEQ ID NO: 12).

45. The rAAV capsid protein of embodiment 44, wherein the peptide insertion from said kidney-homing domain is amino acid sequence LPVAS (SEQ ID NO: 13) or CLPVASC (SEQ ID NO: 12).

46. The rAAV capsid protein of embodiment 24, wherein the peptide insertion from said muscle-homing domain is amino acid sequence ASSLNIA (SEQ ID NO: 14).

47. The rAAV capsid protein of embodiment 24, wherein the peptide insertion is amino acid sequence QAVRTSL (SEQ ID NO: 23) or QAVRTSH (SEQ ID NO: 24).

48. The rAAV capsid protein of embodiment 24, wherein the peptide insertion from said endothelial cell-homing domain is the amino acid sequence SIGYPLP (SEQ ID NO: 28).

49. The rAAV capsid protein of embodiment 24, wherein the peptide insertion from said integrin-binding domain has amino acid sequence CDCRGDCFC (SEQ ID NO: 29).

50. The rAAV capsid protein of embodiment 24, wherein said transferrin receptor-binding domain is a transferrin domain, or a conformation analog thereof, or an iron-mimic.

51. The rAAV capsid protein of embodiment 50, wherein the peptide insertion from said transferrin domain comprises at least 4 contiguous amino acids and up to 12 contiguous amino acids from sequence HAIYPRH (SEQ ID NO: 17) or THRPPMWSPVWP (SEQ ID NO: 18).

52. The rAAV capsid protein of embodiment 51, wherein the peptide insertion is amino acid sequence HAIYPRH (SEQ ID NO: 17) or THRPPMWSPVWP (SEQ ID NO: 18).

53. The rAAV capsid protein of embodiment 24, wherein the peptide insertion from said tumor cell-targeting domain is amino acid sequence NGRAHA (SEQ ID NO: 30).

54. The rAAV capsid protein of any of embodiments 24-53, wherein said peptide insertion occurs immediately after one of the amino acid residues (as depicted in FIG. 8):
138; 262-272; 450-459; or 585-593 of AAV1 capsid amino acid sequence (SEQ ID NO. 110);
138; 262-272; 449-458; or 584-592 of AAV2 capsid; amino acid sequence (SEQ ID NO. 111)
138; 262-272; 449-459; or 585-593 of AAV3 capsid amino acid sequence (SEQ ID NO. 112);
137; 256-262; 443-453; or 583-591 of AAV4 capsid amino acid sequence (SEQ ID NO. 113);
137; 252-262; 442-445; or 574-582 of AAV5 capsid amino acid sequence (SEQ ID NO. 114);
138; 262-272; 450-459; 585-593 of AAV6 capsid amino acid sequence (SEQ ID NO. 115);
138; 263-273; 451-461; 586-594 of AAV7 capsid amino acid sequence (SEQ ID NO. 116);
138; 263-274; 452-461; 587-595 of AAV8 capsid amino acid sequence (SEQ ID NO. 117);
138; 262-273; 452-461; 585-593 of AAV9 capsid amino acid sequence (SEQ ID NO. 118);
138; 262-273; 452-461; 585-593 of AAV9e capsid amino acid sequence (SEQ ID NO. 119);
138; 263-274; 452-461; 587-595 of AAVrh10 capsid amino acid sequence (SEQ ID NO. 120);
138; 263-274; 452-461; 587-595 of AAVrh20 capsid amino acid sequence (SEQ ID NO. 121);
138; 263-274; 452-461; 587-595 of AAVhu37 capsid amino acid sequence (SEQ ID NO. 122)
138; 263-274; 452-461; 587-595 of AAVrh74 capsid amino acid sequence (SEQ ID NO. 123 or SEQ ID NO: 154); or
138; 263-274; 452-461; 587-595 of AAVrh39 capsid amino acid sequence (SEQ ID NO. 124).

55. A recombinant AAV capsid protein comprising an amino acid sequence TLAAPFK (SEQ ID NO: 2) inserted between amino acid residues 588-589 of the AAV9 capsid (SEQ ID NO:118) or corresponding to between amino acid residues 588 to 589 of the AAV9 capsid as aligned in FIG. 8.

56. A recombinant AAV capsid protein comprising an amino acid sequence TLAAPFK (SEQ ID NO: 2) inserted immediately after one of amino acids I451 to L461 or S268 of the AAV9 capsid (SEQ ID NO:118) or corresponding to one of amino acids I451 to L461 or S268 of the AAV9 capsid as aligned in FIG. 8.

57. A recombinant AAV capsid protein comprising an amino acid sequence QEQLERALNSS (SEQ ID NO: 8) inserted between amino acid residues 588-589 of the AAV9 capsid (SEQ ID NO:118) or corresponding to between amino acid residues 588 to 589 or the AAV9 capsid as aligned in FIG. 8.

58. A recombinant AAV capsid protein comprising an amino acid sequence QEQLERALNSS (SEQ ID NO: 8) inserted immediately after one of amino acids I451 to L461 or S268 of the AAV9 capsid (SEQ ID NO:118) or corresponding to one of amino acids I451 to L461 or S268 of the AAV9 capsid as aligned in FIG. 8.

59. The rAAV capsid protein of any of embodiments 24-54, wherein said peptide insertion occurs immediately after an amino acid residue corresponding to one of amino acids 451 to 461, S268 or Q588 of AAV9 capsid protein (SEQ ID NO:118) as aligned in FIG. 8.

60 The rAAV capsid protein of embodiment 59, wherein said peptide insertion occurs immediately after one of amino acids 451 to 461 of the AAV9 capsid protein (SEQ ID NO:118).

61. The rAAV capsid protein of any of embodiments 24-53, wherein said peptide insertion occurs in the eighth variable region (VR-VIII).

62. The rAAV capsid protein of any of embodiments 24-61, with the proviso that said capsid protein is not the AAV2 capsid protein.

63. A nucleic acid comprising a nucleotide sequence encoding the rAAV capsid protein of any of embodiments 24-62, or encoding an amino acid sequence sharing at least 80% identity therewith.

64. A packaging cell capable of expressing the nucleic acid of embodiment 63 to produce AAV vectors comprising the capsid protein encoded by said nucleotide sequence.

65. A rAAV vector comprising the capsid protein of any of embodiments 24-62.

66. The rAAV vector of embodiment 65 further comprising a transgene.

67. A pharmaceutical composition comprising the rAAV vector of embodiment 65 or 66 and a pharmaceutically acceptable carrier.

68. A method of delivering a transgene to a cell, said method comprising contacting said cell with the rAAV vector of embodiment 66, wherein said transgene is delivered to said cell.

69. A method of delivering a transgene to a target tissue of a subject in need thereof, said method comprising administering to said subject the rAAV vector of embodiment 66;, wherein the transgener is delivered to said subject.

70. A pharmaceutical composition for use in delivering a transgene to a cell, said pharmaceutical composition comprising the rAAV vector of embodiment 66, wherein said transgene is delivered to said cell.

71. A pharmaceutical composition for use in delivering a transgene to a target tissue of a subject in need thereof, said pharmaceutical composition comprising the rAAV vector of embodiment 66; wherein transgene is delivered to the target tissue.

72. The method, or pharmaceutical composition for use, according to embodiments 68-71, wherein said rAAV vector is administered systemically, intravenously, intrathecally, intra-nasally, intra-peritoneally, or intravitreally.

73. The method, or pharmaceutical composition for use, according to embodiments 68-71, wherein said vector is administered via lumbar puncture or via cisterna magna.

74. A recombinant adeno-associated virus (rAAV) capsid protein, said capsid protein comprising a peptide insertion of at least 4 contiguous amino acids from one of TLAVPFK (SEQ ID NO: 27), RTIGPSV (SEQ ID NO: 19), CRTIGPSVC (SEQ ID NO: 20), LGETTRP (SEQ ID NO: 15), or LALGETTRP (SEQ ID NO: 16);

wherein said peptide insertion occurs immediately after an amino acid residue corresponding to amino acids 268, 454 or 588 of AAV9 capsid protein as aligned in FIG. 8.

75. The rAAV capsid protein of embodiment 74, with the proviso that said capsid protein is not the AAV2 capsid protein.

76. The rAAV capsid protein of embodiment 74, wherein said peptide insertion comprises the amino acid sequence TLAVPFK (SEQ ID NO: 27) between amino acid residues 454 and 455 of the AAV9 capsid protein (SEQ ID NO:118).

77. The rAAV capsid protein of embodiment 74, wherein said peptide insertion comprises the amino acid sequence TLAVPFK (SEQ ID NO: 27) immediately after one of amino acid residues 262-273 of the AAV9 capsid protein (SEQ ID NO:118).

78. The rAAV capsid protein of embodiment 74, wherein said peptide insertion comprises the amino acid sequence LGETTRP (SEQ ID NO: 15) between amino acid residues 454-455 of the AAV8 capsid protein (SEQ ID NO:117).

79. The rAAV capsid protein of embodiment 78, wherein said peptide insertion comprises the amino acid sequence LALGETTRP (SEQ ID NO: 16).

80. The rAAV capsid protein of embodiment 74, wherein said peptide insertion comprises the amino acid sequence LGETTRP (SEQ ID NO: 15) inserted between amino acid residues 590-591 of the AAV8 capsid protein (SEQ ID NO:117).

81. The rAAV capsid protein of embodiment 80, wherein said peptide insertion comprises the amino acid sequence LALGETTRP (SEQ ID NO: 16).

82. The rAAV capsid protein of embodiment 74, wherein said peptide insertion comprises the amino acid sequence LGETTRP (SEQ ID NO: 15) immediately after one of amino acid residues 263-274 of the AAV8 capsid protein (SEQ ID NO:117).

83. The rAAV capsid protein of embodiment 82, wherein said peptide insertion comprises the amino acid sequence LALGETTRP (SEQ ID NO: 16).

84. A nucleic acid comprising a nucleotide sequence encoding the rAAV capsid protein of any of embodiments 74-83, or encoding an amino acid sequence sharing at least 80% identity therewith.

85. A packaging cell capable of expressing the nucleic acid of embodiment 84 to produce AAV vectors comprising the capsid protein encoded by said nucleotide sequence.

86. A rAAV vector comprising the capsid protein of any of embodiments 74-83.

87. The rAAV vector of embodiment 86 further comprising a transgene.

88. A pharmaceutical composition comprising the rAAV vector of embodiment 86 or 87 and a pharmaceutically acceptable carrier.

89. A method of delivering a transgene to a cell, said method comprising contacting said cell with the rAAV vector of embodiment 86 or 87, wherein said transgene is delivered to said cell.

90. A method of delivering a transgene to a target tissue of a subject in need thereof, said method comprising administering to said subject the rAAV vector of embodiment 86 or 87, wherein the transgene is delivered to said target tissue.

91. A pharmaceutical composition for use in delivering a transgene to a cell, said pharmaceutical composition comprising the rAAV vector of embodiment 86 or 87, wherein said transgene is delivered to said cell.

92. A pharmaceutical composition for use in delivering a transgene to a target tissue of a subject in need thereof, said pharmaceutical composition comprising the rAAV vector of embodiment 86 or 87, wherein the transgene is delivered to said target tissue.

93. The method, or pharmaceutical composition for use, of embodiments 89-92, wherein said target tissue is retinal cells and the peptide insertion comprises the amino acid sequence LGETTRP (SEQ ID NO: 15) or LALGETTRP (SEQ ID NO: 16).

94. The method, or pharmaceutical composition for use, according to embodiments 89-93, wherein said rAAV vector is administered systemically, intravenously, intrathecally, intra-nasally, intra-peritoneally, or intravitreally.

95. The method, or pharmaceutical composition for use, according to embodiments 89-93, wherein said vector is administered via lumbar puncture or via cisterna magna.

96. A recombinant AAV capsid protein comprising one or more amino acid substitutions relative to the wild type or unengineered capsid protein, in which the rAAV capsid protein is an AAV8 capsid protein (SEQ ID NO:117) with an A269S amino acid substitution or is an AAV9 capsid protein (SEQ ID NO:118) with S263G/S269R/A273T substitutions, or W503R or Q474A substitutions, or corresponding substitutions in a capsid protein of another AAV type capsid.

97. The recombinant AAV capsid protein of embodiment 96 further comprising 498-NNN/AAA-500 for an AAV8 capsid protein or 496-NNN/AAA-498 for an AAV9 capsid protein (SEQ ID NO:118), or corresponding substitutions in a capsid protein of another AAV type capsid.

98. A nucleic acid comprising a nucleotide sequence encoding the rAAV capsid protein of embodiments 96 or 97, or encoding an amino acid sequence sharing at least 80% identity therewith.

99. A packaging cell capable of expressing the nucleic acid of embodiment 98 to produce AAV vectors comprising the capsid protein encoded by said nucleotide sequence.

100. A rAAV vector comprising the capsid protein of any of embodiments 96 or 97.

101. The rAAV vector of embodiment 100 further comprising a transgene.

102. A pharmaceutical composition comprising the rAAV vector of embodiment 100 or 101 and a pharmaceutically acceptable carrier.

103. A method of delivering a transgene to a cell, said method comprising contacting said cell with the rAAV vector of embodiment 101, wherein said transgene is delivered to said cell.

104. A method of delivering a transgene to a target tissue of a subject in need thereof, said method comprising administering to said subject the rAAV vector of embodiment 101, wherein the transgene is delivered to said target tissue.

105. A pharmaceutical composition for use in delivering a transgene to a cell, said pharmaceutical composition comprising the rAAV vector of embodiment 101, wherein said transgene is delivered to said cell.

106. A pharmaceutical composition for use in delivering a transgene to a target tissue of a subject in need thereof, said pharmaceutical composition comprising the rAAV vector of embodiment 101, wherein the transgene is delivered to said target tissue.

107. The method, or pharmaceutical composition for use, according to embodiments 102-106, wherein said rAAV vector is administered systemically, intravenously, intrathecally, intra-nasally, intra-peritoneally, or intravitreally.

108. The method, or pharmaceutical composition for use, according to embodiments 102-106, wherein said vector is administered via lumbar puncture or via cisterna magna.

### 4. BRIEF DESCRIPTION OF THE FIGURES

FIG. 1 depicts sequence comparison of the capsid amino acid sequences including the VR-IV loop of the adeno-associated virus type 9 (AAV9 VR-IV) from residues L447 to R476, (with residues 451-459 bracketed) to corresponding to regions of other AAVs. Figure discloses SEQ ID NOS 87-92, 88, and 93-96, respectively, in order of appearance.

FIG. 2 depicts a protein model of an AAV capsid structure, showing capsid variable regions VR-IV, VR-V and VR-VIII. The box highlights the loop region of VR-IV which provides surface-exposed amino acids as represented in the model.

FIG. 3 depicts high packaging efficiency (titer) in terms of genome copies per mL (GC/mL) of wild type AAV9 and eight (8) candidate modified rAAV9 vectors (1090, 1091, 1092, 1093, 1094, 1095, 1096, and 1097), where the candidate vectors each contain a FLAG insert immediately after different sites within AAV9s VR-IV, from residues I451 to Q458, respectively. All vectors were packaged with luciferase transgene in 10 mL culture; error bars represent standard error of the mean.

FIG. 4 demonstrates surface exposure of 1 VR-IV loop FLAG inserts in each of eight (8) candidate modified rAAV9 vectors (1090, 1091, 1092, 1093, 1094, 1095, 1096, and 1097), confirmed by immunoprecipitation of packaged vectors by binding to anti-FLAG resin.

FIGS. 5A-5B depict transduction efficiency in Lec2 cells, transduced with capsid vectors carrying the luciferase gene (as a transgene), which were packaged into either wild type AAV9 (9-luc), or into each of eight (8) candidate modified (FLAG peptide inserted) rAAV9 vectors (1090, 1091, 1092, 1093, 1094, 1095, 1096, and 1097); transduction activity is expressed as percent luciferase activity, taking the activity of 9-luc as 100% (FIG. 5A), or as Relative Light Units (RLU) per microgram of protein (FIG. 5B).

FIGS. 6A-6E. FIG. 6A depicts a bar graph illustrating that insertions immediately after S454 of AAV9 of varying peptide length and composition may affect production efficiencies of AAV particles in a packaging cell. Ten peptides of varying composition and length were inserted after S454 within AAV9 VR-IV. qPCR was performed on harvested supernatant of transfected suspension HEK293 cells five days post-transfection. The results depicted in the bar graph demonstrate that the nature of the insertions affects the ability of AAV particles to be produced and secreted by HEK293 cells, and indicated by overall yields (titer). (Error bars represent standard error of the mean length of peptide, which is noted on the Y-axis in parenthesis.) FIGS. 6B-6E depict fluorescence images of transduced cell cultures of the following cell lines: (6B) Lec2 cell line (6C) HT-22 cell line, (6D) hCMEC/D3 cell line, and (6E) C2C12 cell line. AAV9 wild type and S454 insertion homing peptide capsids containing GFP transgene were used to transduce the noted cell lines. P1 vector was not included in images due to extremely low transduction efficiency, and P8 vector was not included due to low titer. AAV9.S454.FLAG showed low transduction levels in every cell type tested.

FIGS. 7A-7M depict the amino acid sequences for heavy chain tail domains of human axonemal dyneins 1-12, 14, and 17.

FIG. 8 depicts alignment of AAVs 1-9e, rh10, rh20, rh39, and rh74 version 1 and version 2 capsid sequences with insertion sites for heterologous peptides after the initiation codon of VP2, and within or near variable region 1 (VR-D, variable region 4 (VR-IV), and variable region 8 (VR-VIII), all highlighted in grey; a particular insertion site within variable region eight (VR-VIII) of each capsid protein is shown by the symbol “#” (after amino acid residue 588 according to the amino acid numbering of AAV9).

FIG. 9 depicts the amino acid sequence for a recombinant AAV9 vector including a peptide insertion of ARA290 between Q588 and A589 (SEQ ID NO: 153); the ARA290 insert is shown in bold.

FIG. 10 depicts copies of GFP (green fluorescent protein) transgene in mice brain cells, following administration of the AAV vectors: AAV9; AAV.PHP.eB, also referred to herein as AAV9e (AAV9 with the peptide TLAVPFK (SEQ ID NO: 27) inserted between positions 588 and 589 and modifications A587D/A588G); AAV.hDyn (AAV9 with TLAAPFK (SEQ ID NO: 2) between 588 and 589); AAV.PHP.S (AAV9 with the peptide QAVRTSL (SEQ ID NO: 23) inserted between positions 588 and 589); and AAV.PHP.SH (AAV9 with the peptide QAVRTSH (SEQ ID NO: 24) inserted between positions 588 and 589).

FIGS. 11A-11C depict the amino acid sequences for a recombinant AAV9 vector including a peptide insertion of TLAAPFK (SEQ ID NO: 2) between Q588 and A589 (FIG. 11A), between 5268 and 5269 of VR-III (FIG. 11B), and between S454 and G455 of VR-IV (FIG. 11C), each with the TLAAPFK (SEQ ID NO: 2) insert shown in bold.

FIGS. 12A-12C depict the amino acid sequences for a recombinant AAV9 vector including a peptide insertion of KMQVPFQ (SEQ ID NO: 1) between Q588 and A589 (FIG. 12A), between S268 and S269 of VR-III (FIG. 12B), and between S454 and G455 of VR-IV (FIG. 12C), each with the KMQVPFQ (SEQ ID NO: 1) insert shown in bold.

FIGS. 13A-13C depict the amino acid sequences for a recombinant AAV9 vector including a peptide insertion of QQAAPSF (SEQ ID NO: 3) between Q588 and A589 (FIG. 13A), between S268 and S269 of VR-III (FIG. 13B), and between S454 and G455 of VR-IV (FIG. 13C), each with the QQAAPSF (SEQ ID NO: 3) insert shown in bold.

FIGS. 14A-14C depict the amino acid sequences for a recombinant AAV9 vector including a peptide insertion of RYNAPFK (SEQ ID NO: 4) between Q588 and A589 (FIG. 14A), between S268 and S269 of VR-III (FIG. 14B), and between S454 and G455 of VR-IV (FIG. 14C), each with the RYNAPFK (SEQ ID NO: 4) insert shown in bold.

FIGS. 15A-15C depict the amino acid sequences for a recombinant AAV9 vector including a peptide insertion of LKLPPIV (SEQ ID NO: 5) between Q588 and A589 (FIG. 15A), between S268 and S269 of VR-III (FIG. 15B), and between S454 and G455 of VR-IV (FIG. 15C), each with the LKLPPIV (SEQ ID NO: 5) insert shown in bold.

FIGS. 16A-16C depict the amino acid sequences for a recombinant AAV9 vector including a peptide insertion of PFIKPFE (SEQ ID NO: 6) between Q588 and A589 (FIG. 16A), between S268 and S269 of VR-III (FIG. 16B), and between S454 and G455 of VR-IV (FIG. 16C), each with the PFIKPFE (SEQ ID NO: 6) insert shown in bold.

FIGS. 17A-17C depict the amino acid sequences for a recombinant AAV9 vector including a peptide insertion of TLSLPWK (SEQ ID NO: 7) between Q588 and A589 (FIG. 17A), between S268 and S269 of VR-III (FIG. 17B), and between S454 and G455 of VR-IV (FIG. 17C), each with the TLSLPWK (SEQ ID NO: 7) insert shown in bold.

FIGS. 18A-18C depict the amino acid sequences for a recombinant AAV8 vector including a peptide insertion of LGETTRP (SEQ ID NO: 15) between N590 and T591 (FIG. 18A), between A269 and T270 of VR-III (FIG. 18B), and between T453 and T454 of VR-IV (FIG. 18C), each with the LGETTRP (SEQ ID NO: 15) insert shown in bold.

FIGS. 19A-19C depict the amino acid sequences for a recombinant AAV8 vector including a peptide insertion of LALGETTRP (SEQ ID NO: 16) between N590 and T591 (FIG. 19A), between A269 and T270 of VR-III (FIG. 19B), and between T453 and T454 of VR-IV (FIG. 19C), each with the LALGETTRP (SEQ ID NO: 16) insert shown in bold.

FIGS. 20A-20B depict an in vitro transwell assay for AAV vectors crossing a blood brain barrier (BBB) cell layer (FIG. 20A), and results showing that AAV.hDyn (indicated by inverted triangles) crosses the BBB cell layer of the assay faster than AAV9 (squares), as well as faster and to a greater extent than AAV2 (circles) (FIG. 20B).

FIG. 21 depicts results of Next Generation Sequencing (NGS) analysis of brain gDNA from mice to which pools of engineered and native capsids have been intravenously administered, revealing relative abundances in tissues of the mice of the different capsids in the pool. Three different pools were injected into mice. Dotted lines indicate which vectors were pooled together. Parental AAV9 was included in each pool as control (Pool 1: BC01, Pool 2: BC31, Pool 3: BC01). Bar codes for each capsid of the pool are listed in Table 8a-8c.

FIGS. 22A-22H depict an in vivo transduction profile of AAV.hDyn in female C57B1/6 mice, showing copy number/microgram gDNA in naive mice, or mice injected with either AAV9 or AAV.hDyn in brain (FIG. 22A), liver (FIG. 22B), heart (FIG. 22C), lung (FIG. 22D), kidney (FIG. 22E), skeletal muscle (FIG. 22F), sciatic nerve (FIG. 22G), and ovary (FIG. 22H), where AAV.hDyn shows increased brain bio-distribution compared to AAV9.

FIGS. 23A-23C depict distribution of GFP from AAV.hDyn throughout the brain, where images of immunohistochemical staining of brain sections from the striatum (FIG. 23A), hippocampus (FIG. 23B), and cortex (FIG. 23C) revealed a comprehensive transduction of the brain by the modified vector.

FIG. 24 depicts in vivo kidney to liver transduction efficiency ratio of genetically engineered AAV9 vectors containing insertions of homing peptides immediately after amino acid 454. Details on homing peptides used in this study are outlined in Table 8.

FIG. 25 depicts the amino acid sequences for a recombinant AAV9 vector including a peptide insertion of TLAVPFK (SEQ ID NO: 27) between S454 and G455 of VR-IV with the TLAVPFK (SEQ ID NO: 27) insert shown in bold.

### 5. DETAILED DESCRIPTION

Provided are recombinant adeno-associated viruses (rAAVs) having capsid proteins engineered to include amino acid sequences that confer and/or enhance desired properties, such as tissue targeting, transduction and integration of the rAAV genome. In particular, provided are engineered capsid proteins comprising peptide insertions of 4 to 20, or 7 contiguous amino acids, and in embodiments no more than 12 contiguous amino acids, from heterologous proteins, within or near variable region IV (VR-IV) of the virus capsid, such that the peptide insertion is surface exposed when the capsid protein is packaged as an AAV particle. Also provided are recombinant capsid proteins, and rAAVs comprising them, that have inserted peptides that target specific tissues and/or promote rAAV cellular uptake, transduction and/or genome integration, for example, from the dimerization domain of the heavy chain tail region of human axonemal dynein and others as described herein (see Tables 1A and 1B).

Also provided are engineered capsids having one or more amino acid substitutions that promote transduction and/or tissue tropism described herein. Recombinant vectors comprising the capsid proteins also are provided, along with pharmaceutical compositions thereof, nucleic acids encoding the capsid proteins, and methods of making and using the capsid proteins and rAAV vectors having the engineered capsids for targeted delivery, improved transduction and/or treatment of disorders associated with the target tissue. In particular, provided are compositions comprising rAAVs and methods of using capsid proteins comprising peptides derived from erythropoietin or dynein or that as associated with dynein to target rAAVs to retinal and/or neural tissue, including the central nervous system, and facilitate delivery of therapeutic agents for treating neurological disorders and/or disorders of the eye, particularly, the retina. Also provided are compositions comprising rAAVs comprising peptide insertions that target or home on target tissues, such as bone, kidney, muscle, lung, retina, and heart, as well as methods of using same.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “subject”, “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

As used herein, the terms “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The “central nervous system” (“CNS”) as used herein refers to neural tissue reaches by a circulating agent after crossing a blood-brain barrier, and includes, for example, the brain, optic nerves, cranial nerves, and spinal cord. The CNS also includes the cerebrospinal fluid, which fills the central canal of the spinal cord as well as the ventricles of the brain.

### 5.2. Recombinant AAV Capsids and Vectors

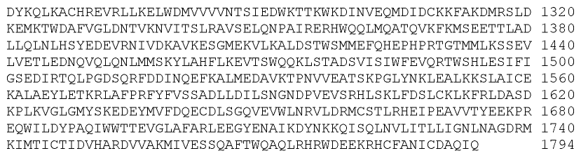

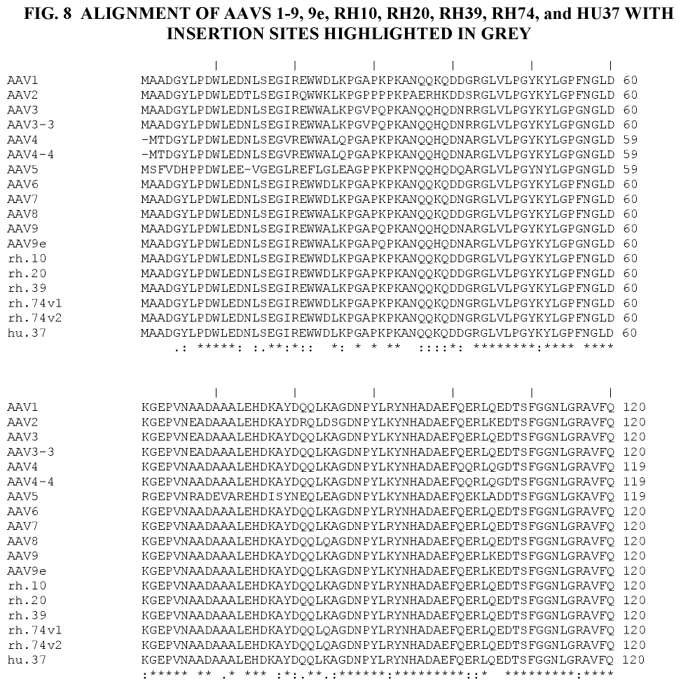

One aspect relates to a capsid protein of a recombinant adeno-associated virus (rAAV), the capsid protein engineered to comprise a peptide insertion from a heterologous protein that is not an AAV protein, where the peptide insertion is surface exposed when packaged as an AAV particle. In some embodiments, the peptide insertion occurs within (i.e., between two amino acids without deleting any capsid amino acids) variable region IV (VR IV) of an AAV9 capsid, or a corresponding region for another type AAV capsid (see alignment in FIG. 8). In some embodiments, the peptide insertion occurs within (i.e., between two amino acids without deleting any capsid amino acids) variable region VIII (VR-VIII) of an AAV9 capsid, or a corresponding region of a capsid for another AAV type (see alignment in FIG. 8). In some embodiments, the peptide insertion is from a heterologous protein or domain (that is not an AAV capsid protein or domain), which directs the rAAV particles to target tissues and/or promote rAAV uptake, transduction and/or genome integration. Also provided are nucleic acids encoding the engineered capsid proteins and variants thereof, packaging cells for expressing the nucleic acids to produce rAAV vectors, rAAV vectors further comprising a transgene, and pharmaceutical compositions of the rAAV vectors, as well as methods of using the rAAV vectors to deliver the transgene to a target cell type or target tissue of a subject in need thereof

In the various embodiments, the target tissue may be neural tissue, bone, kidney, muscle, the eye/retina, or endothelial tissue, or a particular receptor or tumor, and the peptide insertion is derived from a heterologous protein or domain that specifically recognizes and/or binds that tissue, or for example, one or more specific cell types, such as within the target tissue, or cellular matrix thereof. In particular, peptides derived from erythropoietin or dynein, particularly the heavy chain dimerization domain of axonemal dynein or of cytoplasmic dynein, or that bind to or are associated with cytoplasmic dynein inserted into any surface-exposed variable regions, can target rAAVs to neural tissue, including crossing the blood brain barrier to the CNS and delivering therapeutics for treating neurological disorders.

### 5.2.1 rAAV Vectors with Peptide Insertions

The present inventors have surprisingly discovered positions amenable to peptide insertions within and near the AAV9 capsid VR-IV loop (see FIG. 2) and corresponding regions on the VR-IV loop of capsids of other AAV types. Though previous studies analyzed potential positions in various AAVs, none identified the AAV9 VR-IV as amenable for this purpose (consider, e.g., Wu et al, 2000, “Mutational Analysis of the Adeno-Associated Virus Type 2 (AAV2) Capsid Gene and Construction of AAV2 Vectors with Altered Tropism,” J of Virology 74(18):8635-8647 ; Lochrie et al, 2006, “Adeno-associated virus (AAV) capsid genes isolated from rat and mouse liver genomic DNA define two new AAV species distantly related to AAV-5,” Virology 353:68-82; Shi and Bartlett, 2003, “RGD Inclusion in VP3 Provides Adeno-Associated Virus Type 2 (AAV2)-Based Vectors with a Heparan Sulfate-Independent Cell Entry Mechanism,” Molecular Therapy 7(4):515525-; Nicklin et al., 2001, “Efficient and Selective AAV2-Mediated Gene Transfer Directed to Human Vascular Endothelial Cells” Molecular Therapy 4(2):174-181; Grifman et al., 2001, “Incorporation of Tumor-Targeting Peptides into Recombinant Adeno-associated Virus Capsids,” Molecular Therapy 3(6):964-975; Girod et al. 1999, “Genetic capsid modifications allow efficient re-targeting of adeno-associated virus type 2,” Nature Medicine 3(9):1052-1056; Douar et al., 2003, “Deleterious effect of peptide insertions in a permissive site of the AAV2 capsid, “Virology 309:203-208; and Ponnazhagan, et al. 2001, J. of Virology 75(19):9493-9501).

Accordingly, provided are rAAV vectors carrying peptide insertions at novel insertion points, in particular, within surface-exposed variable regions in the capsid coat, particularly within or near the variable region IV of the capsid protein. In some embodiments, the rAAV capsid protein comprises a peptide insertion immediately after (i.e., connected by a peptide bond C-terminal to) an amino acid residue corresponding to one of amino acids 451 to 461 of AAV9 capsid protein (amino acid sequence SEQ ID NO:118 and see FIG. 8 for alignment of capsid protein amino acid sequence of other AAV serotypes with amino acid sequence of the AAV9 capsid), where said peptide insertion is surface exposed when the capsid protein is packaged as an AAV particle. The peptide insertion should not delete any residues of the AAV capsid protein. Generally, the peptide insertion occurs in a variable (poorly conserved) region of the capsid protein, compared with other serotypes, and in a surface exposed loop.

A peptide insertion described as inserted “at” a given site refers to insertion immediately after, that is having a peptide bond to the carboxy group of, the residue normally found at that site in the wild type virus. For example, insertion at Q588 in AAV9 means that the peptide insertion appears between Q588 and the consecutive amino acid (A589) in the AAV9 wildtype capsid protein sequence (SEQ ID NO:118). In embodiments, there is no deletion of amino acid residues at or near (within 5, 10, 15 residues or within the structural loop that is the site of the insertion) the point of insertion.

In particular embodiments, the capsid protein is an AAV9 capsid protein and the insertion occurs immediately after at least one of the amino acid residues 451 to 461. In particular embodiments, the peptide insertion occurs immediately after amino acid I451, N452, G453, S454, G455, Q456, N457, Q458, Q459, T460, or L461 of the AAV9 capsid (amino acid sequence SEQ ID NO: 118). In certain embodiments, the peptide is inserted between residues S454 and G455 of AAV9 capsid protein or between the residues corresponding to S454 and G455 of an AAV capsid protein other than an AAV9 capsid protein (amino acid sequence SEQ ID NO: 118).

In other embodiments, provided are engineered capsid proteins comprising targeting peptides heterologous to the capsid protein that are inserted into the AAV capsid protein such that, when incorporated into the AAV vector the heterologous peptide is surface exposed. Such peptides are preferably from human axonemal dynein (HAD) heavy chain tail or are those listed in Tables 1A and 1B below or other targeting peptides for specific tissue types.

In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), and serotype rh74 (AAVrh74, versions 1 and 2) (see FIG. 8), and the insertion occurs immediately after an amino acid residue corresponding to at least one of the amino acid residues 451 to 461. The alignments of these different AAV serotypes, as shown in FIG. 8, indicates “corresponding” amino acid residues in the different capsid amino acid sequences such that a “corresponding” amino acid residue is lined up at the same position in the alignment as the residue in the reference sequence. In some particular embodiments, the peptide insertion occurs immediately after one of the amino acid residues within: 450-459 of AAV1 capsid (SEQ ID NO: 110); 449-458 of AAV2 capsid (SEQ ID NO: 111); 449-459 of AAV3 capsid (SEQ ID NO: 112); 443-453 of AAV4 capsid (SEQ ID NO: 113); 442-445 of AAV5 capsid (SEQ ID NO: 114); 450-459 of AAV6 capsid (SEQ ID NO: 115); 451-461 of AAV7 capsid (SEQ ID NO: 116); 451-461 of AAV8 capsid (SEQ ID NO: 117); 451-461 of AAV9 capsid (SEQ ID NO: 118); 452-461 of AAV9e capsid (SEQ ID NO: 119); 452-461 of AAVrh10 capsid (SEQ ID NO: 120); 452-461 of AAVrh20 capsid (SEQ ID NO: 121); 452-461 of AAVhu.37 (SEQ ID NO: 122); 452-461 of AAVrh74 (SEQ ID NO: 123 or SEQ ID NO: 154); or 452-461 of AAVrh39 (SEQ ID NO: 124), in the sequences depicted in FIG. 8. In certain embodiments, the rAAV capsid protein comprises a peptide insertion immediately after (i.e., C-terminal to) amino acid 588 of AAV9 capsid protein (having the amino acid sequence of SEQ ID NO:118 and see FIG. 8), where said peptide insertion is surface exposed when the capsid protein is packaged as an AAV particle. In other embodiments, the rAAV capsid protein has a peptide insertion that is not immediately after amino acid 588 of AAV9 or corresponding to amino acid 588 of AAV9.

In other embodiments, when the peptide is a targeting peptide, including, at least 4 contiguous amino acids, or at least 7 contiguous amino acids, or is exactly 7 contiguous amino acids, but, in embodiments, no more than 12 contiguous amino acids, or functional fragments thereof, of Tables 1A and 1B, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), and serotype rh74 (AAVrh74, versions 1 and 2) (see FIG. 8), and the peptide is inserted in the capsid protein at any point such that the peptide is surface exposed when incorporated into the AAV vector. In specific embodiments, the peptide is inserted after 138; 262-272; 450-459; or 585-593 of AAV1 capsid (SEQ ID NO: 110); 138; 262-272; 449-458; or 584-592 of AAV2 capsid (SEQ ID NO: 111); 138; 262-272; 449-459; or 585-593 of AAV3 capsid (SEQ ID NO: 112); 137; 256-262; 443-453; or 583-591 of AAV4 capsid (SEQ ID NO: 113); 137; 252-262; 442-445; or 574-582 of AAV5 capsid (SEQ ID NO: 114); 138; 262-272; 450-459; 585-593 of AAV6 capsid (SEQ ID NO: 115); 138; 263-273; 451-461; 586-594 of AAV7 capsid (SEQ ID NO: 116); 138; 263-274; 452-461; 587-595 of AAV8 capsid (SEQ ID NO: 117); 138; 262-273; 452-461; 585-593 of AAV9 capsid (SEQ ID NO: 118); 138; 262-273; 452-461; 585-593 of AAV9e capsid (SEQ ID NO: 119); 138; 263-274; 452-461; 587-595 of AAVrh10 capsid (SEQ ID NO: 120); 138; 263-274; 452-461; 587-595 of AAVrh20 capsid (SEQ ID NO: 121); 138; 263-274; 452-461; 587-595 of AAVrh74 capsid (SEQ ID NO: 123 or SEQ ID NO: 154), 138; 263-274; 452-461; 587-595 of AAVhu37 capsid (SEQ ID NO: 122); or 138; 263-274; 452-461; 587-595 of AAVrh39 capsid (SEQ ID NO: 124) (as numbered in FIG. 8).

In some embodiments, the capsid protein is from an AAV other than serotype AAV2. In some embodiments, the peptide insertion does not occur immediately after an amino acid residue corresponding to amino acid 570 or 611 of AAV2 capsid protein. In some embodiments, the peptide insertion does not occur between amino acid residues corresponding to amino acids 587-588 of AAV2 capsid protein (see US 2014/0294771 to Schaffer et al). In some embodiments, the insertion of the bonel peptide with amino acid sequence DDDDDDDD (SEQ ID NO: 9) does not occur directly after amino acid 138 of AAV2 capsid protein (see Alméciga-Diaz et al., 2018, Pediatr. Res.84:545).

Also provided are AAV vectors comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereofserotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. These engineered AAV vectors may comprise a genome comprising a transgene encoding a therapeutic protein.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat Nos. 8,628,966; 8,927,514; 9,923,120 and WO 2016/049230, such as HSC1, HSC2, HSC3, HSC4, HSCS, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18(2):171-82; McCarty et al, 2001, Gene Therapy, 8(16):1248-1254; U.S. Pat. Nos. 6,596,535; 7,125,717; and 7,456,683, each of which is incorporated herein by reference in its entirety).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to any AAV serotype capsid. Non-limiting examples include a peptide of a human protein in an AAV capsid protein.

In some embodiments, the peptide insertion is from a homing protein or a homing domain thereof or targeting protein or targeting domain thereof. A “homing domain” or “homing protein” is a domain or protein that preferentially or selectively targets a particular cell type, including cell matrix of a particular cell type—tissue type, organ, tumor type, or the like, over other cells, tissues, organs, or tumors. In the context of the present invention, a peptide from a homing protein or domain gives a peptide for being inserted into a capsid protein, to form part of a capsid coat, or AAV vector, which can then direct the capsid, coat, or vector to target the particular cell type, tissue type, organ, tumor type, or the like or to promote uptake and/or integration of the AAV genome. Non-limiting examples of homing proteins or domains include neural tissue-homing domains, axonemal or cytoplasmic dynein-homing domains, bone-homing domains, kidney-homing domains, muscle-homing domains, endothelial cell-homing domains, retinal cell-homing domains, domains that target particular cellular receptors, such as integrin receptor-binding domains and transferrin receptor-binding domains, tumor cell-targeting domains, targeting peptides from other viruses and the like. As used herein, the terms “homing” and “targeting” are used interchangeably. These peptides may also or alternatively promote rAAV cell uptake, transduction and/or genome integration in cells of the target tissue.

Examples of peptides for use as peptide insertions as any of the AAV capsid sites described herein are presented in Tables 1A-1B below and include at least 4 amino acid contiguous portions thereof, or 7 amino acid contiguous portions thereof and in some embodiments no more than 12 contiguous amino acids that have the functional attribute of the peptide. See also, e.g., Laakkonen and Vuorinen, 2010, “Homing peptides as targeted delivery vehicles,” Integrative Biology, 2:326-337 (review article). In certain embodiments, the recombinant AAV capsids and AAV vectors are engineered to include a peptide, or at least 4, 5, 6, or 7 amino acid contiguous portion thereof, from any of Tables 1A and 1B below, inserted into the AAV capsid sequence in such a way that the peptide insertion is displayed. In other embodiments, the peptides are inserted after an amino acid residue at positions 138, 262-273, 451-461, or 585-593 of the amino acid sequence of the AAV9 capsid (SEQ ID NO: 118) or a position corresponding thereto in any other AAV serotype (see FIG. 8 for capsid sequence alignments).

TABLE 1A
Homing Peptides

| Peptide | SEQ | | | | |
|---|---|---|---|---|---|
| Sequence | ID | | Target | | |
| (# of aa) | NO: | Name | Tissue | Target cell | Receptor |
| CLSSRLDAC (9) | 11 | SRL | Brain | NR | NR |
| CLPVASC (7) | 12 | | Kidney | NR | NR |
| CGFERVRQCPERC | 31 | GFE-1 | Lung | Alveolar | Membrane |
| (13) | | | | capillaries | dipeptidase |
| CGFELETC (8) | 32 | GFE-2 | | | (MDP) |
| CVALCREACGEGC | 33 | | Skin | Hypodermal blood | NR |
| (13) | | | | vasculature | |
| SWCEPGWCR (9) | 34 | | Pancreas, | Capillaries and | NR |
| | | | homes | larger vessels of | |
| | | | also to | uterus, exocrine | |
| | | | uterus | pancreas and islets | |
| YSGKWGW (7) | 35 | | Intestine | NR | NR |
| GSLGGRS (7) | 36 | | Uterus | NR | NR |
| LMLPRAD (7) | 37 | | Adrenal | NR | NR |
| | | | gland | | |
| CKCCRAKDC (9) | 38 | | White fat | Blood vasculature | Prohibitin |
| ASSLNIA (7) | 14 | | Muscle | Muscle fibers | NR |
| SMSIARL (7) | 39 | SMS | Prostate | NR | NR |
| CRPPR (5) | 40 | | Heart | Blood vasculature | CRIP2; HLP; |
| | | | | | ESP-1 |
| CKRAVR (5) | 41 | | Heart | Blood vasculature | Sigirr; TIRS |
| CPKTRRVPC (9) | 42 | | Heart | Blood vasculature | bc10 |
| CRSTRANPC (9) | 43 | | Heart | Blood vasculature | MpcII-3 |
| CARPAR (6) | 44 | | Heart | Blood vasculature | EST |
| CPGPEGAGC (9) | 45 | | Breast | Blood vasculature | Aminopeptidase |
| | | | | | P |

TABLE 1B

| | SEQ ID | | |
|---|---|---|---|
| Peptide Sequence | NO: | Name | Target |
| DDDDDDDD | 9 | Bone1 | Bone |
| LSSRLDA | 10 | Brain1 | Brain |
| CLSSRLDAC | 11 | Brain2 | Brain |
| SITLVKSTQTV | 21 | DLC-AS1 | Dynein light chain |
| TILSRSTQTG | 22 | DLC-AS2 | Dynein light chain |
| VVMVGEKPITITQHSVETEG | 25 | DLC-AS3 | Dynein light chain |
| RSSEEDKSTQTT | 26 | DLC-AS4 | Dynein light chain |
| KSTEDKSTQTP | 46 | | Dynein light chain |
| LGHFTRSTQTS | 47 | | Dynein light chain |
| GVQMAKSTQTF | 48 | | Dynein light chain |
| PKTRNSQTQTD | 49 | | Dynein light chain |
| VTTQNTASQTM | 50 | | Dynein light chain |
| KSSQDKSTQTTGD | 51 | | Dynein light chain |
| KMQVPFQ | 1 | DYH1 | Dynein peptide; neuronal, |
| | | | brain, and retinal cells |
| TLAAPFK | 2 | hDyn or | Dynein peptide; neuronal, |
| | | DYH3 | brain, and retinal cells |
| LKLPPIV | 5 | DYH12.1 | Dynein peptide; neuronal, |
| | | | brain, and retinal cells |
| PFIKPFE | 6 | DYH12.2 | Dynein peptide; neuronal, |
| | | | brain, and retinal cells |
| TLSLPWK | 7 | DYH17 | Dynein peptide; neuronal, |
| | | | brain, and retinal cells |
| QQAAPSF | 3 | DYH7 | Dynein peptide; neuronal, |
| | | | brain, and retinal cells |
| RYNAPFK | 4 | DYH8 | Dynein peptide; neuronal, |
| | | | brain, and retinal cells |
| QEQLERALNSS | 8 | EPO-HBSP | ARA290 |
| DYKDDDDK | 52 | FLAG | None (FLAG) |
| LPVAS | 13 | Kidney1 | Kidney |
| CLPVASC | 12 | Kidney2 | Kidney |
| ASSLNIA | 14 | Muscle1 | Muscle |
| TLAVPFK | 27 | PHP.B | Ly-6a binding domain |
| QAVRTSL | 23 | PHP.S | |
| QAVRTSH | 24 | PHP.SH | |
| HAIYPRH | 17 | TfR1 | Transferrin Receptor |
| THRPPMWSPVWP | 18 | TfR2 | Transferrin Receptor |
| RTIGPSV | 19 | TfR3 | Transferrin Receptor |
| CRTIGPSVC | 20 | TfR4 | Transferrin Receptor |
| LGETTRP | 15 | Retinal Cell1 | Retinal cells |
| LALGETTRP | 16 | Retinal Ce112 | Retinal cells |

In another aspect, provided are heterologous peptide insertion libraries. A heterologous peptide insertion library refers to a collection of rAAV vectors that carry the same peptide insertion at different insertion sites in the virus capsid, e.g., at different positions within a given variable region of the capsid. Generally, the capsid proteins used comprise AAV genomes that contain modified rep and cap sequences to prevent the replication of the virus under conditions in which it could normally replicate (co-infection of a mammalian cell along with a helper virus such as adenovirus). The members of the peptide insertion libraries may then be assayed for functional display of the peptide on the rAAV surface, tissue targeting and/or gene transduction.

The present inventors also have surprisingly discovered particular peptides that can be used to re-target AAV vectors to specific tissues, organs, or cells; in particular, providing peptides that cause rAAV vectors to target retinal tissue and/or to cross the blood-brain barrier and target neural tissue of the CNS. Without being bound by any one theory, certain peptides inserted in an AAV capsid variable region loop, such as dynein and transferrin-derived peptides were demonstrated to enhance transduction efficiency in the brain or retina and/or enhance transport of AAV particles carrying transgene across an endothelial cellular matrix, in particular across laminin-rich basement membranes such as the blood-brain barrier and the inner limiting membrane of the retina. This can provide enhanced transport of AAV particles encapsidating a transgene across an endothelial cellular matrix. Such peptides, and others, are described below.

### 5.2.2 Neural tissue-homing Peptides

Another aspect of the present invention relates to capsid proteins comprising peptide insertions designed to confer or enhance neurotropic properties. Neural tissue includes, but is not limited to, neurons, astrocytes, glia, endothelial cells, and the laminin-rich basement cellular matrix protecting the brain. The invention involves engineering rAAV capsids to display peptides that promote targeting neuronal tissue and neuronal transduction. Examples include peptides from (i) a region of human axonemal dynein (HAD) heavy chain tail; or a region of mouse axonemal dynein (MAD) heavy chain tail; (ii) an erythropoietin (EPO) domain that binds innate repair receptor and is not erythropoietic, or a conformational analog of said domain; and (iii) brain targeting peptides.

### 5.2.2.1 rAAV-HAD Vectors

In certain embodiments, the peptide insertion is a peptide derived from regions of human axonemal dynein (HAD) heavy chain tail and the insertion is used as a neural tissue-homing peptide (or neural cell-homing peptide) and/or a retinal cell-homing peptide (aspects of which are discussed in more detail below). The peptide, referred to herein as a “HAD peptide” may be a sequence of at least 4 consecutive amino acids from HAD heavy chain tail region, or a conformation analog designed to mimic the three-dimensional structure thereof. Recombinant AAV vectors comprising one or more HAD peptides, e.g., inserted into a surface-exposed loop of an AAV capsid coat, are referred to herein as “rAAV-HAD vectors.”

Dyneins are cytoskeletal motor proteins that move along microtubules. There are two basic types: (1) Cytoplasmic dyneins and (2) Axonemal dyneins. Cytoplasmic dyneins function in transporting intracellular cargos and movement of chromosomes on mitotic spindles. Cytoplasmic dyneins usually occur as dimers of two identical heavy chains and several intermediate and light chains. Axonemal dyneins cause sliding of microtubules in axonemes, including structures in cilia and flagella. Axonemal dyneins are found in multiple forms, containing one, two or three non-identical heavy chains.

The overall structure of human axonemal dynein (HAD) involves “tail” and “head” regions. The tail comprises a dimerization domain, which recruits cargos for transport along microtubules. The head comprises a motor domain, which is composed of six AAA domains (triple ATPases) that are “force-generating” and drive the dynein motor to attach and detach, and thus “walk” along, the surface of microtubules. See, also, Toda et al., 2018, Biophysical Rev 10:677-686; Reck-Peterson, 2018, Nat Rev Mol Cell Biol; Reck-Peterson et al., 2006, “Single-Molecule Analysis of Dynein Processivity and Stepping Behavior,” Cell 126:335-348; Urnavicius, 2018, Nature 554:202; Urnavicius, 2015, Science 347:1441; and Zhang et al, 2017, Cell 169:1303. Additionally, see, e.g., Roberts, et al., 2013, “Functions and mechanics of dynein motor proteins” Nat Rev Mol Cell Biol., 14(11):713-726; Wadsworth et al., 2013, “Microtubule Motors: Doin; It without Dynactin,” Curr Biol 23(13):R563-R565; Kelkar et al., 2006, “A Common Mechanism for Cytoplasmic Dynein-Dependent Microtubule Binding Shared among Adeno-Associated Virus and Adenovirus Serotypes,”J. of Virology, 7781-7785; and Zhang et al., 2017, “Cryo-EM Reveals How Human Cytoplasmic Dynein Is Auto-Inhibited and Activated,” Cell, 169:1303-1314.

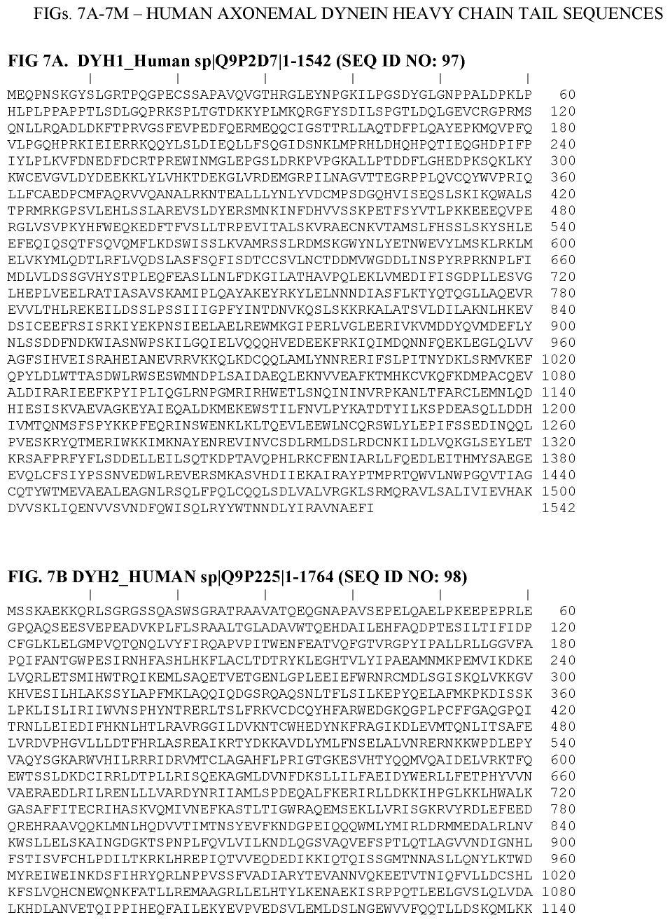

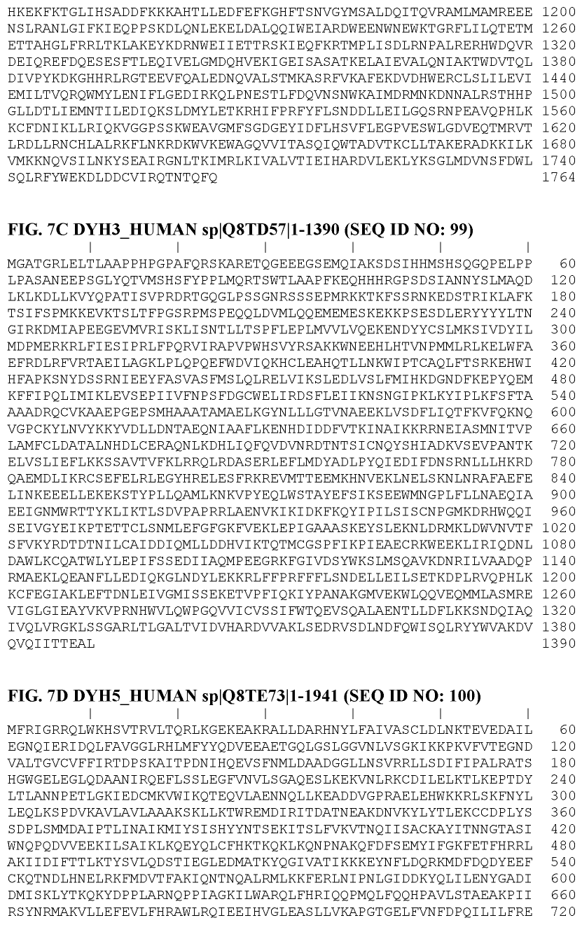

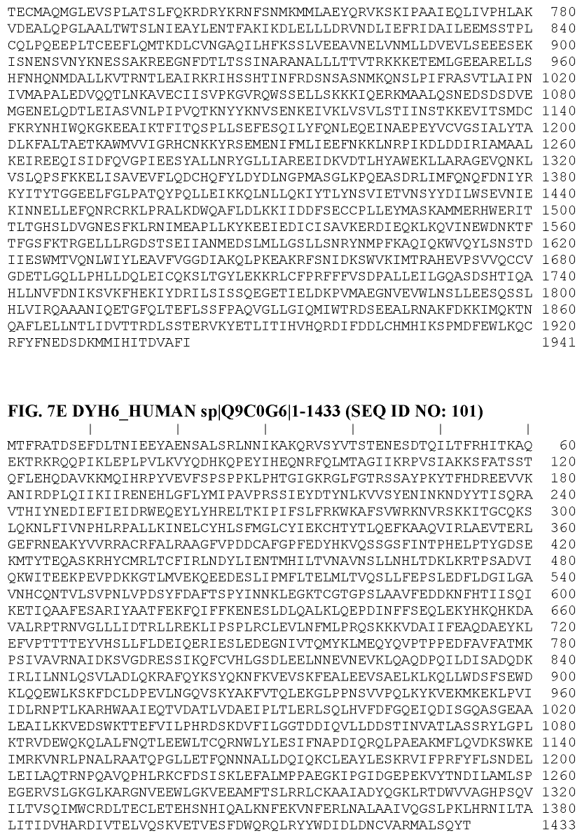

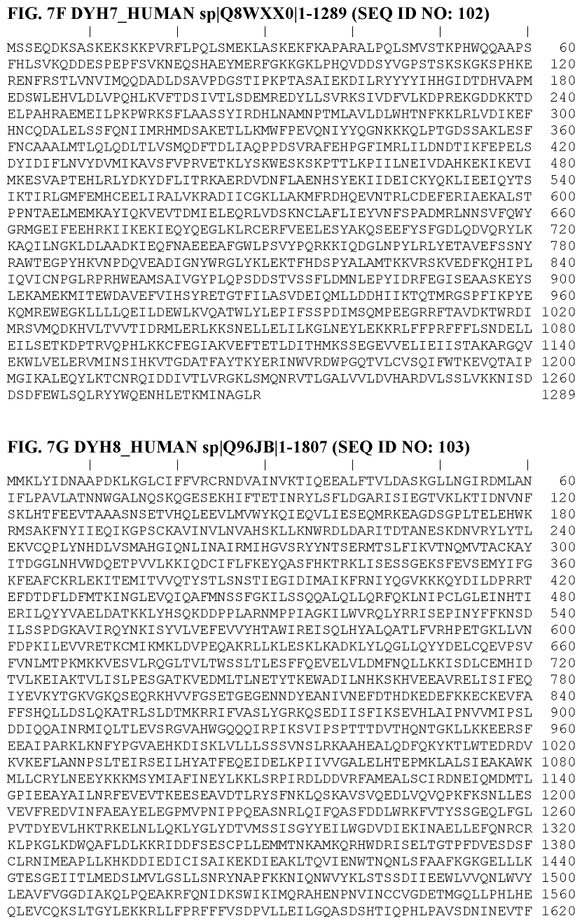

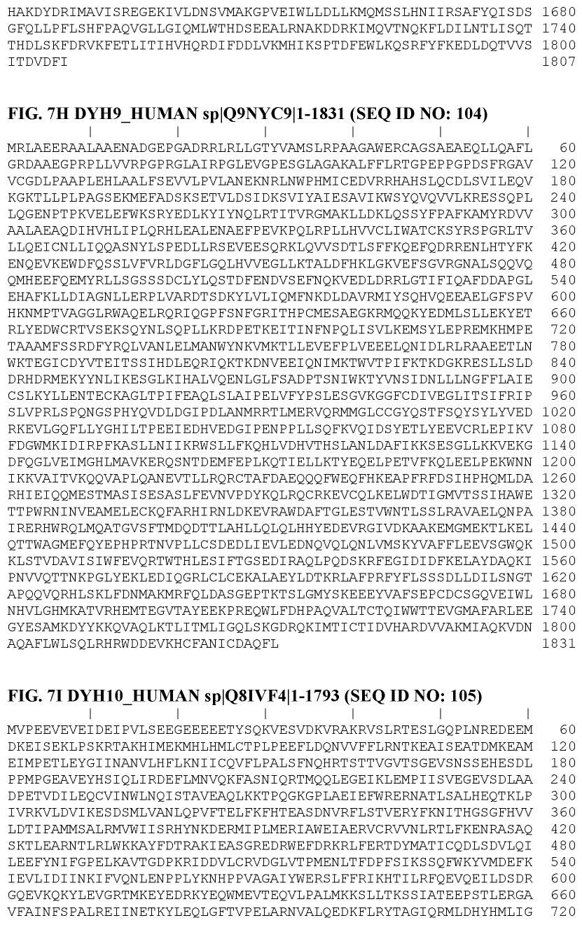

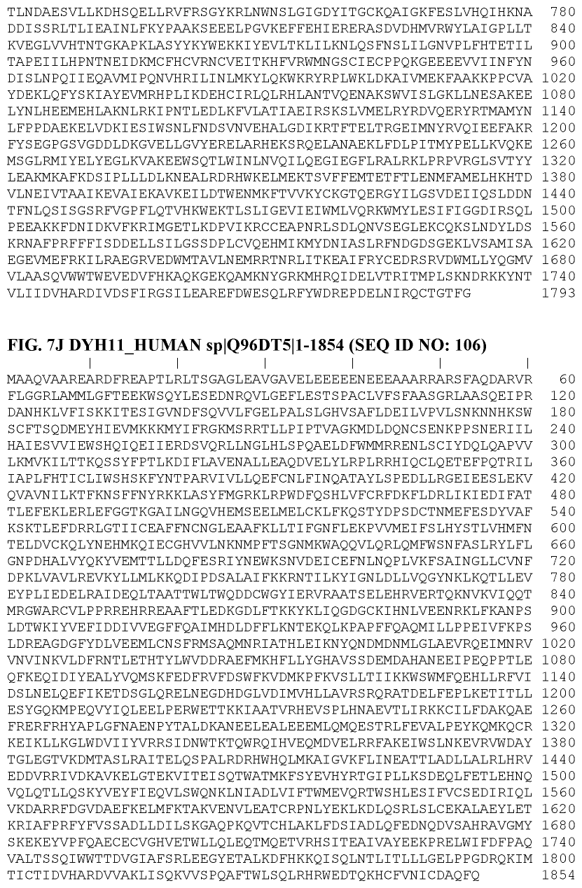

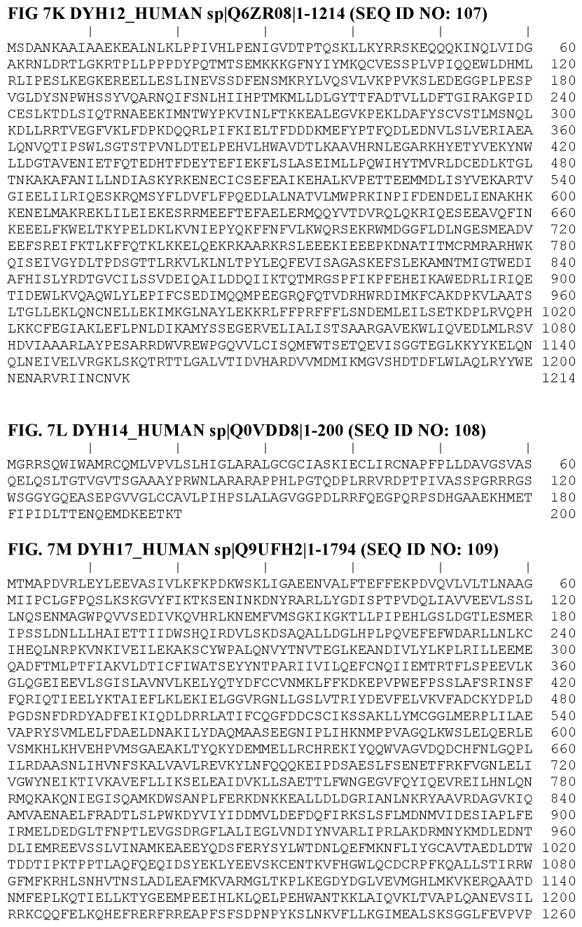

Table 2 identifies the tail and dimerization domain of the human axonemal dyneins, as well as peptides for use as peptide insertions in the engineered capsid proteins described herein. In some embodiments, insertions of at least 4 and up to 15 contiguous amino acids, or 7 contiguous amino acids, from the axonemal dynein sequences of the stem/tail region and/or the dimerization domain (NDD) are used (see also FIGS. 7A-7M).

TABLE 2
Axonemal Dynein Peptides

| | | | | SEQ |
|---|---|---|---|---|
| | STEM/TAIL | NDD | | ID |
| UniProt | SEQUENCE | SEQUENCE | Peptides | NO: |
| DYH1_HUMAN (Q9P2D7) | 1-1542 | 1-200 | K¹⁷⁴MQVPFQ | 1 |
| (SEQ ID NO. 97) | | | | |
| DYH2_HUMAN (Q9P225) | 1-1764 | 1-200 | | |
| (SEQ ID NO. 98) | | | | |
| DYH3_HUMAN (Q8TD57) | 1-1390 | 1-200 | T⁹²LAAPFK | 2 |
| (SEQ ID NO. 99) | | | | |
| DYH5_HUMAN (Q8TE73) | 1-1941 | 1-200 | | |
| (SEQ ID NO. 100) | | | | |
| DYH6_HUMAN (Q9C0G6) | 1-1433 | 1-200 | | |
| (SEQ ID NO. 101) | | | | |
| DYH7_HUMAN (Q8WXX0) | 1-1289 | 1-200 | Q⁵⁵QAAPSF | 3 |
| (SEQ ID NO. 102) | | | | |
| DYH8_HUMAN (Q96JB1) | 1-1807 | 1-200 | R¹⁴⁶⁵YNAPFK | 4 |
| (SEQ ID NO. 103) | | | | |
| DYH9_HUMAN (Q9NYC9) | 1-1831 | 1-200 | | |
| (SEQ ID NO. 104) | | | | |
| DYH10_HUMAN (Q8IVF4) | 1-1793 | 1-200 | | |
| (SEQ ID NO. 105) | | | | |
| DYH11_HUMAN (Q96DT5) | 1-1854 | 1-200 | | |
| (SEQ ID NO. 106) | | | | |
| DYH12_HUMAN (Q6ZR08) | 1-1214 | 1-200 | L¹⁸KLPPIV | 5 |
| (SEQ ID NO. 107) | | | P⁸⁷⁹FIKPFE | 6 |
| DYH14_HUMAN | | 1-200 | | |
| (Q0VDD8) (SEQ ID NO. | | | | |
| 108) | | | | |
| DYH17_HUMAN (Q9UFH2) | 1-1794 | 1-200 | T⁸⁵⁴LSLPWK | 7 |
| (SEQ ID NO. 109) | | | | |

In some embodiments, the peptide for insertion in an AAV capsid is designed from the dimerization domain (NDD) of a HAD heavy chain tail region. In alternate embodiments, peptides corresponding to the amino acid sequences of the remainder of the HAD heavy chain tail (i.e., excluding the dynein motor domain) can be used. In some embodiments, the peptide insertion comprises at least 4, in an embodiment, is 7, contiguous amino acids, and is up to 12 or 15 contiguous amino acids from a dimerization domain of a HAD heavy chain tail. In particular embodiments, the peptide insertion comprises at least 4, is 7 contiguous amino acids, and is up to 12 or 15 contiguous amino acids from the group consisting of (depicted in FIGS. 7A-7M): amino acids (“aa”) 1-1542 of DYH1_HUMAN UniProtKB-Q9P2D7 (SEQ ID NO. 97); aa 1-1764 of DYH2_HUMAN UniProtKB-Q9P225 (SEQ ID NO. 98); aa 1-1390 of DYH3_HUMAN UniProtKB-Q8TD57 (SEQ ID NO. 99); aa 1-1941 of DYH5_HUMAN UniProtKB-Q8TE73 (SEQ ID NO. 100); aa 1-1433 of DYH6_HUMAN UniProtKB-Q9C0G6 (SEQ ID NO. 101); aa 1-1289 of DYH7_HUMAN UniProtKB-Q8WXX0 (SEQ ID NO. 102); aa 1-1807 of DYH8_HUMAN UniProtKB-Q96JB1 (SEQ ID NO. 3); aa 1-1831 of DYH9_HUMAN UniProtKB-Q9NYC9 (SEQ ID NO. 104); aa 1-1793 of DYH10_HUMAN UniProtKB-Q8IVF4 (SEQ ID NO. 105); aa 1-1854 of DYH11_HUMAN UniProtKB-Q96DT5 (SEQ ID NO. 106); aa 1-1214 of DYH12_HUMAN UniProtKB-Q6ZR08 (SEQ ID NO. 107); aa 1-200 of DYH14_HUMAN UniProtKB-Q0VDD8 (SEQ ID NO. 108); and aa 1-1794 of DYH17_HUMAN UniProtKB-Q9UFH2 (SEQ ID NO. 109)) and promotes neural tissue tropism and/or transduction of the capsid engineered to contain the peptide. In more preferred embodiments, the peptide insertion comprises at least 4 contiguous amino acids, is 7 contiguous amino acids, and is up to 12 or 15 contiguous amino acids from residues 1-200 of any one of the dynein heavy chain sequences recited above, that is, any one from the group consisting of aa 1-1542 of DYH1_HUMAN UniProtKB-Q9P2D7 (SEQ ID NO. 97); aa 1-1764 of DYH2_HUMAN UniProtKB-Q9P225 (SEQ ID NO. 98); aa 1-1390 of DYH3_HUMAN UniProtKB-Q8TD57 (SEQ ID NO. 99); aa 1-1941 of DYH5_HUMAN UniProtKB-Q8TE73 (SEQ ID NO. 100); aa 1-1433 of DYH6_HUMAN UniProtKB-Q9C0G6 (SEQ ID NO. 101); aa 1-1289 of DYH7_HUMAN UniProtKB-Q8WXX0 (SEQ ID NO. 102);; aa 1-1807 of DYH8_HUMAN UniProtKB-Q96JB1 (SEQ ID NO. 3); aa 1-1831 of DYH9_HUMAN UniProtKB-Q9NYC9 (SEQ ID NO. 104); aa 1-1793 of DYH10_HUMAN UniProtKB-Q8IVF4 (SEQ ID NO. 105); aa 1-1854 of DYH11_HUMAN UniProtKB-Q96DT5 (SEQ ID NO. 106); aa 1-1214 of DYH12_HUMAN UniProtKB-Q6ZR08 (SEQ ID NO. 107); aa 1-200 of DYH14_HUMAN UniProtKB-Q0VDD8 (SEQ ID NO. 108); and aa 1-1794 of DYH17_HUMAN UniProtKB-Q9UFH2 (SEQ ID NO. 109))) and promotes neural tissue or specific neural cell tropism and/or transduction of the capsid engineered to contain the peptide. In still more preferred embodiments, the peptide insertion is 7 contiguous amino acids from any one of the dynein heavy chain sequences of FIGS. 7A-7M, or is 7 contiguous amino acids from residues 1-200 of any one of the dynein heavy chain sequences (FIGS. 7A-7M).

In particular embodiments, the peptide insertion is at least or consists of 4, 5, 6, or 7 contiguous amino acids from the group consisting of: KMQVPFQ (SEQ ID NO: 1); TLAAPFK (SEQ ID NO: 2); QQAAPSF (SEQ ID NO: 3); RYNAPFK (SEQ ID NO: 4); LKLPPIV (SEQ ID NO: 5); PFIKPFE (SEQ ID NO: 6); and TLSLPWK (SEQ ID NO: 7) and promotes neural tissue tropism and/or transduction of the capsid engineered to contain the peptide. In still more particular embodiments, the peptide insertion consists of a peptide from the group consisting of: KMQVPFQ (SEQ ID NO: 1); TLAAPFK (SEQ ID NO: 2); QQAAPSF (SEQ ID NO: 3); RYNAPFK (SEQ ID NO: 4); LKLPPIV (SEQ ID NO: 5); PFIKPFE (SEQ ID NO: 6); and TLSLPWK (SEQ ID NO: 7) and promotes neural tissue tropism and/or transduction of the capsid engineered to contain the peptide. In one embodiment of particular interest, the peptide insertion comprises or consists of the amino acid sequence TLAAPFK (SEQ ID NO: 2).

While not wishing to be bound to any theory, the rAAV-HAD vectors of the present invention are based on the principle that the rAAV capsid with the incorporated peptide will display multiple copies of the human dynein dimerization domain on the rAAV surface. Upon transduction of a target human cell, such rAAVs may be loaded onto endogenous axonemal dynein in the target cell directly or via recruitment by dynein adaptors in the cell. Loading of such rAAVs onto axonemal dynein may facilitate dynein multimerization and/or stabilize conformation of the dynein to enhance transport activity.

The selection of peptide domains from human axonemal heavy chain dynein for incorporation into AAV capsids to promote rAAV binding to the dynein itself, while counter-intuitive, provides several advantages:
(1) Axonemal dyneins occur in ciliary neurons and, therefore, the rAAV-HAD vectors of the invention may demonstrate enhanced neurotropic properties in sensory neurons, olfactory neurons, auditory neurons, and photoreceptors which contain such structures. Targeting axonemal dynein, as opposed to cytoplasmic dynein which occurs in all cells, may also confer increased selectivity for neural tissue;
(2) Surprisingly, the heavy chain dimerization peptide does not interfere with activity of the axonemal dynein motor domain (in contrast to prior failed attempts to engineer AAV2 capsid using synthetic dynein light-chain (LC8) peptides to target cytoplasmic dynein, see, e.g., Bergen et al., 2007, “Evaluation of an LC8-Binding Peptide for the Attachment of Artificial Cargo,” Mol Pharm 4(1): 119-128; and Xu et al., 2005, “A combination of mutations enhances the neurotropism of AAV2,” Virology, 341: 203-214).
(3) The HAD peptides used herein correspond to human protein and should be less immunogenic than synthetic peptides (e.g., used by Terwilliger, 2005, Virol 341:203; see also WO 2016/119150 A2); and should work in human subjects (unlike prior art AAV9 capsids containing randomized peptide populations selected in mice—See, e.g., Hordeaux et al., 2018 Mol Ther 26:664, “The Neurotropic Properties of AAV-PHP.B are limited to C57BL/6J Mice; Matsuzaki et al, 2018, Neurosci Lett 665: 182-188 “Intravenous administration of the AAV-PHP.B capsid fails to upregulate transduction efficiency in the marmoset brain”).

In particular, when a HAD peptide is engineered into AAV capsids such as AAV9, AAVrh10 and AAVrh20 (which display strong tropisms for the CNS), efficiency of delivery and delivery to the CNS is further enhanced. See also, Castle, et al., 2014, “Long-distance Axonal Transport of AAV9 is Driven by Dynein and Kinesin-2 and Is Trafficked in a Highly Motile Rab7-positive Compartment” _i Molecular Therapy, 22(3):554-566.

The HAD peptide can be inserted into an AAV capsid, for example at sites that allow surface exposure of the peptide, such as within variable surface-exposed loops, and, in other examples, sites described herein corresponding to VR-I, VR-IV or VR-VIII of AAV9. In some embodiments, rAAV vectors comprising a HAD peptide cross the blood-brain barrier and reach the CNS.

In some embodiments, a peptide from a mouse axonemal dynein (MAD) heavy chain tail is used. MAD heavy chain tail also provides neural-tissue homing domains from which peptides may be derived for insertion into AAV capsid proteins and for use in re-directing rAAVs to cross the blood-brain barrier and target CNS tissues (see also, Deverman et al., 2016, “Cre-dependent selection yields AAV variants for widespread gene transfer to the adult brain” Nat Biotechnology, 34(2):204-209).

In some embodiments, the neural tissue-homing domain comprises the amino acid sequence TLAVPFK (SEQ ID NO: 27); and the peptide insertion derived therefrom comprises or consists of the TLAVPFK (SEQ ID NO: 27) sequence. In some embodiments, the peptide insertion comprises or consists of four, five, or six consecutive amino acids from TLAVPFK (SEQ ID NO: 27). In particular embodiments, the capsid protein is an AAV9 capsid protein and the TLAVPFK (SEQ ID NO: 27) insertion occurs immediately after at least one of the amino acid residues 451 to 461. In particular embodiments, the TLAVPFK (SEQ ID NO: 27) insertion occurs after an amino acid residue I451, N452, G453, S454, G455, Q456, N457, Q458, Q459, T460, or L461 of the AAV9 capsid (SEQ ID NO: 118), and in certain embodiments is after S454 of the AAV9 capsid. In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8, serotype 9e (AAV9e), serotype rh10 (AAVrh10), serotype rh20 (AAVrh20), serotype rh39 (AAVrh39), serotype hu.37 (AAVhu.37), and serotype rh74 (AAVrh74, version 1 and 2) (see FIG. 8), and the TLAVPFK (SEQ ID NO: 27) peptide insertion occurs immediately after an amino acid residue in the AAV capsid that corresponds to one of the amino acid residues 451 to 461 of the AAV9 capsid. The alignment of these different AAV serotypes, as shown in FIG. 8, indicates corresponding amino acid residues in the different amino acid sequences. In some particular embodiments, the TLAVPFK (SEQ ID NO: 27) peptide insertion occurs immediately after one of the amino acid residues within: 450-459 of AAV1 capsid; 449-458 of AAV2 capsid; 449-459 of AAV3 capsid; 443-453 of AAV4 capsid; 442-445 of AAV5 capsid; 450-459 of AAV6 capsid; 451-461 of AAV7 capsid; 451-461 of AAV8 capsid; 451-461 of AAV9 capsid; 452-461 of AAV9e capsid; 452-461 of AAVrh10 capsid; 452-461 of AAVrh20 capsid; or 452-461 of AAVhu.37, in the sequences depicted in FIG. 8. In some embodiments, the TLAVPFK (SEQ ID NO: 27) peptide insertion occurs immediately after an amino acid residue corresponding 588 of AAV9 capsid protein (see FIG. 8), where said peptide insertion is surface exposed when the capsid protein is packaged as an AAV particle.

In some embodiments, the TLAVPFK (SEQ ID NO: 27) peptide insertion does not occur in any of the sites described in US 2015/0079038 to Deverman et al., particularly, but not limited to an insertion in the VR-VIII of the AAV capsid protein, more particularly is not inserted into the AAV capsid protein at a position corresponding to between amino acids 588 to 589 of AAV9 (SEQ ID NO: 118), or after one of the amino acids corresponding to amino acids 586 to 592 (including 587, 588, 589 or 590) of AAV9 (as depicted in FIG. 8). In other embodiments, the peptide insertion at any site in the capsid protein, does not comprise or consist of the peptide TLAVPFK (SEQ ID NO: 27), or of the peptide QAVRTSL (SEQ ID NO: 23), or of the peptide TLAGPFK (SEQ ID NO: 53). In other embodiments, the peptide insertion does not comprise or consist of the peptide TLAVPFK (SEQ ID NO: 27), or of the peptide QAVRTSL (SEQ ID NO: 23), or of the peptide TLAGPFK (SEQ ID NO: 53) inserted in the VR-VIII loop of the AAV capsid protein, more particularly is not inserted into AAV capsid protein at a position corresponding to between amino acids 588 to 589 of AAV9, or after one of the amino acids corresponding to amino acids 586 to 592 (including 587, 588, 589 or 590) of AAV9 (as depicted in FIG. 8).

### 5.2.2.2 rAAV-EPO Vectors

In certain embodiments, the peptide insertion is a peptide derived from regions of erythropoietin (EPO). The peptide, referred to herein as a “EPO peptide” may be a sequence of consecutive amino acids from an EPO domain that binds IRR but is not erythropoietic, or a conformation analog designed to mimic the three-dimensional structure of said domain. Recombinant AAV vectors comprising one or more EPO peptides, e.g., inserted into a surface-exposed loop of an AAV capsid coat, are referred to herein as a “rAAV-EPO vectors.”

Erythropoietin (EPO) is primarily made in the kidney and helps increase red blood cell production in response to hypoxia. It has been found that EPO also crosses the blood brain barrier, e.g., by receptor-mediated cytosis, being detected in the cerebro-spinal fluid following systemic administration of high doses. It also has been found that EPO exerts a protective effect on the CNS, in terms of reducing inflammation, preventing neuronal damage, and promoting repair (see, e.g., Cerami, 2001, “Beyond erythropoiesis: novel applications for recombinant human erythropoietin,” Semin Hematol. 38(3 Supp 7): 33-39). To reduce the deleterious side effect of erythropoiesis, and risk of thrombosis, however, non-erythropoeitic forms were developed, including ARA290. ARA290 is a nonerythropoietic analog of EPO, an 11 amino acid synthetic peptide, which binds Innate Repair Receptor (IRR), a receptor for EPO separate from the erythropoietic receptor that is expressed in response to hypoxia, injury, inflammation, or brain damage, and which exerts therapeutic effect in protecting brain tissue (see, e.g., Chen et al., 2013, “Therapeutic effects of nonerythropoietic erythropoietin analog ARA290 in experimental autoimmune encephalomyelitis rat,”J ofNeuroimmunology, 268:64-70; Collino, et al., 2015, “Flipping the molecular switch for innate protection and repair of tissues: Long-lasting effects of a non-erythropoietic small peptide engineered from erythropoietin,” Pharmacology & Therapeutics, 151:32-40; and Liu et al., 2014, “Erythropoietin-derived non-erythropoietic ameliorates experimental autoimmune neuritis by inflammation suppression and tissue protection,” PLOS One, 9(3): 1-10).

In some embodiments of the invention, the peptide insertion derived from EPO comprises at least 4 and up to 20 contiguous amino acids, and in certain embodiments no more than 12 contiguous amino acids, from the amino acid sequence of erythropoietin that is not erythropoietic and that binds Innate Repair Receptor (IRR); or a synthetic peptide modeled on 4-20 non-contiguous amino acids that form a conformation analog of erythropoietin that is not erythropoietic and that binds Innate Repair Receptor (IRR). In specific embodiments, the peptide insertion comprises at least 4 and up to 11 contiguous amino acids, and preferably 7 contiguous amino acids, from the synthetic peptide “ARA290,” having amino acid sequence QEQLERALNSS (SEQ ID NO: 8). In certain embodiments, the peptide insertion comprises or consists of the ARA290 sequence QEQLERALNSS (SEQ ID NO: 8). In some embodiments, the EPO peptide comprises or consists of hyposialated EPO (hsEPO), or hsEPO with one or more amino acid modifications to increase its serum half life

The EPO peptide can be inserted into an AAV capsid, for example at sites that allow surface exposure of the peptide, such as within variable surface-exposed loops, and, in more examples, sites described herein in an AAV capsid protein corresponding to VR-I, VR-IV or VR-VIII of AAV9 or may be inserted after the first amino acid of VP2, e.g. immediately after amino acid 137 (AAV4, AAV4-4, and AAV5) or immediately after amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). In some embodiments, rAAV vectors comprising an EPO peptide cross the blood-brain barrier and reach the CNS. Use of EPO peptides in rAAVs provides the additional advantage of reducing inflammation in at least two ways. First, by binding IRR, rAAV-EPO vectors trigger a subject's anti-inflammatory response, thus off-setting inflammation that may result from introduction of foreign agents (the rAAV vectors) to the subject. Second, ARA290 has been known for having a relatively short half-life, which provides the advantage of rapid clearance, and thus reduced time to trigger inflammation.

5.2.2.3 rAAV-SRL Vectors

In certain embodiments, the peptide insertion is a peptide derived from regions of brain-homing domains having an SRL (serine-arginine-lysine) motif. The peptide, referred to herein as a “SRL peptide” may be a sequence of consecutive amino acids from a domain having an SRL motif that targets brain tissue, or a conformation analog designed to mimic the three-dimensional structure of said domain. Recombinant AAV vectors comprising one or more SRL peptides, e.g., inserted into a surface-exposed loop of an AAV capsid coat, are referred to herein as “rAAV-SRL vectors.”

A family of brain-homing peptides has been reported, where each peptide in the family contains the common amino acid motif, SRL (serine-arginine-leucine), but different flanking amino acid sequences (see, e.g., U.S. Pat. No. 5,622,699). In some embodiments, the peptide insertion from said brain-homing domain comprises at least 4, 5, 6, 7, 8 or all 9 amino acids from sequence CLSSRLDAC (SEQ ID NO: 11), particularly including the SRL motif. In some embodiments, the peptide insertion comprises or consists of the sequence CLSSRLDAC (SEQ ID NO: 11).

It has been found that both of the cysteine residues in certain homing peptides can be deleted without significantly affecting the organ homing activity of the peptide. For example, a peptide having the sequence LSSRLDA (SEQ ID NO: 10) also can be a brain-homing peptide. Methods for determining the necessity of a cysteine residue or of amino acid residues N-terminal or C-terminal to a cysteine residue for organ homing activity of a peptide are routine and well known in the art. Thus, in some embodiments, the peptide insertion comprises at least 4, 5, 6, or all 7 amino acids from sequence LSSRLDA (SEQ ID NO: 10). In some embodiments, the peptide insertion comprises or consists of the sequence LSSRLDA (SEQ ID NO: 10).

The SRL peptide can be inserted into an AAV capsid, for example at sites that allow surface exposure of the peptide, such as within variable surface-exposed loops, and, in more examples, sites described herein corresponding to VR-I, VR-IV, or VR-VIII of AAV9 or may be inserted after the first amino acid of VP2, e.g. immediately after amino acid 137 (AAV4, AAV4-4, and AAV5) or immediately after amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). In some embodiments, rAAV vectors comprising an SRL peptide cross the blood-brain barrier and reach the CNS.

### 5.2.3 Cytoplasmic Dynein-Homing Peptides

Another aspect of the present invention relates to capsid proteins comprising peptide insertions designed to confer or enhance homing to cytoplasmic dynein. Examples include peptides derived from regions of cytoplasmic dynein-homing domains, such as a dynein light chain-homing domain (see, e.g., Midoux, et al., 2017, “Peptides mediating DNA transport on microtubules and their impact on non-viral gene transfer efficiency,” Bioscience Reports (review article), 37 BSR20170995). The peptide, referred to herein as a “cytoplasmic dynein-homing peptide” may be a sequence of consecutive amino acids from a cytoplasmic dynein-homing region of a protein, or a conformation analog designed to mimic the three-dimensional structure thereof. These peptides include SITLVKSTQTV (SEQ ID NO: 21) (alternatively, CITLVKSTQTV (SEQ ID NO: 54)), TILSRSTQTG (SEQ ID NO: 22), VVMVGEKPITITQHSVETEG (SEQ ID NO: 25), RSSEEDKSTQTT (SEQ ID NO: 26), KSTEDKSTQTP (SEQ ID NO: 46); LGHFTRSTQTS (SEQ ID NO: 47); GVQMAKSTQTF (SEQ ID NO: 48); PKTRNSQTQTD (SEQ ID NO: 49); VTTQNTASQTM (SEQ ID NO: 50); and KSSQDKSTQTTGD (SEQ ID NO: 51). Peptides or domains of proteins that associate with the light chain of cytoplasmic dynein may have the motif TQT (threonine-glutamine-threonine) or STQT (serine-threonine-glutamine-threonine) (SEQ ID NO: 55) or even KSTQT (lysine-serine-threonine-glutamine-threonine) (SEQ ID NO: 56). Accordingly, in certain embodiments, the cytoplasmic dynein-homing peptide is a portion of a peptide which contains the TQT, STQT (SEQ ID NO: 55) or KSTQT (SEQ ID NO: 56) motif and has the cytoplasmic dynein-homing activity.

In some embodiments, the peptide insertion from said dynein light-chain homing domain comprises at least 4, 5, 6, 7, 8, 9, 10, or all 11 consecutive amino acids of sequence SITLVKSTQTV (SEQ ID NO: 21), preferably which contains the TQT, STQT (SEQ ID NO: 55) or KSTQT (SEQ ID NO: 56) motif and/or has the cytoplasmic dynein-homing activity. In some embodiments, the peptide insertion consists of at least 4, 5, 6, 7, 8, 9, 10, or all 11 consecutive amino acids of sequence SITLVKSTQTV (SEQ ID NO: 21), preferably which contains the TQT, STQT (SEQ ID NO: 55) or KSTQT (SEQ ID NO: 56) motif and/or has the cytoplasmic dynein-homing activity.

In some embodiments, the peptide insertion from said dynein light-chain homing domain comprises at least 4, 5, 6, 7, 8, 9, or all 10 consecutive amino acids of sequence TILSRSTQTG (SEQ ID NO: 22), preferably which contains the TQT or STQT (SEQ ID NO: 55) motif and/or has the cytoplasmic dynein-homing activity. In some embodiments, the peptide insertion consists of at least 4, 5, 6, 7, 8, 9, or all 10 consecutive amino acids of sequence TILSRSTQTG (SEQ ID NO: 22), preferably which contains the TQT or STQT (SEQ ID NO: 55) motif and/or has the cytoplasmic dynein-homing activity.

In some embodiments, the peptide insertion from said dynein light-chain homing domain comprises at least 4 and up to all 20 consecutive amino acids of sequence VVMVGEKPITITQHSVETEG (SEQ ID NO: 25). In some embodiments, the peptide insertion consists of at least 4 and up to all 20 consecutive amino acids of sequence VVMVGEKPITITQHSVETEG (SEQ ID NO: 25). In some embodiments, the peptide insertion comprises or consists of 7, 8, 9, 10, 11, 12, 13, or 14 or 15 consecutive amino acids of sequence VVMVGEKPITITQHSVETEG (SEQ ID NO: 25).

In some embodiments, the peptide insertion from said dynein light-chain homing domain comprises at least 4, 5, 6, 7, 8, 9, 10, 11, or all 12 consecutive amino acids of sequence RSSEEDKSTQTT (SEQ ID NO: 26), preferably which contains the TQT, STQT (SEQ ID NO: 55) or KSTQT (SEQ ID NO: 56) motif and/or has the cytoplasmic dynein-homing activity. In some embodiments, the peptide insertion consists of at least 4, 5, 6, 7, 8, 9, 10, 11, or 12 consecutive amino acids of sequence RSSEEDKSTQTT (SEQ ID NO: 26), preferably which contains the TQT, STQT (SEQ ID NO: 55) or KSTQT (SEQ ID NO: 56) motif and/or has the cytoplasmic dynein-homing activity.

In some embodiments, the peptide insertion from said dynein light-chain homing domain comprises at least 4, 5, 6, 7, 8, 9, 10, 11, or 12 consecutive amino acids of one of the peptides having the sequence KSTEDKSTQTP (SEQ ID NO: 46); LGHFTRSTQTS (SEQ ID NO: 47); GVQMAKSTQTF (SEQ ID NO: 48); PKTRNSQTQTD (SEQ ID NO: 49); VTTQNTASQTM (SEQ ID NO: 50); or KSSQDKSTQTTGD (SEQ ID NO: 51), preferably which contains the TQT, STQT (SEQ ID NO: 55) or KSTQT (SEQ ID NO: 56) motif and/or has the cytoplasmic dynein-homing activity. In some embodiments, the peptide insertion consists of at least 4, 5, 6, 7, 8, 9, 10, 11, 12 or 13 consecutive amino acids of one of the peptides having the sequence KSTEDKSTQTP (SEQ ID NO: 46); LGHFTRSTQTS (SEQ ID NO: 47); GVQMAKSTQTF (SEQ ID NO: 48); PKTRNSQTQTD (SEQ ID NO: 49); VTTQNTASQTM (SEQ ID NO: 50); or KSSQDKSTQTTGD (SEQ ID NO: 51), preferably which contains the TQT, STQT (SEQ ID NO: 55) or KSTQT (SEQ ID NO: 56) motif and/or has the cytoplasmic dynein-homing activity.

The cytoplasmic dynein-homing peptide can be inserted into an AAV capsid, for example, at sites that allow surface exposure of the peptide, such as within variable surface-exposed loops, and, in more examples, sites described herein corresponding to VR-IV or VIII of AAV9 or may be inserted after the first amino acid of VP2, e.g. immediately after amino acid 137 (AAV4, AAV4-4, and AAV5) or immediately after amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8).

### 5.2.4 Bone-Homing Peptides

Another aspect of the present invention relates to capsid proteins comprising peptide insertions designed to confer or enhance bone-homing properties. Examples include peptides from a bone-binding domain of a protein, or a conformational analog of said domain. A peptide from a bone-binding or bone-homing domain is referred to as a bone-homing peptide (bone tissue-homing or bone-cell or cell-matrix-homing).

In certain embodiments, the peptide insertion may be a sequence of consecutive amino acids from a HA-binding domain that targets bone tissue, or a conformation analog designed to mimic the three-dimensional structure of said domain. For example, a six to eight residue stretch of L-Asp has been shown to enhance targeting of an enzyme to hydroxyapatite (see, e.g., Nishioka, et al., 2006, “Enhancement of drug delivery to bone: Characterization of human tissue-nonspecific alkaline phosphatase tagged with an acidic oligopeptide,”Mol Genet Metab. 88(3):244-255; and Kasugai, et al., 2000, “Selective drug delivery system to bone: small peptide (Asp)6 (SEQ ID NO: 57) conjugation,” J Bone Miner Res. 15(5):936-943).

In particular embodiments, the peptide insertion from said HA-binding domain comprises at least 4, 5, 6, 7, or all 8 amino acids from sequence DDDDDDDD (SEQ ID NO: 9). In some embodiments, the peptide insertion consists of at least 4, 5, 6, 7, or all 8 amino acids from sequence DDDDDDDD (SEQ ID NO: 9). In a particular embodiment, the peptide insertion comprises or consists of the DDDDDDDD (SEQ ID NO: 9) sequence.

The bone-homing peptide can be inserted into an AAV capsid, for example at sites that allow surface exposure of the peptide, such as within variable surface-exposed loops, and, in more examples, sites described herein in an AAV capsid protein corresponding to VR-I, VR-IV, or VR-VIII of AAV9 or may be inserted after the first amino acid of VP2, that is immediately after amino acid 137 (AAV4, AAV4-4, and AAV5) or immediately after amino acid 138 (AAV1, AAV3, AAV3-3, AAV6, AAV7, AAV8, AAV9, AAV9e, rh.10, rh.20, rh.39, rh.74v1, rh.74v2, and hu.37) (FIG. 8). Recombinant AAV vectors comprising one or more bone-homing peptides, e.g., inserted into a surface-exposed loop of an AAV capsid coat, are referred to herein as “rAAV bone-homing vectors.” In particular embodiments, the capsid protein is an AAV9 capsid protein and the bone-homing insertion occurs immediately after at least one of the amino acid residues 451 to 461 of the AAV9 capsid or immediately after amino acid 138. In other embodiments, the capsid protein is from at least one AAV type selected from AAV1, AAV3, AAV4, AAV5, AAV6, AAV7, AAV8, AAVrh8, AAV9, AAV9e, AAVrh10, AAVrh20, AAVhu.37, AAVrh39, and AAVrh74 (verisons 1 and 2) (see FIG. 8), and the bone-homing peptide insertion occurs immediately after an amino acid residue corresponding to at least one of the amino acid residues 451 to 461 of AAV9. The alignments of these different AAV serotypes, as shown in FIG. 8, indicates corresponding amino acid residues in the different amino acid sequences.

### 5.2.5 Kidney-Homing Peptides

Another aspect of the present invention relates to capsid proteins comprising peptide insertions designed to confer or enhance kidney-homing properties, including homing to kidney tissue, kidney cells or kidney cell matrix. Examples include peptides from a kidney-binding domain of a protein, or a conformational analog of said domain. A peptide from a kidney-binding or kidney-homing domain is referred to as a kidney-homing peptide. In certain embodiments, the kidney-homing peptide preferentially targets the kidney as compared to the liver, and relative to an AAV that has not been engineered to contain the kidney-homing peptide.

In certain embodiments, the peptide insertion may be a sequence of consecutive amino acids from a domain that targets kidney tissue, or a conformation analog designed to mimic the three-dimensional structure of said domain. In some embodiments, the kidney-homing domain comprises the sequence CLPVASC (SEQ ID NO: 12) (see, e.g., U.S. Pat. No. 5,622,699). In some embodiments, the peptide insertion from said kidney-homing domain comprises at least 4, 5, 6, or all 7 amino acids from sequence CLPVASC (SEQ ID NO: 12). In some embodiments, the peptide insertion comprises or consists of the sequence CLPVASC (SEQ ID NO: 12).

It has been found that both of the cysteine residues in certain homing peptides can be deleted without significantly affecting the organ homing activity of the peptide. For example, a peptide having the sequence LPVAS (SEQ ID NO: 13) also can be a kidney-homing peptide. Methods for determining the necessity of a cysteine residue or of amino acid residues N-terminal or C-terminal to a cysteine residue for organ homing activity of a peptide are routine and well known in the art. Thus, in some embodiments, the peptide insertion comprises at least 4 or all 5 amino acids from sequence LPVAS (SEQ ID NO: 13). In some embodiments, the peptide insertion comprises or consists of the sequence LPVAS (SEQ ID NO: 13).

The kidney-homing peptide can be inserted into an AAV capsid, for example, at sites that allow surface exposure of the peptide, such as within variable surface-exposed loops, and, in more examples, sites described herein corresponding to VR-I, VR-IV or VR-VIII of AAV9 or may be inserted after the first amino acid of VP2, e.g. immediately after amino acid 137 (AAV4, AAV4-4, and AAV5) or immediately after amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). Recombinant AAV vectors comprising one or more kidney-homing peptides, e.g., inserted into a surface-exposed loop of an AAV capsid coat, are referred to herein as “rAAV kidney-homing vectors.” In particular embodiments, the capsid protein is an AAV9 capsid protein and the kidney-homing peptide insertion occurs immediately after at least one of the amino acid residues 451 to 461 of the AAV9 capsid. In other embodiments, the capsid protein is from at least one AAV type selected from AAV1, AAV3, AAV4, AAV5, AAV6, AAV7, AAV8, AAVrh8, AAV9, AAV9e, AAVrh10, AAVrh20, AAVrh39, AAVhu.37, and AAVrh74 (version 1 and 2) (see FIG. 8), and the kidney-homing peptide insertion occurs immediately after an amino acid residue corresponding to at least one of the amino acid residues 451 to 461 of AAV9. The alignments of these different AAV serotypes, as shown in FIG. 8, indicates corresponding amino acid residues in the different amino acid sequences.

5.2.6 Muscle-Homing Peptides

Another aspect of the present invention relates to capsid proteins comprising peptide insertions designed to confer or enhance muscle-homing properties, including homing to muscle tissue, muscle cells or muscle cell matrix. Examples include peptides from a muscle-binding domain of a protein, or a conformational analog of said domain. A peptide from a muscle-binding or muscle-homing domain is referred to as a muscle-homing peptide.

In certain embodiments, the peptide insertion may be a sequence of consecutive amino acids from a domain that targets muscle, or a conformation analog designed to mimic the three-dimensional structure of said domain. In some embodiments, the muscle-homing domain comprises the sequence ASSLNIA (SEQ ID NO: 14) (see, e.g., Samoylov, et al., 2002, “Recognition of cell-specific binding of phage display derived peptides using an acoustic wave sensor,” Biomol Eng, 18(6):269-272). In some embodiments, the peptide insertion from said muscle-homing domain comprises at least 4, 5, 6, or all 7 amino acids from sequence ASSLNIA (SEQ ID NO: 14). In some embodiments, the peptide insertion comprises or consists of the sequence ASSLNIA (SEQ ID NO: 14).

The muscle-homing peptide can be inserted into an AAV capsid, for example, at sites that allow surface exposure of the peptide, such as within variable surface-exposed loops, and, in more examples, sites described herein corresponding to VR-I, VR-IV, or VR-VIII of AAV9 or may be inserted after the first amino acid of VP2, e.g. after amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). Recombinant AAV vectors comprising one or more muscle-homing peptides, e.g., inserted into a surface-exposed loop of an AAV capsid coat, are referred to herein as “rAAV muscle-homing vectors.” In particular embodiments, the capsid protein is an AAV9 capsid protein and the muscle homing peptide insertion occurs immediately after at least one of the amino acid residues 451 to 461 of the AAV9 capsid. In other embodiments, the capsid protein is from at least one AAV type selected from AAV1, AAV3, AAV4, AAV5, AAV6, AAV7, AAV8, AAVrh8, AAV9, AAV9e, AAVrh10, AAVrh20, AAVhu.37, AAVrh39, and AAVrh74 (versions 1 and 2) (see FIG. 8), and the muscle-homing peptide insertion occurs immediately after an amino acid residue corresponding to at least one of the amino acid residues 451 to 461 of AAV9 (SEQ ID NO: 118). The alignments of these different AAV serotypes, as shown in FIG. 8, indicates corresponding amino acid residues in the different amino acid sequences.

### 5.2.7 TfR-Homing Peptides

Another aspect of the present invention relates to capsid proteins comprising peptide insertions designed to confer or enhance homing properties to transferrin receptors. Examples include peptides from transferrin receptor-binding domains of a protein, or a conformational analog of said domain. A peptide from a transferrin receptor-binding or transferrin receptor-homing domain is referred to as a transferrin receptor-homing peptide.

The human transferrin receptor (hTfR) has been studied as a model for receptor-mediated endocytosis and as a marker for cellular proliferation. The hTfR generally is highly expressed in proliferative cells (such as tumor cells), has being over-expressed at least 100-fold in oral, liver, pancreatic, and prostate cancer. This makes hTfR a useful diagnostic marker as well as a target for cancer therapies. The TfR also is expressed on the blood brain barrier. TfR is a dimer composed of two identical 95 kDa subunits and is responsible for iron uptake by a cell. Iron is carried in the blood by 80 kDa transferrin (Tf), which binds TfR to form a complex that is internalized through clathrin-coated pits. Iron is released from transferrin in the acidic region of the endosome, leaving an apotransferrin-receptor complex, which is recycled back to the cell surface and the apotransferrin (transferrin not bound to iron) also is recycled. See, e.g., Cheng, et al., 2004, “Structure of the human transferrin receptor-transferrin complex,” Cell 116(4): 565-576.

As transferrin receptors are involved in receptor-mediated transcytosis, they may serve as a “Trojan horse” in delivering cargo across the blood brain barrier, such as in delivering small molecule drugs, enzymes, or nucleic acid molecules. For example, studies in mice have shown uptake of engineered TfR-binding peptides by CEF cells that express TfR, facilitating entry into brain parenchyma via brain micro vessels over time (see, Lee et al., The FEBS Journal, 2001; and Staquicini et al, 2011, “Systemic combinatorial peptide selection yields a non-canonical iron-mimicry mechanism for targeting tumors in a mouse model of human glioblastoma,” J. of Clinical Investigation, 121(1):161-173).

In some embodiments, the TfR peptide insertion provides enhanced transport of AAV particles encapsidating a transgene across an endothelial cellular matrix.

In certain embodiments, the peptide insertion may be a sequence of consecutive amino acids from a Tf domain that binds the TfR, or a conformation analog designed to mimic the three-dimensional structure of said domain, or an iron-mimic. In some embodiments, the peptide insertion from the TfR-homing domain comprises 4, 5, 6, or all 7 amino acids from sequence HAIYPRH (SEQ ID NO: 17), or consists of the sequence HAIYPRH (SEQ ID NO: 17). In some embodiments, the peptide insertion from the TfR-homing domain comprises 4, 5, 6, 7, 8, 9, 10, 11, or all 12 amino acids from sequence THRPPMWSPVWP (SEQ ID NO: 18) or consists of the sequence THRPPMWSPVWP (SEQ ID NO: 18) (see also, US 2006/0193778).

In some embodiments, the peptide insertion from the TfR-homing domain comprises 4, 5, 6, 7, 8, or all 9 amino acids from sequence CRTIGPSVC (SEQ ID NO: 20). In some embodiments, the peptide insertion comprises or consists of the sequence CRTIGPSVC (SEQ ID NO: 20). It has been found that both of the cysteine residues in certain homing peptides can be deleted without significantly affecting the organ homing activity of the peptide and methods for determining the necessity of a cysteine residue or of amino acid residues N-terminal or C-terminal to a cysteine residue for organ homing activity of a peptide are routine and well known in the art. In some embodiments, the peptide insertion comprises at least 4, 5, 6, or all 7 amino acids from sequence RTIGPSV (SEQ ID NO: 19). In some embodiments, the peptide insertion comprises or consists of the sequence RTIGPSV (SEQ ID NO: 19).

The TfR-homing peptide can be inserted into an AAV capsid, for example, at sites that allow surface exposure of the peptide, such as within variable surface-exposed loops, and, in more examples, sites described herein corresponding to VR-I, VR-IV, or VR-VIII of AAV9 or may be inserted after the first amino acid of VP2, e.g. after amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). Recombinant AAV vectors comprising one or more TfR -homing peptides, e.g., inserted into a surface-exposed loop of an AAV capsid coat, are referred to herein as “rAAV TfR-homing vectors.”

In some embodiments, the TfR-homing domain comprises the amino acid sequence RTIGPSV (SEQ ID NO: 19); and the peptide insertion derived therefrom comprises or consists of the RTIGPSV (SEQ ID NO: 19) or CRTIGPSVC (SEQ ID NO: 20) sequence. In some embodiments, the peptide insertion comprises or consists of 4, 5, 6, or all 7 consecutive amino acids from RTIGPSV (SEQ ID NO: 19); or comprises or consists of 4, 5, 6, 7, 8, or all 9 amino acids from CRTIGPSVC (SEQ ID NO: 20). In particular embodiments, the capsid protein is an AAV9 capsid protein and the RTIGPSV (SEQ ID NO: 19) or CRTIGPSVC (SEQ ID NO: 20) insertion occurs immediately after at least one of the amino acid residues 451 to 461. In particular embodiments, the RTIGPSV (SEQ ID NO: 19) or CRTIGPSVC (SEQ ID NO: 20) insertion occurs after an amino acid residue selected from the group consisting of I451, N452, G453, S454, G455, Q456, N457, Q458, Q459, T460, and L461 of the AAV9 capsid. In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8, serotype 9e (AAV9e), serotype rh10 (AAVrh10), serotype rh20 (AAVrh20), serotype hu37(AAVhu.37), serotype rh39 (AAVrh39), and serotype rh74 (AAVrh74, version 1 and 2) (see FIG. 8) and the RTIGPSV (SEQ ID NO: 19) or CRTIGPSVC (SEQ ID NO: 20) peptide insertion occurs immediately after an amino acid residue corresponding to at least one of the amino acid residues 451 to 461. The alignments of these different AAV serotypes, as shown in FIG. 8, indicates corresponding amino acid residues in the different amino acid sequences. In some particular embodiments, the RTIGPSV (SEQ ID NO: 19) or CRTIGPSVC (SEQ ID NO: 20) peptide insertion occurs immediately after one of the amino acid residues within: 450-459 of AAV1 capsid (SEQ ID NO: 110); 449-458 of AAV2 capsid (SEQ ID NO: 111); 449-459 of AAV3 capsid (SEQ ID NO: 112); 443-453 of AAV4 capsid (SEQ ID NO: 113); 442-445 of AAV5 capsid (SEQ ID NO: 114); 450-459 of AAV6 capsid (SEQ ID NO: 115); 451-461 of AAV7 capsid (SEQ ID NO: 116); 451-461 of AAV8 capsid (SEQ ID NO: 117); 451-461 of AAV9 capsid (SEQ ID NO: 118); 452-461 of AAV9e capsid (SEQ ID NO: 119); 452-461 of AAVrh10 capsid (SEQ ID NO: 120); 452-461 of AAVrh20 capsid (SEQ ID NO: 121); 452-461 of AAVhu.37 (SEQ ID NO: 122); 452-461 of AAVrh74 capsid (SEQ ID NO: 123 or SEQ ID NO: 154); or 452-461 of AAVrh39 capsid (SEQ ID NO: 124); in the sequences depicted in FIG. 8. In some embodiments, the RTIGPSV (SEQ ID NO: 19) or CRTIGPSVC (SEQ ID NO: 20) peptide insertion occurs immediately after an amino acid residue corresponding to 588 of AAV9 capsid protein (see FIG. 8), where said peptide insertion is surface exposed when the capsid protein is packaged as an AAV particle.

5.2.8 Retinal Cell-Homing Peptides

Another aspect relates to capsid proteins comprising peptide insertions designed to confer or enhance retinal cell-homing properties. Examples include peptides from a retinal cell-binding domain of a protein, or a conformational analog of said domain. A peptide from a retinal cell-binding or retinal cell-homing domain is referred to as a retinal cell-homing peptide. The term “retinal cell” refers to one or more of the cell types found in or near the retina, including amacrine cells, bipolar cells, horizontal cells, Muller glial cells, photoreceptor cells (e.g., rods and cones), retinal ganglion cells, retinal pigmented epithelium, and the like, and in particular, human photoreceptor cells (e.g., human cone cells and/or human rod cells), human horizontal cells, human bipolar cells, human amacrine cells, as well as human retina ganglion cells (e.g., midget cells, parasol cells, bistratified cells, giant retina ganglion cells, photosensitive ganglion cells, and/or Muller glia), endothelial cells in the inner limiting membrane, and/or human retinal pigment epithelial cells in the external limiting membrane.

In certain embodiments, the peptide insertion may be a sequence of consecutive amino acids from a retinal cell-binding domain that targets retinal tissue, or a conformation analog designed to mimic the three-dimensional structure of said domain.

In particular embodiments, the peptide insertion is a peptide derived from regions of human axonemal dynein (HAD) heavy chain tail. As noted above, the peptide referred to herein as a “HAD peptide” may be a sequence of consecutive amino acids from HAD heavy chain tail region, or a conformation analog designed to mimic the three-dimensional structure thereof. Table 2, provided above, identifies the tail and dimerization domain of the human axonemal dyneins, as well as peptides for use as peptide insertions in the engineered capsid proteins described herein, including for use as retinal cell-homing peptides. In some embodiments, insertions of at least 4 and up to 15 contiguous amino acids, and preferably 7 contiguous amino acids, from the axonemal dynein sequences of the stem/tail region and/or the dimerization domain (NDD) are used as the peptide insertion for targeting retinal cells.

In some embodiments, the peptide for insertion in an AAV capsid is designed from the dimerization domain (NDD) of a HAD heavy chain tail region. In alternate embodiments, peptides corresponding to the amino acid sequences of the remainder of the HAD heavy chain tail (i.e., excluding the dynein motor domain) can be used. In some embodiments, the peptide insertion comprises at least 4, in an embodiment is 7 contiguous amino acids, and is up to 12 or 15 contiguous amino acids from a dimerization domain of a HAD heavy chain tail. In particular embodiments, the peptide insertion comprises at least 4, is 7 contiguous amino acids, and is up to 12 or 15 contiguous amino acids from the group consisting of (depicted in FIGS. 7A-7M): amino acids (“aa”) 1-1542 of DYH1_HUMAN UniProtKB-Q9P2D7 (SEQ ID NO. 97); aa 1-1764 of DYH2_HUMAN UniProtKB-Q9P225 (SEQ ID NO. 98); aa 1-1390 of DYH3_HUMAN UniProtKB-Q8TD57 (SEQ ID NO. 99); aa 1-1941 of DYH5_HUMAN UniProtKB-Q8TE73 (SEQ ID NO. 100); aa 1-1433 of DYH6_HUMAN UniProtKB-Q9C0G6 (SEQ ID NO. 101); aa 1-1289 of DYH7_HUMAN UniProtKB-Q8WXX0(SEQ ID NO. 102); aa 1-1807 of DYH8_HUMAN UniProtKB-Q96JB1 (SEQ ID NO. 3); aa 1-1831 of DYH9_HUMAN UniProtKB-Q9NYC9 (SEQ ID NO. 104); aa 1-1793 of DYH10_HUMAN UniProtKB-Q8IVF4 (SEQ ID NO. 105); aa 1-1854 of DYH11_HUMAN UniProtKB-Q96DT5 (SEQ ID NO. 106); aa 1-1214 of DYH12_HUMAN UniProtKB-Q6ZR08 (SEQ ID NO. 107); aa 1-200 of DYH14_HUMAN UniProtKB-Q0DD8 (SEQ ID NO. 108); and aa 1-1794 of DYH17_HUMAN UniProtKB-Q9UFH2 (SEQ ID NO. 109)) and is used to target engineered AAVs to retinal cells. In more preferred embodiments, the peptide insertion comprises at least 4 contiguous amino acids, is 7 contiguous amino acids, and is up to 12 or 15 contiguous amino acids from residues 1-200 of any one of the dynein heavy chain sequences recited above, that is, any one from the group consisting of amino acids (“aa”) 1-1542 of DYH1_HUMAN UniProtKB-Q9P2D7 (SEQ ID NO. 97); aa 1-1764 of DYH2_HUMAN UniProtKB Q9P225 (SEQ ID NO. 98); aa 1-1390 of DYH3_HUMAN UniProtKB-Q8TD57 (SEQ ID NO. 99); aa 1-1941 of DYH5_HUMAN UniProtKB-Q8TE73 (SEQ ID NO. 100); aa 1-1433 of DYH6_HUMAN UniProtKB-Q9C0G6 (SEQ ID NO. 101); aa 1-1289 of DYH7_HUMAN UniProtKB-Q8WXX0(SEQ ID NO. 102);; aa 1-1807 of DYH8_HUMAN UniProtKB-Q96JB1 (SEQ ID NO. 3); aa 1-1831 of DYH9_HUMAN UniProtKB-Q9NYC9 (SEQ ID NO. 104); aa 1-1793 of DYH10_HUMAN UniProtKB-Q8IVF4 (SEQ ID NO. 105); aa 1-1854 of DYH11_HUMAN UniProtKB-Q96DT5 (SEQ ID NO. 106); aa 1-1214 of DYH12_HUMAN UniProtKB-Q6ZR08 (SEQ ID NO. 107); aa 1-200 of DYH14_HUMAN UniProtKB-Q0VDD8 (SEQ ID NO. 108); and aa 1-1794 of DYH17_HUMAN UniProtKB Q9UFH2 (SEQ ID NO. 109)) for targeting retinal cells. In still more preferred embodiments, the peptide insertion is 7 contiguous amino acids from any one of the dynein heavy chain sequences of FIGS. 7A-7M; or is 7 contiguous amino acids from residues 1-200 of any one of the dynein heavy chain sequences (FIGS. 7A-7M) and is used to target engineered AAVs to retinal cells.

In particular embodiments, the peptide insertion for targeting retinal cells is at least or consists of 4, 5, 6, or 7 contiguous amino acids from the group consisting of: KMQVPFQ (SEQ ID NO: 1); TLAAPFK (SEQ ID NO: 2); QQAAPSF (SEQ ID NO: 3); RYNAPFK (SEQ ID NO: 4); LKLPPIV (SEQ ID NO: 5); PFIKPFE (SEQ ID NO: 6); and TLSLPWK (SEQ ID NO: 7). In still more particular embodiments, the peptide insertion for targeting retinal cells consists of a peptide from the group consisting of: KMQVPFQ (SEQ ID NO: 1); TLAAPFK (SEQ ID NO: 2); QQAAPSF (SEQ ID NO: 3); RYNAPFK (SEQ ID NO: 4); LKLPPIV (SEQ ID NO: 5); PFIKPFE (SEQ ID NO: 6); and TLSLPWK (SEQ ID NO: 7). In one embodiment of particular interest, the peptide insertion comprises or consists of the amino acid sequence TLAAPFK (SEQ ID NO: 2).

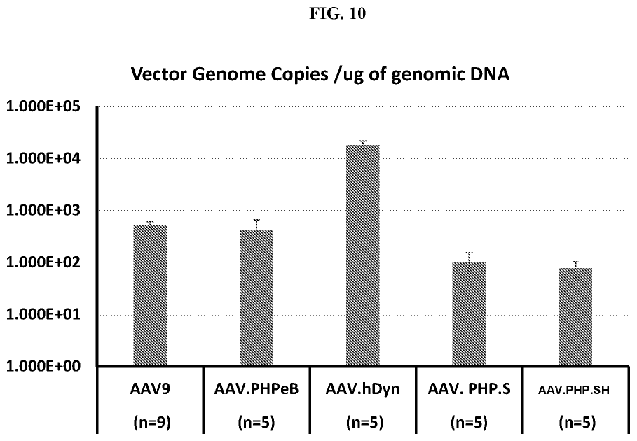

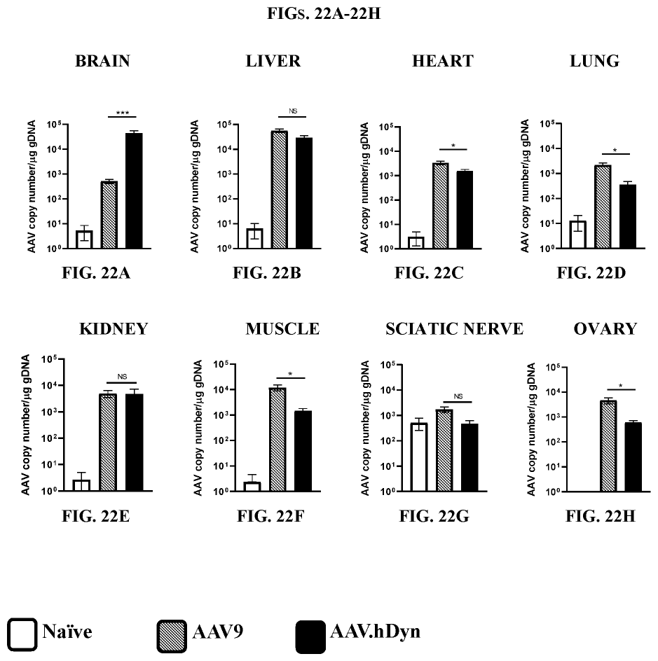

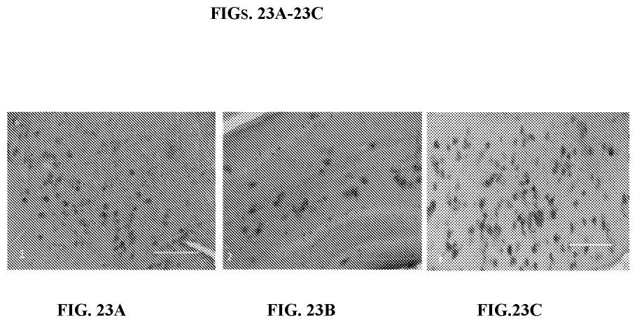

The HAD peptide can be inserted into an AAV capsid, for example at sites that allow surface exposure of the peptide, such as within variable surface-exposed loops, and, in more examples, sites described herein corresponding to VR-I, VR-IV or VR-VIII of AAV9 or may be inserted after the first amino acid of VP2, e.g. immediately after amino acid 137 (AAV4, AAV4-4, and AAV5) or immediately after amino acid 138 (AAV1, AAV2, AAV3, AAV3-3, AAV6, AAV7, AAV8, AAV9, AAV9e, rh.10, rh.20, rh.39, rh.74v1, rh.74v2, and hu.37) (FIG. 8). In some embodiments, rAAV vectors comprising a HAD peptide is used to target cells of the retina. In a particular embodiment, a capsid protein of AAV9 with TLAAPFK (SEQ ID NO: 2) between amino acids 588-589 (SEQ ID NO: 118) is used to target retinal cell (see, e.g., the vector used in FIG. 10, FIGS. 22A-22H, and FIGS. 23A-23C). In some embodiment, a capsid protein of a different AAV is used for targeting retinal cells, where the vector includes TLAAPFK (SEQ ID NO: 2) between amino acids corresponding to amino acids 588-589 of AAV9 (see again FIG. 8).

In preferred embodiments, the retinal cell-homing peptide causes the AAV to transduce retinal cells following local administration, such as intravitreal injection. In more preferred embodiments, the retinal cell-homing peptide causes the AAV to transduce retinal cells following systemic administration, such as intravenous injection. In most preferred embodiments, the engineered AAV for targeting and transducing retinal cells comprises a capsid protein of AAV9 with TLAAPFK (SEQ ID NO: 2) between amino acids 588-589 of SEQ ID NO: 118.

In some embodiments, the peptide insertion from a retinal cell-binding domain comprises at least 4, 5, 6, 7, 8, or all 9 amino acids from sequence LALGETTRP (SEQ ID NO: 16). In some embodiments, the peptide insertion consists of at least 4, 5, 6, 7, 8, or all 9 amino acids from sequence LALGETTRP (SEQ ID NO: 16). In some embodiments, the peptide insertion comprises at least 4, 5, 6, or all 7 amino acids from sequence LGETTRP (SEQ ID NO: 15). In particular embodiments, the peptide insertion consists of at least 4, 5, 6, or all 7 amino acids from sequence LGETTRP (SEQ ID NO: 15). In a particular embodiment, the peptide insertion consists of the LGETTRP (SEQ ID NO: 15) sequence.

The retinal cell-homing peptide can be inserted into an AAV capsid, preferably at sites that allow surface exposure of the peptide, such as within variable surface-exposed loops, and, more preferably, sites described herein corresponding to VR-I, VR-IV, or VR-VIII of AAV9, or in the corresponding position of AAV8. In particular embodiments, the capsid protein is an AAV8 capsid protein and the LGETTRP (SEQ ID NO: 15) or LALGETTRP (SEQ ID NO: 16) insertion occurs immediately after at least one of the amino acid residues 451 to 461 of the AAV8 capsid (amino acid sequence of SEQ ID NO: 117). In particular embodiments, the capsid protein is an AAV9 capsid protein and the LGETTRP (SEQ ID NO: 15) or LALGETTRP (SEQ ID NO: 16) insertion occurs immediately after at least one of the amino acid residues 451 to 461 of the AAV9 capsid and, in particular embodiments, immediately after residue 454 of the AAV9 capsid protein. In other embodiments, the capsid protein is from at least one AAV type selected from AAV1, AAV3, AAV4, AAV5 AAV6, AAV7, AAV8, AAVrh8, AAV9, AAV9e, AAVrh10, AAVrh20, AAVhu.37, AAVrh39, and AAVrh74(version 1 and version 2) (see FIG. 8), and the LGETTRP (SEQ ID NO: 15) or LALGETTRP (SEQ ID NO: 16) peptide insertion occurs immediately after an amino acid residue corresponding to at least one of the amino acid residues 451 to 461 of the AAV9 capsid or, in certain embodiments, corresponding to after the residue corresponding to residue 454 of the AAV9 capsid sequence. The alignments of these different AAV serotypes, as shown in FIG. 8, indicates corresponding amino acid residues in the different AAV capsid amino acid sequences.

In some embodiments, the retinal cell-homing peptide is not inserted into an AAV2 capsid protein, but instead the capsid protein used is from at least one AAV type selected from AAV1, AAV3, AAV4, AAV5, AAV6, AAV7, AAV8, AAVrh8, AAV9, AAV9e, AAVrh10, AAVrh20, AAVhu37, AAVrh39, and AAVrh74. In some embodiments, the retinal cell-homing peptide is not inserted between amino acids 587-588 of the AAV2 capsid protein (SEQ ID NO: 111). In some embodiments, the retinal cell-homing peptide is not inserted between amino acid residues of a different AAV serotype corresponding to amino acids 587-588 of the AAV2 capsid protein. Recombinant AAV vectors comprising one or more retinal cell-homing peptides, e.g., inserted into a surface-exposed loop of an AAV capsid coat, are referred to herein as “rAAV retinal cell-homing vectors.”

5.2.9 Additional AAV Capsid Insertion Sites

The follow summarizes insertion sites for the peptides described herein, including the peptides in Tables 1A and 1B and the dynein peptides in Table 2 immediately after amino acid residues of AAV capsids as set forth below (see also, FIG. 8):
AAV1: 138; 262-272; 450-459; 595-593; and in a particular embodiment, between 453-454 (SEQ ID NO. 110).
AAV2: 138; 262-272; 449-458; 584-592; and in particular embodiment, between 452-453 (SEQ ID NO. 111).
AAV3: 138; 262-272; 449-459; 585-593; and in particular embodiment, between 452-453 (SEQ ID NO. 112).
AAV4: 137; 256-262; 443-453; 583-591; and in particular embodiment, between 446-447 (SEQ ID NO. 113).
AAV5: 137; 252-262; 442-445; 574-582; and in particular embodiment, between 445-446 (SEQ ID NO. 114).
AAV6: 138; 262-272; 450-459; 585-593; and in particular embodiment, between 452-453 (SEQ ID NO. 115).
AAV7: 138; 263-273; 451-461; 586-594; and in particular embodiment, between 453-454 (SEQ ID NO. 116).
AAV8: 138; 263-274; 451-461; 587-595; and in particular embodiment, between 453-454 (SEQ ID NO. 117).
AAV9: 138; 262-273; 452-461; 585-593; and in particular embodiment, between 454-455 (SEQ ID NO. 118).
AAV9e: 138; 262-273; 452-461; 585-593; and in particular embodiment, between 454-455 (SEQ ID NO. 119).
AAVrh10: 138; 263-274; 452-461; 587-595; and in particular embodiment, between 454-455 (SEQ ID NO. 120).
AAVrh20: 138; 263-274; 452-461; 587-595; and in particular embodiment, between 454-455 (SEQ ID NO. 121).
AAVrh39: 138; 263-274; 452-461; 587-595; and in particular embodiment, between 454-455 (SEQ ID NO. 124).
AAVrh74: 138; 263-274; 452-461; 587-595; and in particular embodiment, between 454-455 (SEQ ID NO. 123 or SEQ ID NO: 154).
AAVhu.37: 138; 263-274; 452-461; 587-595; and in particular embodiment, between 454-455 (SEQ ID NO. 122)

In particular embodiments, the peptide insertion occurs between amino acid residues 588-589 of the AAV9 capsid, or between corresponding residues of another AAV type capsid as determined by an amino acid sequence alignment (for example, as in FIG. 8). In particular embodiments, the peptide insertion occurs immediately after amino acid residue I451 to L461, S268 and Q588 of the AAV9 capsid sequence, or immediately after corresponding residues of another AAV capsid sequence (FIG. 8).

In some embodiments, one or more peptide insertions from one or more homing domains can be used in a single system. In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

### 5.2.10 Modified Capsids

In some embodiments, AAV capsids were modified by introducing selected single to multiple amino acid substitutions which increase effective gene delivery to the CNS, detarget the liver, and/or reduce immune responses of neutralizing antibodies.

Effective gene delivery to the CNS by intravenously administered rAAV vectors requires crossing the blood brain barrier. Key clusters of residues on the AAVrh.10 capsid that enabled transport across the brain vasculature and widespread neuronal transduction in mice have recently been reported. Specifically, AAVrh.10-derived amino acids N262, G263, T264, S265, G267, S268, T269, and T273 were identified as key residues that promote crossing the BBB (Albright et al, 2018, Mapping the Structural Determinants Required for AAVrh.10 Transport across the Blood-Brain Barrier). Amino acid substitutions in capsids, such as AAV8 and AAV9 capsids that promote rAAV crossing of the blood brain barrier, transduction, detargeting of the liver and/or reduction in immune responses have been identified.

In some embodiments, provided are capsids having one or more amino acid substitutions that promote transduction and/or tissue tropism of the rAAV having the modified capsid. In particular embodiments, provided are capsids having a single mutation at amino acid 269 of the AAV8 capsid replacing alanine with serine (A269S) (see, Table 7, herein referred to as AAV8.BBB) and amino acid substitutions at corresponding positions in other AAV types. In some embodiments, provided are capsids having multiple substitutions at amino acids 263, 269, and 273 of the AAV9 capsid resulting in the following substitutions: S263G, S269T, and A273T (herein referred to as AAV9.BBB) or substitutions corresponding to these positions in other AAV types.

Exposure to the AAV capsid can generate an immune response of neutralizing antibodies. One approach to overcome this response is to map the AAV-specific neutralizing epitopes and rationally design an AAV capsid able to evade neutralization. A monoclonal antibody, specific for intact AAV9 capsids, with high neutralizing titer has recently been described (Giles et al, 2018, Mapping an Adeno-associated Virus 9-Specific Neutralizing Epitope To Develop Next-Generation Gene Delivery Vectors). The epitope was mapped to the 3-fold axis of symmetry on the capsid, specifically to residues 496-NNN-498 and 588-QAQAQT-592 (SEQ ID NO: 58). Capsid mutagenesis demonstrated that single amino acid substitution within this epitope markedly reduced binding and neutralization. In addition, in vivo studies showed that mutations in the epitope conferred a “liver-detargeting” phenotype to the mutant vectors, suggesting that the same residues are also responsible for AAV9 tropism. Liver detargeting has also been associated with substitution of amino acid 503 replacing tryptophan with arginine. Presence of the W503R mutation in the AAV9 capsid was associated with low glycan binding avidity (Shen et al, 2012, Glycan Binding Avidity Determines the Systemic Fate of Adeno-Associated Virus Type 9).

In some embodiments, provided are capsids in which the AAV8.BBB and AAV9.BBB capsids were further modified by substituting asparagines at amino acid positions 498, 499, and 500 (herein referred to as AAV8.BBB.LD) or 496, 497, and 498 (herein referred to as AAV9.BBB.LD) with alanines. In some embodiments, the AAVrh10 capsid was modified by substituting three asparagines at amino acid positions 498, 499, and 500 to alanines (AAVrh10.LD) (Table 7).

In some embodiments, provided are capsids having three asparagines at amino acid positions 496, 497, and 498 of the AAV9 capsid replaced with alanines and also tryptophan at amino acid 503 of the AAV9 capsid with arginine or capsids with substitutions corresponding to these positions in other AAV types. In some embodiments, provided are capsids having glutamine at amino acid position 474 of the AAV9 capsid substituted with alanine or capsids with substitutions corresponding to this position in other AAV types.

In some embodiments, the rAAVs described herein increase tissue-specific (such as, but not limited to, CNS) cell transduction in a subject (a human, non-human-primate, or mouse subject) or in cell culture, compared to the rAAV not comprising the peptide insertion. In some embodiments, the increase in tissue specific cell transduction is at least 2, 10, 20, 30, 40, 50, 60, 70, 80, 90, or 100 fold more than that without the peptide insertion. For example, in some embodiments, there is a 50-80 fold increase in tissue specific cell transduction compared to transduction with the same AAV type without a peptide insert. The increase in transduction may be assessed using methods described in the Examples herein and known in the art.

In some embodiments, the rAAVs described herein increase the incorporation of rAAV genomes into a cell or tissue type in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the peptide insertion. For example, in some embodiments, there is a 50-80 fold increase in genome integration compared to genome integration with the same AAV type without a peptide insert.

### 5.3. Methods of Making rAAV Molecules

Another aspect of the present invention involves making molecules disclosed herein. In some embodiments, a molecule according to the invention is made by providing a nucleotide comprising the nucleic acid sequence encoding any of the capsid protein molecules herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the AAV9 capsid protein (SEQ ID NO:118 and see FIG. 8), while retaining (or substantially retaining) biological function of the AAV9 capsid protein and the inserted peptide.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

Provided in particular embodiments are AAV9 vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV9 capsid protein (see FIG. 8), while retaining the biological function of the engineered AAV9 capsid. In certain embodiments, the encoded AAV9 capsid has the sequence of wild type AAV9, with the peptide insertion as described herein, with, in addition, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions with respect to the wild type AAV sequence and retains biological function of the AAV9 capsid. Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, or AAVrh74 vectors, with the peptide insert as described herein and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to the wild type or unengineered sequence for that AAV type and that retains its biological function.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” e Therapy 12:S18-S27, which is incorporated by reference herein in its entirety

The rAAV vector for delivering the transgene to target tissues, cells, or organs, has a tropism for that particular target tissue, cell, or organ. Tissue-specific promoters may also be used. The construct further can include expression control elements that enhance expression of the transgene driven by the vector (e.g., introns such as the chicken (3-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV9 inverted terminal repeats that flank the expression cassette; (2) control elements, which include a) the CB7 promoter, comprising the CMV enhancer/chicken (3-actin promoter, b) a chicken β-actin intron and c) a rabbit β-globin poly A signal; and (3) transgene providing (e.g., coding for) a nucleic acid or protein product of interest. In a specific embodiment, the constructs described herein comprise the following components: (1) AAV9 inverted terminal repeats that flank the expression cassette; (2) control elements, which include a) a hypoxia-inducible promoter, b) a chicken β-actin intron and c) a rabbitβ-globin poly A signal; and (3) transgene providing (e.g., coding for) a nucleic acid or protein product of interest.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl2 sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 3A-3B below provides a list of transgenes that may be used in any of the rAAV vectors described herein, in particular, in the novel insertion sites described herein, to treat or prevent the disease with which the transgene is associated, also listed in Tables 3A-3B. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 3A

| | | Possible |
|---|---|---|
| | | AAV |
| | | serotype for |
| | | delivery of |
| Disease | Transgene | transgene |
| MPS I | alpha-L-iduronidase (IDUA) | AAV9 |
| MPS II (Hunter | iduronate-2-sulfatase (IDS) | AAV9 |
| Syndrome) | | |
| ceroid lipofuscinosis | (CLN1, CLN2, CLN10, CLN13), a soluble | AAV9 |
| (Batten disease) | lysosomal protein (CLN5), a protein in the | |
| | secretory pathway (CLN11), two cytoplasmic | |
| | proteins that also peripherally associate with | |
| | membranes (CLN4, CLN14), and many | |
| | transmembrane proteins with different subcellular | |
| | locations (CLN3, CLN6, CLN7, CLN8, CLN12) | |
| MPS IIIa (Sanfilippo | heparan sulfate sulfatase (also called N- | AAV9, |
| type A Syndrome) | sulfoglucosamine sulfohydrolase (SGSH)) | Rh10 |
| MPS IIIB (Sanfilippo | N-acetyl-alpha-D-glucosaminidase (NAGLU) | AAV9 |
| type B Syndrome) | | |
| MPS VI (Maroteaux- | arylsulfatase B | AAV8 |
| Lamy Syndrome) | | |
| Morquio syndrome | Beta galactosidase or galactosamine-6-sulfatase | AAV9 |
| (MPS IV) | | |
| Gaucher disease | Glucocerebrosidase, GBA1 | AAV9 |
| (type 1, II and III) | | |
| Parkinson's Disease | Glucocerebrosidase; GBA1 | AAV9 |
| Parkinson's Disease | dopamine decarboxylase | AAV2 |
| Pompe | acid maltase; GAA | AAV9 |
| Metachromatic | Aryl sulfatase A | Rh10 |
| leukodystrophy | | |
| MPS VII (Sly | beta-glucuronidase | |
| syndrome) | | |
| MPS VIII | glucosamine-6-sulfate sulfatase | |
| MPS IX | hyaluronidase | |
| Niemann-Pick disease | sphingomyelinase | |
| Niemann-Pick disease | a npc1 gene encoding a | |
| without | cholesterol metabolizing enzyme | |
| sphingomyelinase | | |
| deficiency | | |
| Tay-Sachs disease | Alpha subunit of beta-hexosaminidase | |
| Sandhoff disease | both alpha and beta subunit of beta-hexosaminidase | |
| Fabry Disease | alpha-galactosidase | |
| Fucosidosis | Fucosidase (FUCA1 gene) | |
| Alpha-mannosidosis | alpha-mannosidase | |
| Beta-mannosidosis | Beta-mannosidase | |
| Wolman disease | cholesterol ester hydrolase | |
| Parkinson's disease | Neurturin | |
| Parkinson's disease | glial derived growth factor (GDGF) | |
| Parkinson's disease | tyrosine hydroxylase | |
| Parkinson's disease | glutamic acid decarboxylase. | |
| No disease listed | fibroblast growth factor-2 (FGF-2) | |
| No disease listed | brain derived growth factor (BDGF) | |
| No disease listed | neuraminidase deficiency with betagalactosidase | |
| (Galactosialidosis | deficiency | |
| (Goldberg syndrome)) | | |
| Spinal Muscular | SMN | AAV9 |
| Atrophy (SMA) | | |
| Friedreich's ataxia | Frataxin | AAV9 |
| | | PHP.B |
| Amyotrophic lateral | SOD1 | Rh10 |
| sclerosis (ALS) | | |
| Glycogen Storage | Glucose-6-phosphatase | AAV8 |
| Disease 1a | | |
| XLMTM | MTM1 | AAV8 or |
| | | AAV9 |
| Crigler Najjar | UGT1A1 | AAV8 |
| CPVT | CASQ2 | AAV9 |
| Rett syndrome | MECP2 | AAV9 |
| Achromatopsia | CNGB3, CNGA3, GNAT2, PDE6C | AAV8 |
| Choroidermia | CDM | AAV8 |
| Danon Disease | LAMP2 | AAV9 |

TABLE 3B

| | | Possible |
|---|---|---|
| | | AAV |
| | | serotype for |
| | | delivery of |
| Disease | Transgene | transgene |
| Cystic Fibrosis | CFTR | AAV2 |
| Duchenne Muscular Dystrophy | Mini-Dystrophin Gene | AAV2 |
| Limb Girdle Muscular Dystrophy Type | human-alpha-sarcoglycan | AAV1 |
| 2C\|Gamma-sarcoglycanopathy | | |
| Advanced Heart Failure | SERCA2a | AAV6 |
| Rheumatoid Arthritis | TNFR:Fc Fusion Gene | AAV2 |
| Leber Congenital Amaurosis | GAA | AAV1 |
| Limb Girdle Muscular Dystrophy Type | gamma-sarcoglycan | AAV1 |
| 2C\|Gamma-sarcoglycanopathy | | |
| Retinitis Pigmentosa | hMERTK | AAV2 |
| Age-Related Macular Degeneration | sFLT01 | AAV2 |
| Becker Muscular Dystrophy and | huFollistatin344 | AAV1 |
| Sporadic Inclusion Body Myositis | | |
| Parkinson's Disease | GDNF | AAV2 |
| Metachromatic Leukodystrophy (MLD) | cuARSA | AAVrh.10 |
| Hepatitis C | anti-HCV shRNA | AAV8 |
| Limb Girdle Muscular Dystrophy | hSGCA | AAVrh74* |
| Type 2D | | |
| Human Immunodeficiency Virus | PG9DP | AAV1 |
| Infections; HIV Infections (HIV-1) | | |
| Acute Intermittant Porphyria | PBGD | AAV5 |
| Leber's Hereditary Optical Neuropathy | P1ND4v2 | AAV2 |
| Alpha-1 Antitrypsin Deficiency | alpha1AT | AAVrh10 |
| Pompe Disease | hGAA | AAV9 |
| X-linked Retinoschisis | RS1 | AAV8 |
| Choroideremia | hCHM | AAV2 |
| Giant Axonal Neuropathy | JeT-GAN | AAV9 |
| Duchenne Muscular Dystrophy | rmicro-Dystrophin | AAVrh74* |
| X-linked Retinoschisis | hRS1 | AAV2 |
| Squamous Cell Head and Neck Cancer; | hAQP1 | AAV2 |
| Radiation Induced Xerostomia | | |
| Hemophilia B | Factor IX | AAVrh10/ |
| | | Rh74 |
| Homozygous FH | hLDLR | AAV8 |
| Dysferlinopathies | rAAVrh74.MHCK7.DYSF.DV | AAVrh74 |
| Hemophilia B | AAV6 ZFP nuclease | AAV6 |
| MPS I | AAV6 ZFP nuclease | AAV6 |
| Rheumatoid Arthritis | NF-kB.IFN-β | AAV5 |
| Batten/CLN6 | CLN6 | AAV9 |
| Sanfilippo Disease Type A | hSGSH | AAV9 |
| Osteoarthritis | 51L-1Ra | AAV2.5 |
| Achromatopsia | CNGA3 | AAV2tYF |
| Achromatopsia | CNGB3 | AAV8 |
| Ornithine Transcarbamylase (OTC) | OTC | scAAV8 |
| Deficiency | | |
| Hemophilia A | Factor VIII | LK03/AAV3B |
| Mucopolysaccharidosis II | ZFP nuclease | AAV6 |
| Hemophilia A | ZFP nuclease | AAV6 |
| Wet AMD | anti-VEGF | AAV8 |
| X-Linked Retinitis Pigmentosa | PGR | AAV2 |
| Mucopolysaccharidosis Type VI | hARSB | AAV8 |
| Leber Hereditary Optic Neuropathy | ND4 | AAV2 |
| X-Linked Myotubular Myopathy | MTM1 | AAV8 |
| Crigler-Najjar Syndrome | UGT1A1 | AAV8 |
| Achromatopsia | CNGB3 | AAV8 |
| Retinitis Pigmentosa | hPDE6B | AAV5 |
| X-Linked Retinitis Pigmentosa | RPGR | AAV2tYF |
| Mucopolysaccharidosis Type 3 B | hNAGLU | AAV9 |
| Duchenne Muscular Dystrophy | GALGT2 | AAVrh74 |
| Arthritis, Rheumatoid; Arthritis, | TNFR:Fc Fusion Gene | AAV2 |
| Psoriatic; Ankylosing Spondylitis | | |
| Idiopathic Parkinson's Disease | Neurturin | AAV2 |
| Alzheimer's Disease | NGF | AAV2 |
| Human Immunodeficiency Virus | tgAAC09 | AAV2 |
| Infections; HIV Infections (HIV-1) | | |
| Familial Lipoprotein Lipase Deficiency | LPL | AAV1 |
| Idiopathic Parkinson's Disease | Neurturin | AAV2 |
| Alpha-1 Antitrypsin Deficiency | hAAT | AAV1 |
| Leber Congenital Amaurosis (LCA) 2 | hRPE65v2 | AAV2 |
| Batten Disease; Late Infantile | CLN2 | AAVrh.10 |
| Neuronal Lipofuscinosis | | |
| Parkinson's Disease | GAD | AAV2 |
| Sanfilippo Disease Type A/ | N-sulfoglucosamine | AAVrh.10 |
| Mucopolysaccharidosis Type IIIA | sulfohydrolase (SGSH) gene | |
| Congestive Heart Failure | SERC2a | AAV1 |
| Becker Muscular Dystrophy and | rAAV1.CMV.huFollistatin344 | AAV1 |
| Sporadic Inclusion Body Myositis | | |
| Parkinson's Disease | hAADC-2 | AAV2 |
| Choroideremia | REP1 | AAV2 |
| CEA Specific AAV-DC-CTL | CEA | AAV2 |
| Treatment in Stage IV Gastric Cancer | | |
| Gastric Cancer | MUC1-peptide-DC-CTL | |
| Leber's Hereditary Optical Neuropathy | scAAV2-P1ND4v2 | scAAV2 |
| Aromatic Amino Acid Decarboxylase | hAADC | AAV2 |
| Deficiency | | |
| Hemophilia B | Factor IX | AAVrh10 |
| Parkinson's Disease | AADC | AAV2 |
| Leber Hereditary Optic Neuropathy | Genetic: GS010\|Drug: Placebo | AAV2 |
| SMA-Spinal Muscular Atrophy\|Gene | SMN | AAV9 |
| Therapy | | |
| Hemophilia A | B-Domain Deleted Factor VIII | AAV8 |
| MPSI | IDUA | AAV9 |
| MPS II | IDS | AAV9 |
| CLN3-Related Neuronal Ceroid- | CLN3 | AAV9 |
| Lipofuscinosis (Batten) | | |
| Limb-Girdle Muscular Dystrophy, | hSGCB | rh74 |
| Type 2E | | |
| Alzheimer Disease | APOE2 | rh10 |
| Retinitis Pigmentosa | hMERKTK | AAV2 |
| Retinitis Pigmentosa | RLBP 1 | AAV8 |
| Wet AMD | Anti-VEGF antibody | AAV2.7m8 |

For example, a rAAV vector comprising a transgene encoding glial derived growth factor (GDGF) finds use treating/preventing/managing Parkinson's disease. Generally, the rAAV vector is administered systemically. For example, the rAAV vector may be provided by intravenous, intrathecal, intra-nasal, and/or intra-peritoneal administration.

In particular aspects, the rAAVs of the present invention find use in delivery to target tissues, or target cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject tan rAAV where the peptide insertion is a homing peptide. In the case of Parkinson's, for example, a rAAV vector comprising a peptide insertion that directs the rAAV to neural tissue can be used, in particular, where the peptide insertion facilitates the rAAV in crossing the blood brain barrier to the CNS. Such peptide insertions include those derived from a neural tissue-homing domains, such as the “EPO peptide” or “HAD peptide” described herein.

For example, capsid proteins comprising an EPO peptide can find use in re-targeting AAVs to the CNS, crossing the blood-brain barrier. Capsid proteins comprising an EPO peptide further can have a protective effect on CNS tissues, e.g., where the EPO insertion binds the Innate Repair Receptor, activating the IRR biological switch, and suppressing inflammation and/or initiating CNS repair. In some embodiments, rAAVs comprising an EPO peptide of the present invention find use in one of more of the following disorders: organ ischemic injury, stroke, myocardial infarction, kidney injury, renal disease, brain injury, renal ischemia, limb ischemia, autoimmune encephalomyelitis, autoimmune neuritis, multiple sclerosis, Guillain-Barre Syndrome, neuropathic pain, diabetes mellitus complications, such as diabetic retinopathy and diabetic autonomic neuropathy, and sarcoidosis.

For a disease or disorder associated with neural tissue, an rAAV vector can be used that comprises a peptide insertion from a neural tissue-homing domain, such as any described herein. Diseases/disorders associated with neural tissue include Alzheimer's disease, amyotrophic lateral sclerosis (ALS), amyotrophic lateral sclerosis (ALS), Battens disease, Batten's Juvenile NCL form, Canavan disease, chronic pain, Friedreich's ataxia, glioblastoma multiforme, Huntington's disease, Late Infantile neuronal ceroid lipofuscinosis (LINCL), lysosomal storage disorders, Leber's congenital amaurosis, multiple sclerosis, Parkinson's disease, Pompe disease, Rett syndrome, spinal cord injury, spinal muscular atrophy (SMA), stroke, and traumatic brain injury. The vector further can contain a transgene for therapeutic/prophylactic benefit to a subject suffering from, or at risk of developing, the disease or disorder (see Tables 3A-3B).

For a disease or disorder associated with bone, an rAAV vector can be used that comprises a peptide insertion from a bone-homing domain, such as described herein.

For a disease or disorder associated with the kidneys, an rAAV vector can be used that comprises a peptide insertion from a kidney-homing domain, such as described herein.

For a disease or disorder associated with muscle, an rAAV vector can be used that comprises a peptide insertion from a muscle-homing domain, such as described herein.

For a disease or disorder associated with endothelial cells, an rAAV vector can be used that comprises a peptide insertion from an endothelial cell-homing domain, such as described herein.

For a disease or disorder associated with integrin receptors or cells expressing a particular integrin receptor, an rAAV vector can be used that comprises a peptide insertion from an integrin receptor-binding domain, such as described herein.

For a disease or disorder associated with transferrin receptors or cells expressing a transferrin receptor, such as tumors highly expressing transferrin receptors, an rAAV vector can be used that comprises a peptide insertion from an transferrin receptor-binding domain, such as described herein.

For a disease or disorder associated with tumors, an rAAV vector can be used that comprises a peptide insertion from said tumor cell-targeting domain.

For a disease or disorder associated with the retina or eye, an rAAV vector can be used that comprises a peptide insertion from said retinal cell-homing domain, including an HAD peptide. The peptide insertion increases retinal tropism, directing the rAAV to target the eye or retina of the subject, crossing the blood-eye barrier. The term “retinal cell” refers to one or more of the cell types found in or near the retina, including amacrine cells, bipolar cells, horizontal cells, Muller glial cells, photoreceptor cells (e.g., rods and cones), retinal ganglion cells (e.g., midget cells, parasol cells, bistratified cells, giant retina ganglion cells, and photosensitive ganglion cells), retinal pigmented epithelium, endothelial cells of the inner limiting membrane, and the like.

Generally, where the rAAV vector comprises a peptide insertion for retinal cell-homing, the vector is administered by in vivo injection, such as injection directly into the eye. For example, the rAAV comprising a peptide insertion for increasing retinal tropism may be injected intravitreally. In some embodiments, the rAAV for increasing retinal tropism is administered by intraocular injection, e.g., through the pars plana into the vitreous body or aqueous humor of the eye. In some embodiments, the rAAV for increasing retinal tropism is administered peribulbar injection or subconjunctival injection. One advantage of rAAV vectors with peptide insertion for retinal cell-homing, is that the subject may avoid surgery, e.g., avoiding surgery to implant the therapeutic instead delivered by injection. In certain embodiments, the therapeutic is delivered by a rAAV vector described herein by intravitreal injection, to provide a therapeutically effective amount for treating a disease or disorder associated with the eye, particularly, a disease or disorder associated with the retina of the subject. In more embodiments, treatment is achieved following a single intravitreal injection, not more than two intravitreal injections, not more than three intravitreal injections, not more than four intravitreal injections, not more than five intravitreal injections, or not more than six intravitreal injections.

Diseases/disorders associated with the eye or retina are referred to as “ocular diseases.” Nonlimiting examples of ocular diseases include anterior ischemic optic neuropathy; acute macular neuroretinopathy; Bardet-Biedl syndrome; Behcet's disease; branch retinal vein occlusion; central retinal vein occlusion; choroideremia; choroidal neovascularization; chorioretinal degeneration; cone-rod dystrophy; color vision disorders (e.g., achromatopsia, protanopia, deuteranopia, and tritanopia); congenital stationary night blindness; diabetic uveitis; epiretinal membrane disorders; inherited macular degeneration; histoplasmosis; macular degeneration (e.g., acute macular degeneration, non-exudative age related macular degeneration, exudative age related macular degeneration); diabetic retinopathy; edema (e.g., macular edema, cystoid macular edema, diabetic macular edema); glaucoma; Leber congenital amaurosis; Leber's hereditary optic neuropathy; macular telangiectasia; multifocal choroiditis; non-retinopathy diabetic retinal dysfunction; ocular trauma; ocular tumors; proliferative vitreoretinopathy (PVR); retinopathy of prematurity; retinoschisis; retinitis pigmentosa; retinal arterial occlusive disease, retinal detachment, Stargardt disease (fundus flavimaculatus); sympathetic opthalmia; uveal diffusion; uveitic retinal disease; Usher syndrome; Vogt Koyanagi-Harada (VKH) syndrome; or a posterior ocular condition associated with ocular laser or photodynamic therapy.

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

In some embodiments, ongoing treatment is indicated, e.g., on a long-term basis, such as in the ongoing treatment and/or management of chronic diseases or disorders. For example, in particular embodiments, an agent of the invention is administered over a period of time, e.g., for at least 6 months, at least one year, at least two years, at least five years, at least ten years, at least fifteen years, at least twenty years, or for the rest of the lifetime of a subject in need thereof

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In particular embodiments, such as where the transgene is intended to be expressed in the CNS, the vector is administered via lumbar puncture or via cisterna magna.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

The following examples report an analysis of surface-exposed loops on the AAV9 capsid to identify candidates for capsid engineering via insertional mutagenesis. The invention is illustrated by way of examples, describing the construction of rAAV9 capsids engineered to contain 7-mer peptides designed on the basis of the human axonemal dynein heavy chain tail. Briefly, three criteria were used for selecting surface loops that might be amenable to short peptide insertions: 1) minimal side chain interactions with adjacent loops; 2) variable sequence and structure between serotypes (lack of conserved sequences); and 3) the potential for interrupting commonly targeted neutralizing antibody epitopes. A panel of peptide insertion mutants was constructed and the individual mutants were screened for viable capsid assembly, peptide surface exposure, and potency. The top candidates were then used as templates for insertion of homing peptides to test if these peptide insertion points could be used to re-target rAAV vectors to tissues of interest. Further examples, demonstrate the increased transduction and tissue tropism for certain of the modified AAV capsids described herein.

### 6.1. Example 1

### Analysis of AAV9 Capsid

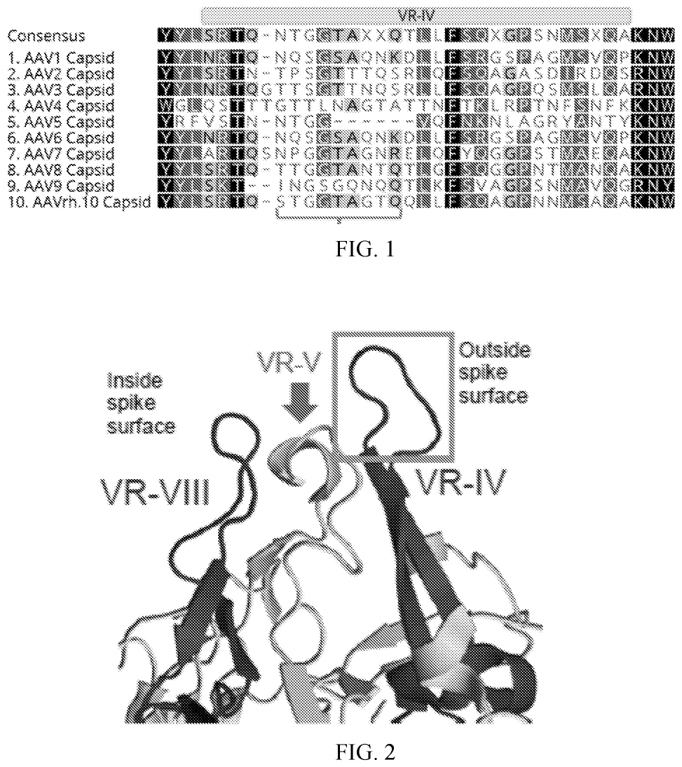

FIGS. 1 and 2 depict analysis of variable region four of the adeno-associated virus type 9 (AAV9 VR-IV) by amino acid sequence comparison to other AAVs VR-IV (FIG. 1) and protein model (FIG. 2). As seen, AAV9 VR-IV is exposed on the surface at the tip or outer surface of the 3-fold spike. Further analysis indicated that there are few side chain interactions between VR-IV and VR-V and that the sequence and structure of VR-IV is variable amongst AAV serotypes, and further that there is potential for interrupting a commonly-targeted neutralizing antibody epitope and thus, reducing immunogenicity of the modified capsid.

### 6.2. Example 2

### Construction of AAV9 Mutants

Eight AAV9 mutants were constructed, to each include a heterologous peptide but at different insertion points in the VR-IV loop. The heterologous peptide was a FLAG tag that was inserted immediately following the following residues in vectors identified as pRGNX1090-1097, as shown in Table 4.

TABLE 4

| | AAV9 VR-IV |
|---|---|
| Vector | Insertion site |
| designation | for FLAG tag |
| pRGNX1090 | I451 |
| pRGNX1091 | N452 |
| pRGNX1092 | G453 |
| pRGNX1093 | S454 |
| pRGNX1094 | G455 |
| pRGNX1095 | Q456 |
| pRGNX1096 | N457 |
| pRGNX1097 | Q458 |

### 6.3. Example 3

### Analysis of Packaging Efficiency

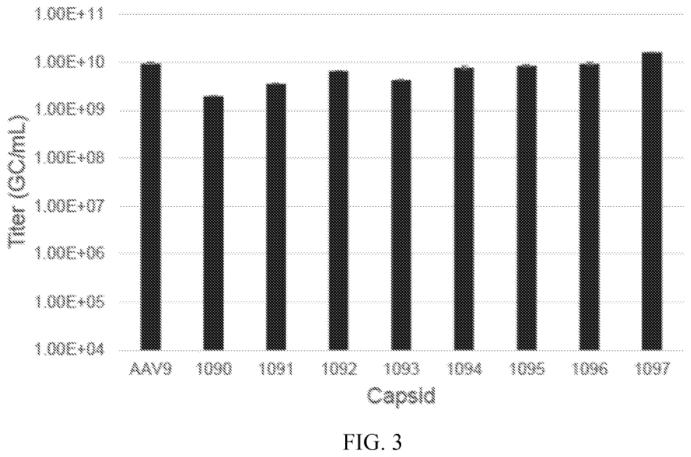

FIG. 3 depicts high packaging efficiency in terms of genome copies per mL (GC/mL) of wild type AAV9 and eight (8) candidate rAAV9 vectors (1090, 1091, 1092, 1093, 1094, 1095, 1096, and 1097), where the candidate vectors each contain a FLAG insert at different sites within AAV9′s VR-IV. All vectors were packaged with luciferase transgene in 10 mL culture to facilitate determining which insertion points did not interrupt capsid packaging; error bars represent standard error of the mean.

As seen, all candidates package with high efficiency.

### 6.4. Example 4

### Analysis of Surface FLAG Exposure

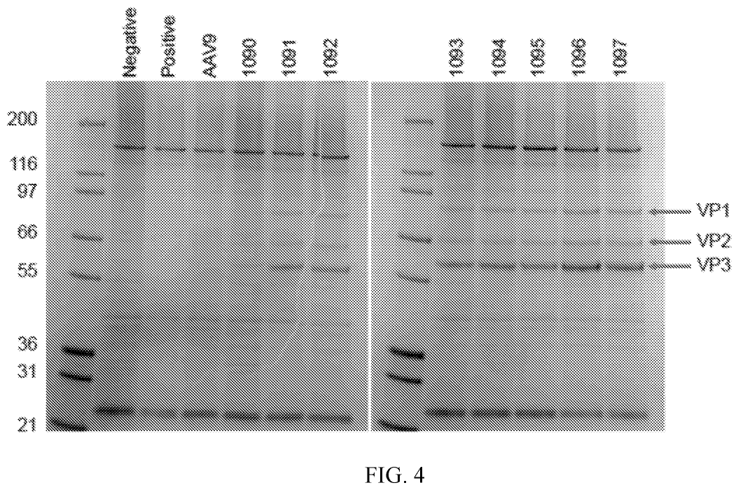

FIG. 4 depicts surface exposure of FLAG inserts in each of eight (8) candidate rAAV9 vectors (1090, 1091, 1092, 1093, 1094, 1095, 1096, and 1097), confirmed by immunoprecipitation of transduced vectors by binding to anti-FLAG resin. Binding to anti-FLAG indicates insertion points that allow formation of capsids that display the peptide insertion on the surface.

Transduced cells were lysed and centrifuged. 500 μL of cell culture supernatant was loaded on 20 μL agarose-FLAG beads and eluted with SDS-PAGE loading buffer also loaded directly on the gel. For a negative control, 293-ssc supernatant was used that contained no FLAG inserts.

As seen, 1090 had the lowest titer of the candidate vectors, indicating the least protein pulled down. Very low titers also were seen with the positive control. It is likely that not a sufficient amount of positive control had been loaded for visualization on SDS-PAGE.

### 6.5. Example 5

### Analysis of Transduction Efficiency

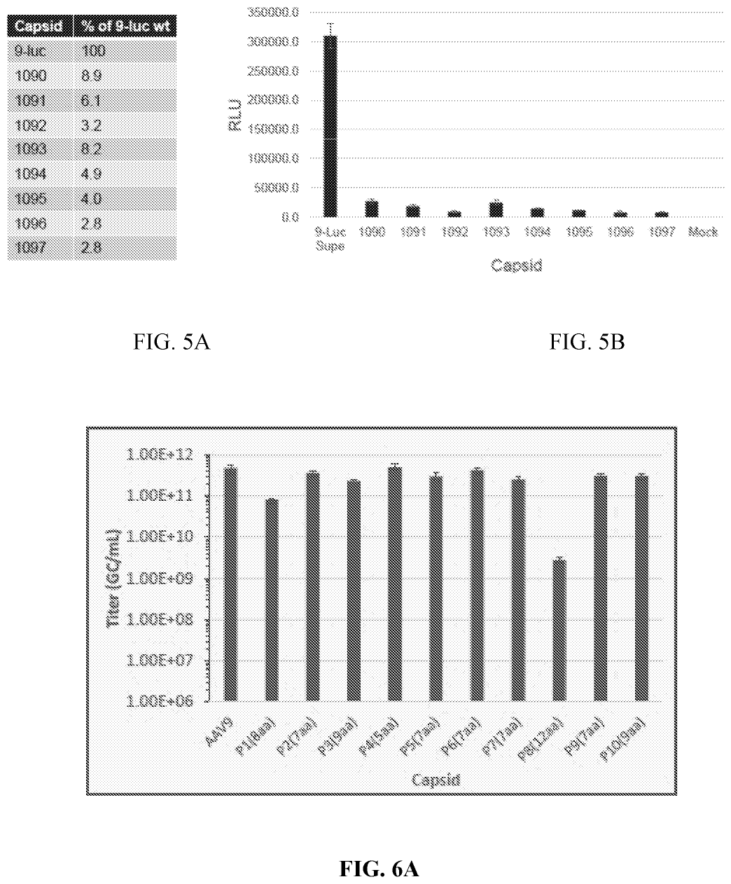

FIGS. 5A-5B depict transduction efficiency in Lec2 cells, transduced with capsid vectors carrying the luciferase gene as a transgene, that was packaged into either wild type AAV9 (9-luc), or into each of eight (8) candidate rAAV9 vectors (1090, 1091, 1092, 1093, 1094, 1095, 1096, and 1097); activity is expressed as percent luciferase activity, taking the activity of 9-luc as 100% (FIG. 5A), or as Relative Light Units (RLU) per microgram of protein (FIG. 5B).

CHO-derived Lec2 cells were grown in aMEM and 10% FBS. The Lec2 cells were transduced at a MOI of about 2×10⁸ GC vector (a MOI of about 10,000) and were treated with ViraDuctin reagent (similar results were observed on transducing Lec2 cells at a MOI of about 10,000 GC/cell but treated with 40 μg/mL zinc chloride (ZnCl₂); results not shown). Lec2 cells are proline auxotrophs from CHO.

As seen, transduction efficiency in vitro is lower than that obtained using wild type AAV9 (9-luc). Nonetheless, previous studies have shown that introduction of a homing peptide can decrease in vitro gene transfer in non-target cells (such as 293, Lec2, or HeLa), while significantly increasing in vitro gene transfer in target cells (see, e.g., Nicklin et al. 2001; and Grifman et al. 2001).

### 6.6. Example 6

### Analysis of Packaging Efficiency as a Factor of Insertion Peptide Composition and Length

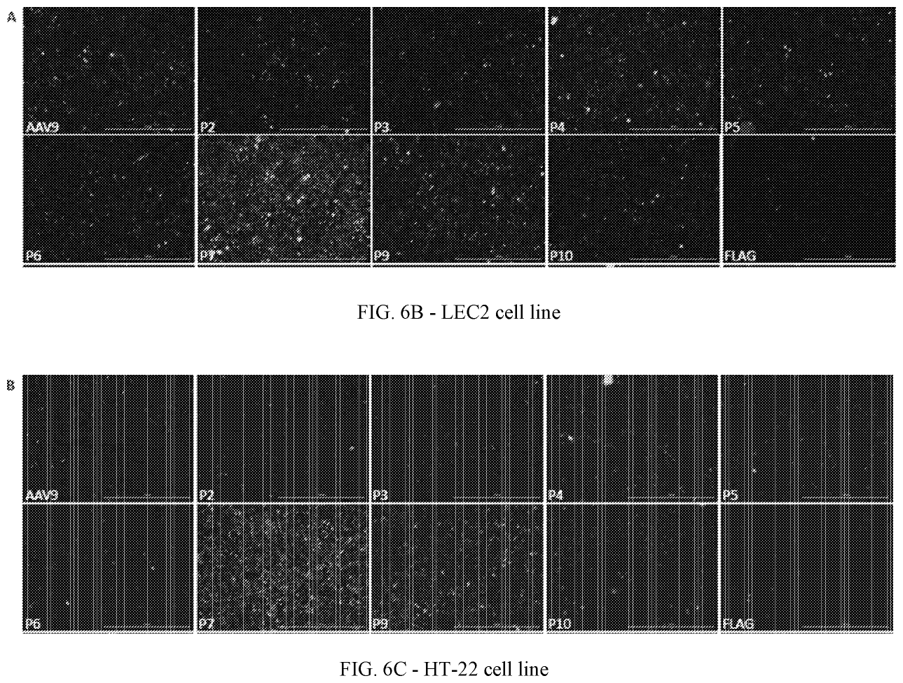

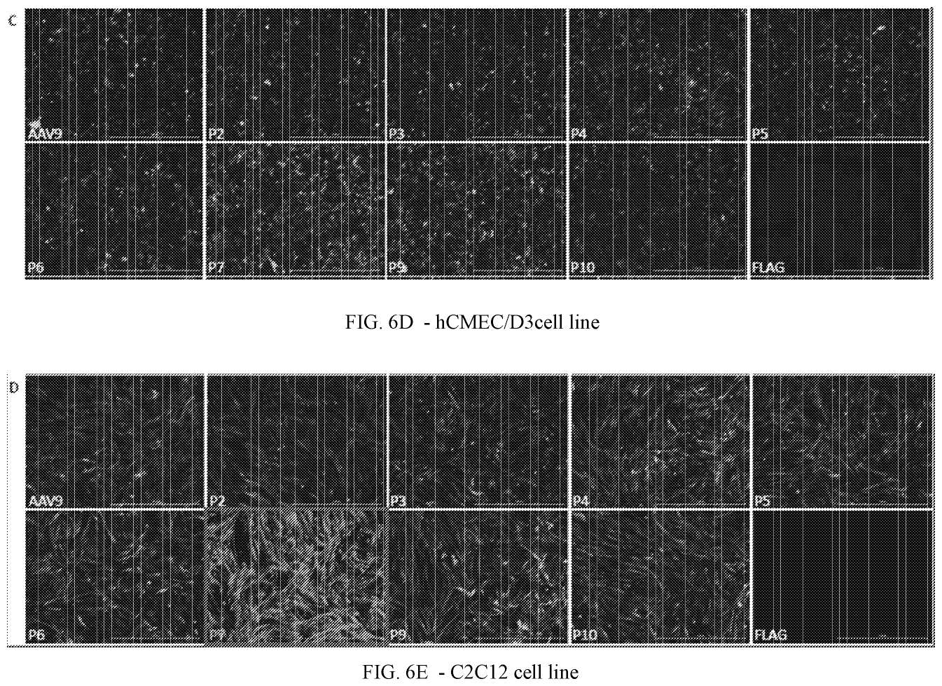

FIG. 6A depicts a bar graph illustrating that insertions immediately after S454 of AAV9 capsid (SEQ ID NO:118) of varying peptide length and composition may affect production efficiencies of AAV particles in a packaging cell line. Ten peptides of varying composition and length were inserted after S454 (between residues 454 and 455) within AAV9 VR-IV. qPCR was performed on harvested supernatant of transfected suspension HEK293 cells five days post-transfection. The results depicted in the bar graph demonstrate that the nature and length of the insertions may affect the ability of AAV particles to be produced at high titer and packaged in 293 cells. (Error bars represent standard error of the mean length of peptide, which is noted on the Y-axis in parenthesis.)

AAV9 vectors having an capsid protein containing a homing peptide of the following peptide sequences (Table 5) at the S454 insertion site were studied. Suspension-adapted HEK293 cells were seeded at 1x10⁶ cells/mL one day before transduction in 10mL of media. Triple plasmid DNA transfections were done with PElpro® (Polypus transfection) at a DNA:PEI ratio of 1:1.75. Cells were spun down and supernatant harvested five days post-transfection and stored at -80° C.

TABLE 5

| | Tissue or Target | Peptide | SEQ |
|---|---|---|---|
| Peptide# | Designation | Sequence | ID NO: |
| P1 | Bone1 (D8) | DDDDDDDD | 9 |
| P2 | Brain1 | LSSRLDA | 10 |
| P3 | Brain2 | CLSSRLDAC | 11 |
| P4 | Kidney1 | LPVAS | 13 |
| P5 | Kidney2 | CLPVASC | 12 |
| P6 | Muscle1 | ASSLNIA | 14 |
| P7 | TfR1 | HAIYPRH | 17 |
| P8 | TfR2 | THRPPMWSPVWP | 18 |
| P9 | TfR3 | RTIGPSV | 19 |
| P10 | TfR4 | CRTIGPSVC | 20 |

qPCR was performed on harvested supernatant of transfected suspension HEK293 cells five days post-transfection. Samples were subjected to DNase I treatment to remove residual plasmid or cellular DNA and then heat treated to inactivate DNase I and denature capsids. Samples were titered via qPCR using TaqMan Universal PCR Master Mix, No AmpEraseUNG (ThermoFisherScientific) and primer/probe against the polyA sequence packaged in the transgene construct. Standard curves were established using RGX-501 vector BDS.

Peptide insertions directly after S454 ranging from 5 to 10 amino acids in length produced AAV particles having adequate titer, whereas an upper size limit is possible, with significant packaging deficiencies observed for the peptide insertion having a length of 12 amino acids.

### 6.7. Example 7

### Homing Peptides Alter the Transduction Properties of AAV9 In Vitro when Inserted after S454.

FIGS. 6B-E depict fluorescence images of cell cultures of (FIG. 6B) Lec2 cell line (sialic acid-deficient epithelial cell line) (FIG. 6C) HT-22 cell line (neuronal cell line), (FIG. 6D) hCMEC/D3 cell line (brain endothelial cell line), and (FIG. 6E) C2C12 cell line (muscle cell line). AAV9 wild type and S454 insertion homing peptide capsids of Table 5 containing GFP transgene were used to transduce the noted cell lines.

Cell lines were plated at 5-20×10³ cells/well (depending on the cell line) in 96-well 24 hours before transduction. Cells were transduced with AAV9-GFP vectors (with or without insertions) at 1×10¹° particles/well and analyzed via Cytation5 (BioTek) 48-96 hours after transduction, depending on the difference in expression rate in each cell line. Lec2 cells were cultured as in Example 5, blood-brain barrier hCMEC/D3 (EMD Millipore) cells were cultured according to manufacturer's protocol, HT-22 and HU-17 cells were cultured in DMEM and 10% FBS, and C2C12 myoblasts were plated in DMEM and 10% FBS and differentiated for three days pre-transfection in DMEM supplemented with 2% horse serum and 0.1% insulin. AAV9.S454.FLAG showed low transduction levels in every cell type tested.

Images show that homing peptides can alter the transduction properties of AAV9 in vitro when inserted after S454 in the AAV9 capsid protein, as compared to unmodified AAV9 capsid. P7 (TfR1 peptide, HAIYPRH (SEQ ID NO: 17)) for all cell lines show the highest rate of transduction followed by P9 (TfR3 peptide, RTIGPSV (SEQ ID NO: 19)). P4 (Kidney1 peptide, LPVAS (SEQ ID NO: 13)) showed a slightly higher rate of transduction than that of AAV9 wildtype for all cell types. Higher transduction rates were observed for P6 (Muscle1 peptide, ASSLNIA (SEQ ID NO: 14)) in the brain endothelial hCMEC/D3 cell line and the C2C12 muscle cell line cultures as compared to the Lec2 and HT-22 cell line cultures. P1 vector was not included in images due to extremely low transduction efficiency, and P8 vector was not included due to low titer.

### 6.8. Example 8

### Analysis of Human Axonemal Dynein (HAD)

FIGS. 7A-7M depict the amino acid sequences for heavy chain tail domains of human axonemal dynein 1-12, 14 and 17, respectively.

### 6.9. Example 9

### Analysis of AAV Capsids for Peptide Insertion Points

FIG. 8 depicts alignment of AAVs 1-9e, rh10, rh20, rh39, rh74 and hu.37 capsid sequences within insertion sites for human axonemal dynein peptides within or near the initiation codon of VP2, variable region 1 (VR-I), variable region 4 (VR-IV), and variable region 8 (VR-VIII) highlighted in grey; a particular insertion site within variable region eight (VR-VIII) of each capsid protein is shown by the symbol “#” (after amino acid residue 588 according to the amino acid numbering of AAV9).

### 6.10. Example 10

### Construction of rAAV Capsid containing ARA290

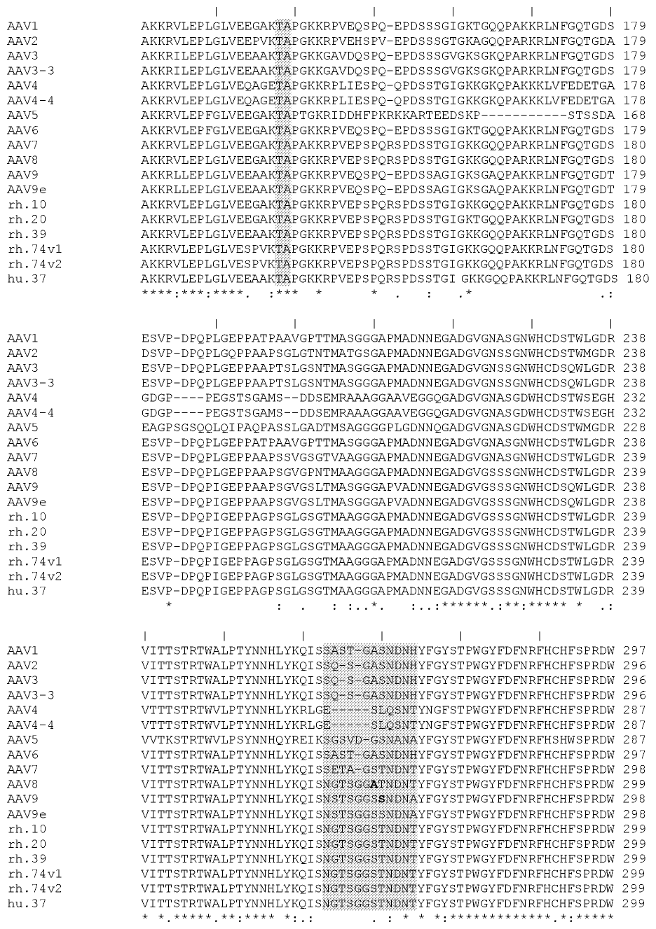

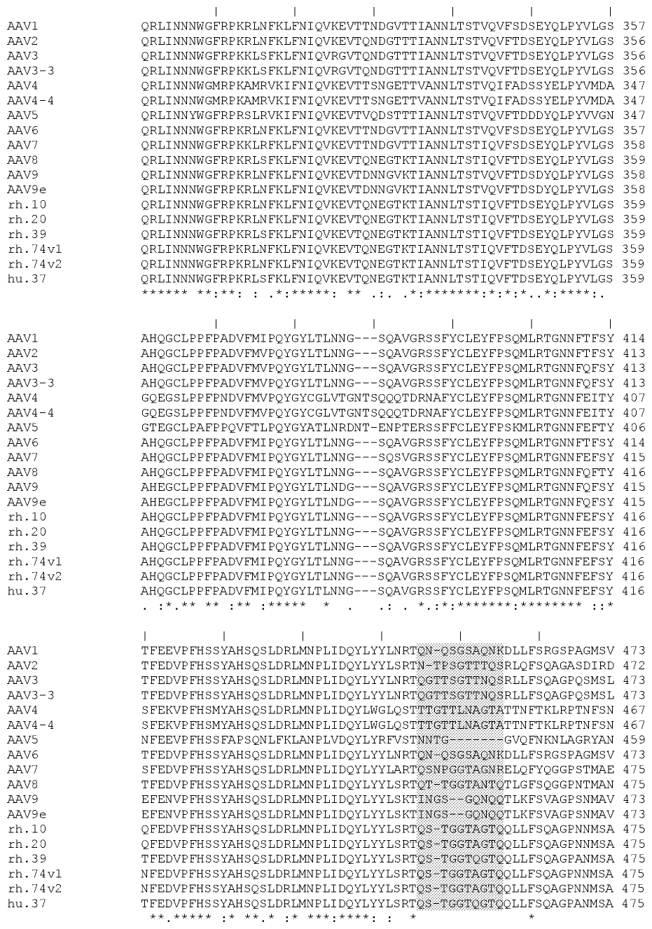

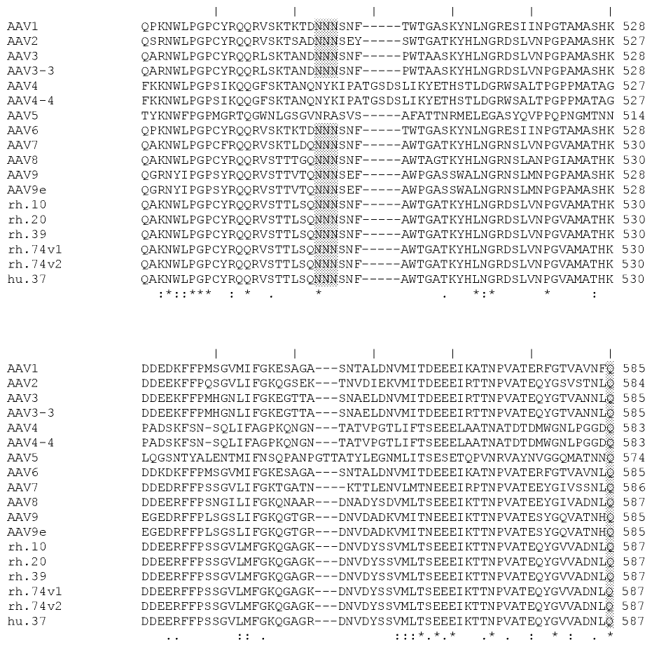

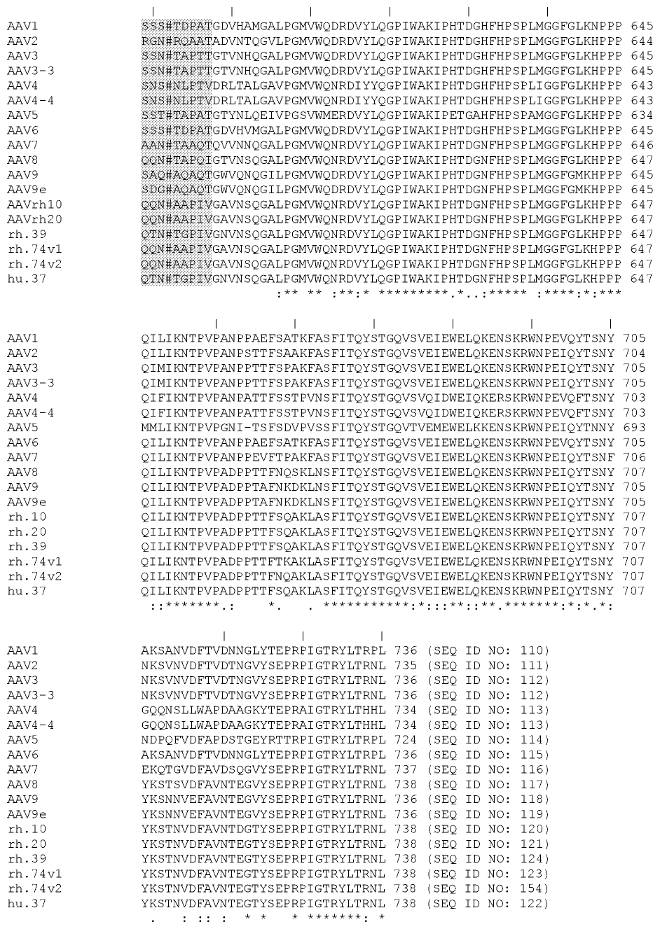

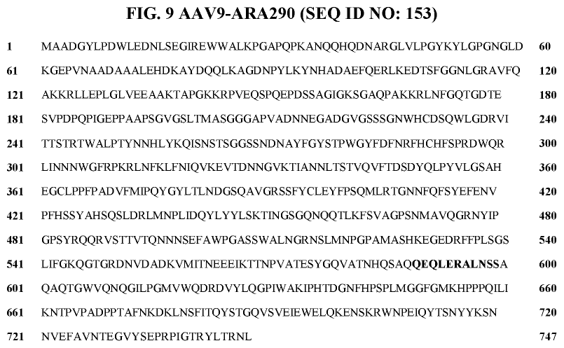

FIG. 9 depicts the amino acid sequence for a recombinant AAV9 vector capsid including a peptide insertion of ARA290 between Q588 and A589 of the AAV9 capsid amino acid sequence (SEQ ID NO: 153).

### 6.11. Example 11

### Comparison of AAV Genome Copies/μg genomic DNA of Various Vectors

FIG. 10 depicts copies of GFP (green fluorescent protein) transgene expressed in mouse brain cells, following administration of the AAV vectors: AAV9; AAV.PHP.eB; AAV.hDyn (AAV9 with TLAAPFK (SEQ ID NO: 2) between 588-589 with no other amino acid modifications to the capsid sequence); AAV.PHP.S; and AAV.PHP.SH (see Table 10).

AAV.PHP.B is a capsid having a TLAVPFK (SEQ ID NO: 27) insertion in AAV9 capsid, with no other amino acid modifications to the capsid sequence. AAV.PHP.eB is a capsid having a TLAVPFK (SEQ ID NO: 27) insertion in AAV9 capsid, with two amino acid modifications of the capsid sequence upstream of the PHP.B insertion (see also Table 10). Table 6A summarizes the capsids utilized in the study.

TABLE 6A

| | | | | | SEQ |
|---|---|---|---|---|---|
| | Parent | | Location of | Peptide | ID |
| Name | capsid | Mutation | insertion 2 | 2 | NO: |
| AAV9 | AAV9 | — | — | — | |
| PHP.B | AAV9 | — | 588_589 | TLAVPFK | 27 |
| PHP.eB | AAV9 | 586A_587Q | 588_589 | TLAVPFK | 27 |
| | | delinsDG | | | |
| AAV.hDyn | AAV9 | — | 588_589 | TLAAPFK | 2 |
| AAV.PHP.S | AAV9 | — | 588_589 | QAVRTSL | 23 |
| AAV.PHP.SH | AAV9 | — | 588_589 | QAVRTSH | 24 |

### Materials and Methods

Constructs of AAV9, AAV.PHPeB, AAV.hDyn, AAV.PHP.S and AAV.PHP.SH encoding GFP transgene were prepared and formulated in 1× PBS+0.001% Pluronic. Female C57BL/6 mice were randomized into treatment groups base on Day 1 bodyweight. Five groups of female C57BL/6 mice were each intravenously administered AAV9.GFP, AAV.PHPeB.GFP, AAV.hDyn.GFP, AAV.PHP.S.GFP or AAV.PHP.SH.GFP in accordance with Table 6B, below. The dosing volume was 10 mL/kg (0.200 mL/20 g mouse). The mice were 8-12 weeks of age at the start date. At day 15 post administration, the animals were euthanized, and peripheral tissues were collected, including brain tissue, liver, forelimb biceps, heart, kidney, lung, ovaries, and the sciatic nerve.

TABLE 6B

| | | | Formulation | | |
|---|---|---|---|---|---|
| Gr. | N | Agent | dose | Route | Schedule |
| 1 | 9 | AAV9 | 2.5E12 GC/kg | iv | day 1 |
| 2 | 5 | PHPeB | 2.5E12 GC/kg | iv | day 1 |
| 3 | 5 | hDyn | 2.5E12 GC/kg | iv | day 1 |
| 4 | 5 | PHP.S | 2.5E12 GC/kg | iv | day 1 |
| 5 | 5 | PHP.SH | 2.5E12 GC/kg | iv | day 1 |

Quantitiative PCR (qPCR) was used to determine the number of vector genomes per μg of brain genomic DNA. Brain samples from injected mice were processed and genomic DNA was isolated using Blood and Tissue Genomic DNA kit from Qiagen. The qPCR assay was run on a QuantStudio 5 instrument (Life Technologies Inc) using primer-probe combination specific for eGFP following a standard curve method.

The AAV vector genome copies per μg of brain genomic DNA was at least a log higher in mice that were administered AAV.hDyn compared to all other AAV serotypes: AAV9, AAV.PHPeB, PHP.S, and PHP.SH (see FIG. 10). As seen in this study, GC/μg genomic DNA is highest for AAV.hDyn, which is AAV9 capsid containing the “TLAAPFK” (SEQ ID NO: 2) peptide insert (a peptide from human axonemal dynein) between residues 588-589 of the AAV9 capsid. The study demonstrated transduction in mouse brain at greater than 1E04 GC/μg transgene on average in 5 mice systemically administered AAV.hDyn carrying eGFP. Other modified AAV9 capsids, however, including the vector AAV.PHPeB, which contains the “TLAVPFK” (SEQ ID NO: 27) sequence (a peptide from mouse dynein) demonstrated transduction in mouse brain at less than 1E03 GC/μg transgene upon systemic treatment.

### 6.12. Example 12

### Use of Tissue-Homing rAAV Vector in Methods of Treatment

A disorder is identified that can be treated/prevented by providing a nucleic acid (transgene) (see Tables 3A-3B). A subject having the disorder associated with a target tissue is identified. The subject is administered a first amount of a rAAV vector of the invention, where the vector comprises a capsid protein with a peptide insertion that homes to the target tissue and carries the transgene to be delivered. If needed, the subject is administered a second or third dose of the vector, until a therapeutically effective amount of the transgene is delivered to the target tissue to provide a therapeutic or prophylactic benefit to the subject.

In some embodiments, methods are provided for administering a transgene to the retina, whereby an AAV.hDyn capsid encapsidating the transgene is administered intravenously, systemically or intravitreally.

### 6.13. Example 13

### Construction of rAAV Capsid containing TLAAPFK (SEQ ID NO: 2)

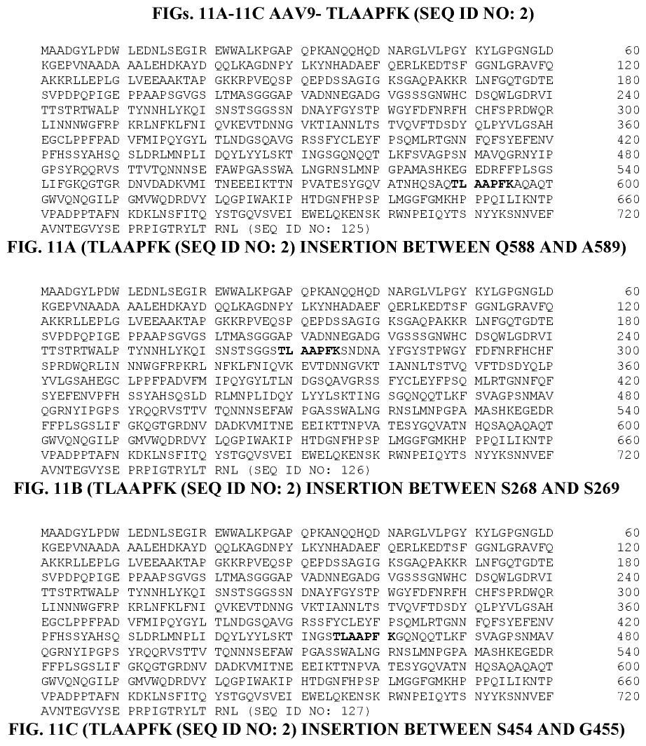

FIG. 11A depicts the amino acid sequence for a recombinant AAV9 vector capsid including a peptide insertion of amino acid sequence TLAAPFK (SEQ ID NO: 2) between Q588 and A589 of VR-IIIV. Inserted peptide in bold.

FIG. 11B depicts the amino acid sequence for a recombinant AAV9 vector capsid including a peptide insertion of amino acid sequence TLAAPFK (SEQ ID NO: 2) between S268 and S269 of VR-III. Inserted peptide in bold.

FIG. 11C depicts the amino acid sequence for a recombinant AAV9 vector capsid including a peptide insertion of amino acid sequence TLAAPFK (SEQ ID NO: 2) between S454 and G455 of VR-IV. Inserted peptide in bold.

### 6.14. Example 14

### Construction of rAAV Capsid containing KMQVPFQ (SEQ ID NO: 1)

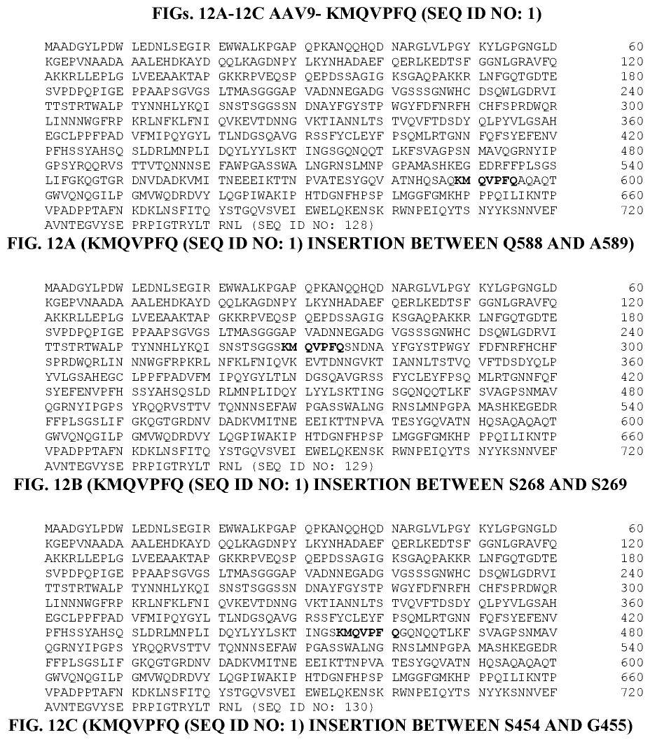

FIG. 12A depicts the amino acid sequence for a recombinant AAV9 vector capsid including a peptide insertion of amino acid sequence KMQVPFQ (SEQ ID NO: 1) between Q588 and A589 of VR-VIII. Inserted peptide in bold.

FIG. 12B depicts the amino acid sequence for a recombinant AAV9 vector capsid including a peptide insertion of amino acid sequence KMQVPFQ (SEQ ID NO: 1) between S268 and S269 of VR-III. Inserted peptide in bold.

FIG. 12C depicts the amino acid sequence for a recombinant AAV9 vector capsid including a peptide insertion of amino acid sequence KMQVPFQ (SEQ ID NO: 1) between S454 and G455 of VR-IV. Inserted peptide in bold.

### 6.15. Example 15

### Construction of rAAV Capsid containing QQAAPSF (SEQ ID NO: 3)

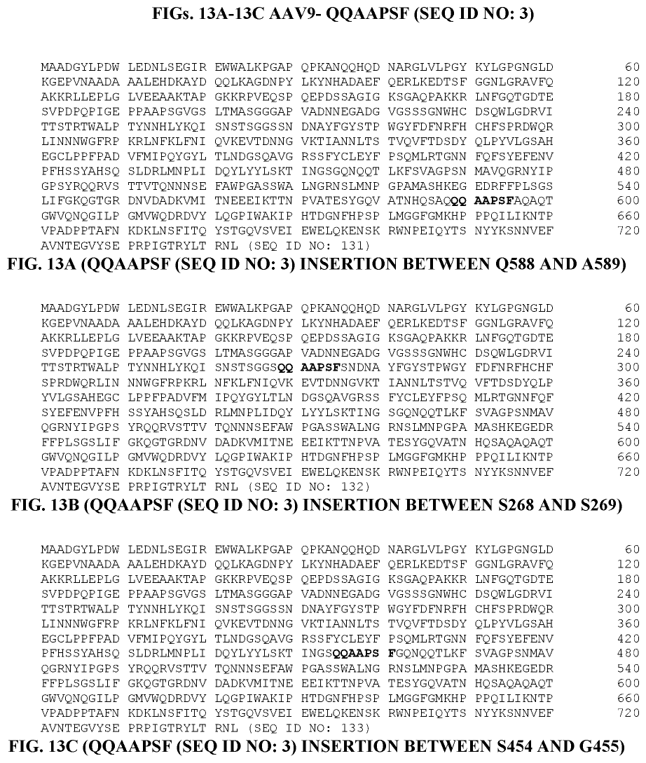

FIG. 13A depicts the amino acid sequence for a recombinant AAV9 vector capsid including a peptide insertion of amino acid sequence QQAAPSF (SEQ ID NO: 3) between Q588 and A589 of VR-VIII. Inserted peptide in bold.

FIG. 13B depicts the amino acid sequence for a recombinant AAV9 vector capsid including a peptide insertion of amino acid sequence QQAAPSF (SEQ ID NO: 3) between S268 and S269 of VR-III. Inserted peptide in bold.

FIG. 13C depicts the amino acid sequence for a recombinant AAV9 vector capsid including a peptide insertion of amino acid sequence QQAAPSF (SEQ ID NO: 3) between S454 and G455 of VR-IV. Inserted peptide in bold.

### 6.16. Example 16

### Construction of rAAV Capsid containing RYNAPFK (SEQ ID NO: 4)

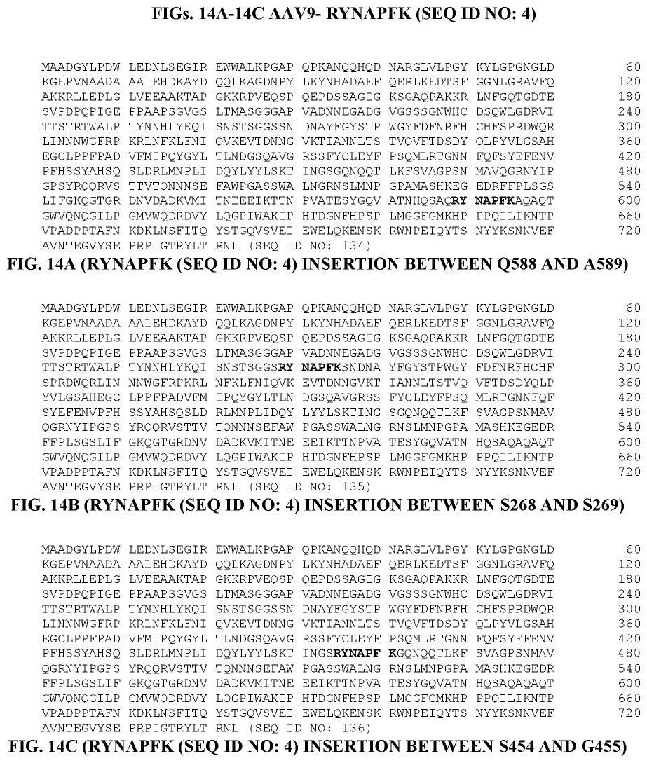

FIG. 14A depicts the amino acid sequence for a recombinant AAV9 vector capsid including a peptide insertion of amino acid sequence RYNAPFK (SEQ ID NO: 4) between Q588 and A589 of VR-VIII. Inserted peptide in bold.

FIG. 14B depicts the amino acid sequence for a recombinant AAV9 vector capsid including a peptide insertion of amino acid sequence RYNAPFK (SEQ ID NO: 4) between S268 and S269 of VR-III. Inserted peptide in bold.

FIG. 14C depicts the amino acid sequence for a recombinant AAV9 vector capsid including a peptide insertion of amino acid sequence RYNAPFK (SEQ ID NO: 4) between S454 and G455 of VR-IV. Inserted peptide in bold.

### 6.17. Example 17

### Construction of rAAV Capsid containing LKLPPIV (SEQ ID NO: 5)

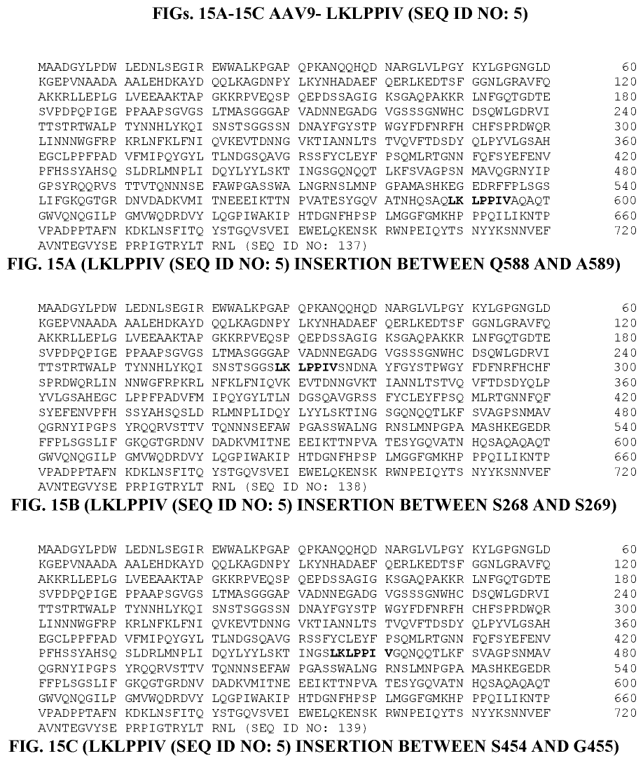

FIG. 15A depicts the amino acid sequence for a recombinant AAV9 vector capsid including a peptide insertion of amino acid sequence LKLPPIV (SEQ ID NO: 5) between Q588 and A589 of VR-VIII. Inserted peptide in bold.

FIG. 15B depicts the amino acid sequence for a recombinant AAV9 vector capsid including a peptide insertion of amino acid sequence LKLPPIV (SEQ ID NO: 5) between S268 and S269 of VR-III. Inserted peptide in bold.

FIG. 15C depicts the amino acid sequence for a recombinant AAV9 vector capsid including a peptide insertion of amino acid sequence LKLPPIV (SEQ ID NO: 5) between S454 and G455 of VR-IV. Inserted peptide in bold.

### 6.18. Example 18

### Construction of rAAV Capsid containing PFIKPFE (SEQ ID NO: 6)

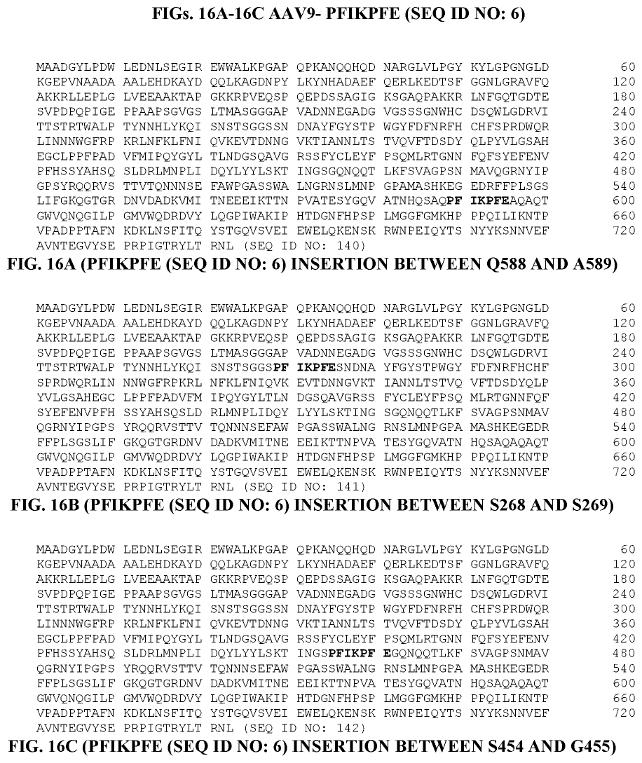

FIG. 16A depicts the amino acid sequence for a recombinant AAV9 vector capsid including a peptide insertion of amino acid sequence PFIKPFE (SEQ ID NO: 6) between Q588 and A589 of VR-VIII. Inserted peptide in bold.

FIG. 16B depicts the amino acid sequence for a recombinant AAV9 vector capsid including a peptide insertion of amino acid sequence PFIKPFE (SEQ ID NO: 6) between S268 and S269 of VR-III. Inserted peptide in bold.

FIG. 16C depicts the amino acid sequence for a recombinant AAV9 vector capsid including a peptide insertion of amino acid sequence PFIKPFE (SEQ ID NO: 6) between S454 and G455 of VR-IV. Inserted peptide in bold.

### 6.19. Example 19

### Construction of rAAV Capsid containing TLSLPWK (SEQ ID NO: 7)

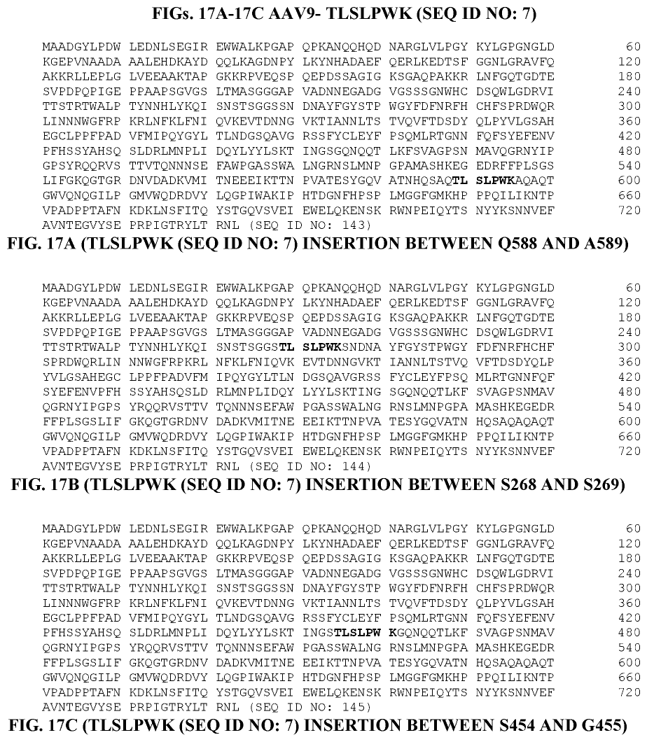

FIG. 17A depicts the amino acid sequence for a recombinant AAV9 vector capsid including a peptide insertion of amino acid sequence TLSLPWK (SEQ ID NO: 7) between Q588 and A589 of VR-VIII. Inserted peptide in bold.

FIG. 17B depicts the amino acid sequence for a recombinant AAV9 vector capsid including a peptide insertion of amino acid sequence TLSLPWK (SEQ ID NO: 7) between S268 and S269 of VR-III. Inserted peptide in bold.

FIG. 17C depicts the amino acid sequence for a recombinant AAV9 vector capsid including a peptide insertion of amino acid sequence TLSLPWK (SEQ ID NO: 7) between S454 and G455 of VR-IV. Inserted peptide in bold.

### 6.20. Example 20

### Construction of rAAV Capsid containing LGETTRP (SEQ ID NO: 15)

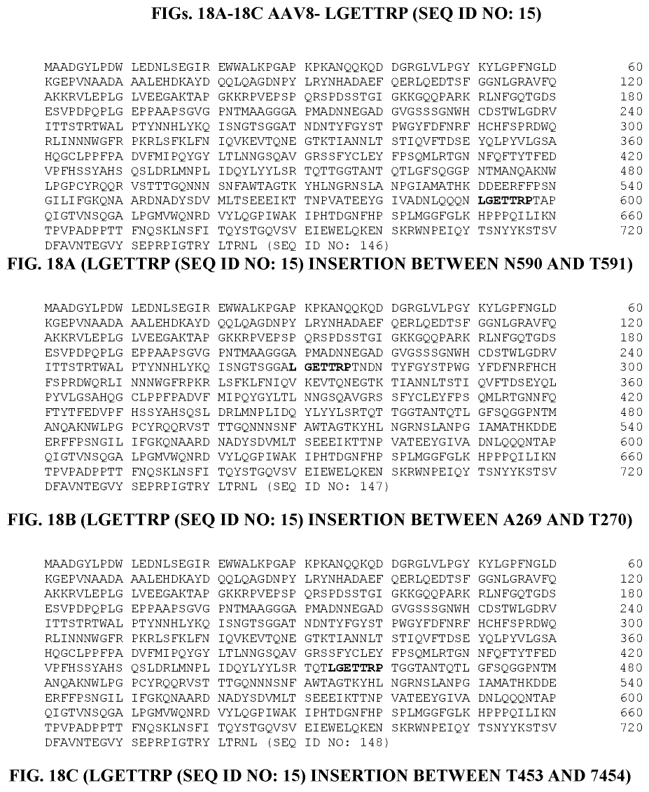

FIG. 18A depicts the amino acid sequence for a recombinant AAV8 vector capsid including a peptide insertion of amino acid sequence LGETTRP (SEQ ID NO: 15) between N590 and T591 of VR-VIII. Inserted peptide in bold.

FIG. 18B depicts the amino acid sequence for a recombinant AAV8 vector capsid including a peptide insertion of amino acid sequence LGETTRP (SEQ ID NO: 15) between A269 and T270 of VR-III. Inserted peptide in bold.

FIG. 18C depicts the amino acid sequence for a recombinant AAV8 vector capsid including a peptide insertion of amino acid sequence LGETTRP (SEQ ID NO: 15) between T453 and T454 of VR-IV. Inserted peptide in bold.

### 6.21. Example 21

### Construction of rAAV Capsid containing LALGETTRP (SEQ ID NO: 16)

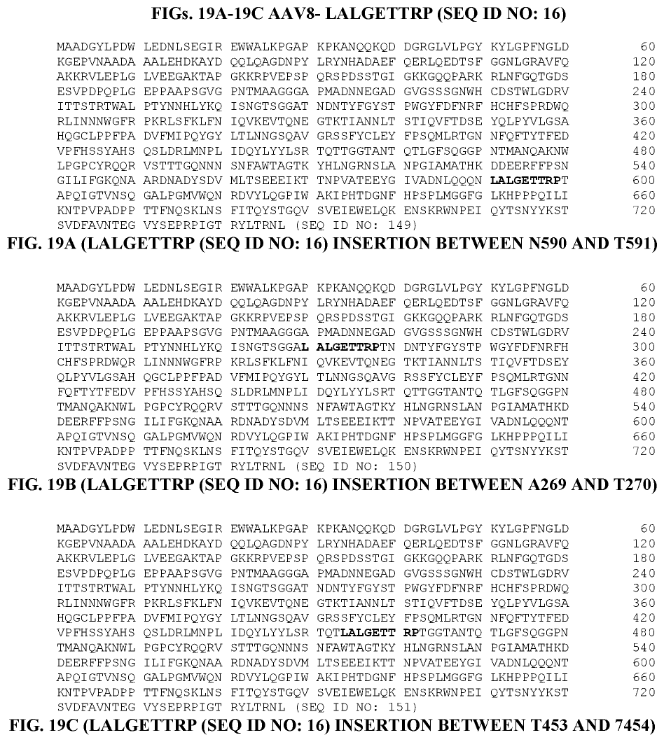

FIG. 19A depicts the amino acid sequence for a recombinant AAV8 vector capsid including a peptide insertion of amino acid sequence LALGETTRP (SEQ ID NO: 16) between N590 and T591 of VR-VIII. Inserted peptide in bold.

FIG. 19B depicts the amino acid sequence for a recombinant AAV8 vector capsid including a peptide insertion of amino acid sequence LALGETTRP (SEQ ID NO: 16) between A269 and T270 of VR-III. Inserted peptide in bold.

FIG. 19C depicts the amino acid sequence for a recombinant AAV8 vector capsid including a peptide insertion of amino acid sequence LALGETTRP (SEQ ID NO: 16) between T453 and T454 of VR-IV. Inserted peptide in bold.

### 6.22. Example 22

### Assessment of Modified Capsids In Vitro and In Vivo

AAV capsid sequences were modified either by peptide insertions or guided mutagenesis and pooled to give a bar-coded library packaged with a GFP expression cassette. The modified vectors were then evaluated in an in vitro assay, as well as for in vivo bio-distribution in mice using next generation sequencing (NGS) and quantitative PCR. AAV.hDyn was identified as a high brain transduction vector from this pool and was further evaluated in individual delivery studies in mice to characterize its transduction profile. Additionally, immunohistochemistry analysis of brain sections was performed to understand the cellular tropism of this vector.

### 6.22.1 Example 22A

### In Vitro Testing of Transduction an Crossing Blood Brain Barrier

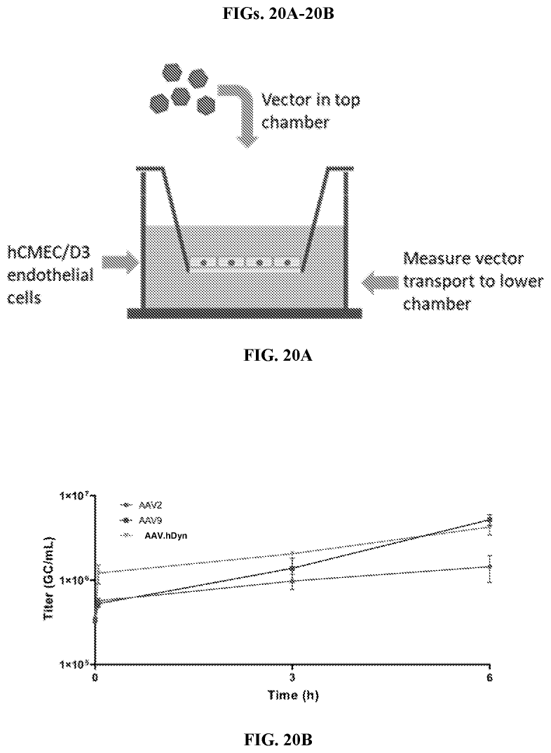

The ability of the modified capsids to cross the blood brain barrier was tested in an in vitro transwell assay using hCMEC/D3 BBB cells (SCC066, Millipore-Sigma) (see FIGS. 20A-20B). More specifically, the assay was essentially adapted from Sade, H. et al. (2014 PLoS ONE 9(4): e96340) A human Blood-Brain Barrier transcytosis assay reveals Antibody Transcytosis influenced by pH-dependent Receptor Binding, April 2014, Vol. 9, Issue 4; and Zhang, X., Blood-brain barrier shuttle peptides enhance AAV transduction in the brain after systemic administration, 2018 Biomaterials 176: 71-83. Briefly, 5×10⁴ hCMEC/D3 cells/cm² were seeded in collagen-coated transwell inserts in a 12-well plate. Each insert contained 500 μL media and the lower chamber contained 1 mL media. Media was replaced every second day. The supernatant was removed at 10 days post-seeding (the zero (0) timepoint). At this 0 timepoint, the cells were transduced by adding 1×10⁹ GC of vector to the upper insert chamber media. 10 μL lower chamber supernatant samples were removed for testing at intervals 0.5, 3, 6, and 23 hours post-transduction. Each condition (vector) was tested in duplicate, and measured for titer via qPCR against PolyA in triplicate.

FIGS. 20A-20B depict an in vitro transwell assay for AAV.hDyn (AAV9 with TLAAPFK (SEQ ID NO: 2) between amino acid residues 588-589) crossing a blood brain barrier (BBB) cell layer (FIG. 20A), and results showing that AAV.hDyn (indicated by inverted triangles in the figure) crosses the BBB cell layer of the assay faster than AAV9 (squares), as well as faster and to a greater extent than AAV2 (circles) (FIG. 20B). The developed in vitro assay predicted enhanced BBB cross-trafficking and similar assays can be used to predict targeting to other organs as well.

### 6.22.2 Example 22B

### Transduction and Biodistribution of Modified Capsids

### 6.22.2.1 Materials and Methods

Capsid modifications were performed on widely used AAV capsids including AAV8, AAV9, and AAVrh.10 by inserting various peptide sequences after the position S454 of the VR-IV (Table 7) or after position Q588 of the VR-VIII surface exposed loop of the AAV capsid, as well as insertions after the initiation codon of VP2, which begins at amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74, and hu.37) (FIG. 8) (see also Table 10 for certain capsid sequences). Selected single to multiple amino acid mutations were also used for modifying the capsids. See also, Yost et al., Structure-guided engineering of surface exposed loops on AAV Capsids. 2019. ASGCT Annual Meeting; and Wu et al., 2000 J. Virology (supra). It was confirmed that packaging efficiency was not negatively impacted following any of these capsid modifications in small scale.

rAAVs with certain modified capsids were tested for transduction in vitro in Lec2 cells as described above in Example 5. Modified AAVs tested for transduction in Lec2 cells as follows: eB 588 Ad, eB 588 Hep, eB 588 p79, eB 588 Rab, AAV9 588 Ad, AAV9 588 Hep, AAV9 588 p79, AAV9 588 Rab, eB VP2 Ad, eB VP2 Hep, eB VP2 p79, eB VP2 Rab, AAV9 VP2 Ad, AAV9 VP2 Hep, AAV9 VP2 p79, AAV9 VP2 Rab as compared to AAV9. See Table 7B below for identity of AAV capsids.

To test biodistribution, modified AAVs were packaged with an eGFP transgene cassette containing specific barcodes corresponding to each individual capsid. Novel barcoded vectors were pooled and injected into mice in order to increase the efficiency of screening.

To analyse the bio-distribution of genetically altered AAV vectors, various vectors encoding GFP were prepared and formulated in 1× PBS+0.0001% Pluronic acid. All vectors were made with cis plasmids containing a ten (10) bp barcode to enable next-generation sequencing (NGS) library (pool) preparation. Three (3) vector pools (Study 1, Study 2 and Study 3 vectors) were injected intravenously into a cohort of 5 female C57Bl/6 mice in accordance with Tables 7A-C. The dosing volume was 10 mL/kg (0.2 mL/20 g mouse) for each.

The mice were randomized into treatment groups based on Day 1 bodyweight and their age at start date was 8-12 weeks. At day 15 post administration, the animals were euthanized and peripheral tissues were collected, including brain, kidney, liver, sciatic nerve, lung, heart, and muscle tissue. In the studies where selected capsids from the pool were injected individually, the same protocol was followed

Genomic DNA (gDNA) was isolated from tissue samples using DNeasy Blood and Tissue kit (69506) from Qiagen. Each vector's barcode region was amplified with primers containing overlaps for NGS and unique dual indexing (UDI) and multiplex sequencing strategies, as recommended by the manufacturer (Illumina). Illumina MiSeq using reagent nano and micro kits v2 (MS-103-1001/1002) were used to determine the relative abundance of each barcoded AAV vector per sample collected from the mice. Accordingly, each vector sample in Tables 7A-C below was barcoded as noted above to allow for each read to be identified and sorted before the final data analysis. The data was normalized based on the composition of AAVs in the originally injected pool and quantified using the total genome copy number obtained from qPCR analysis with a primer-probe combination specific to the barcoded sample.

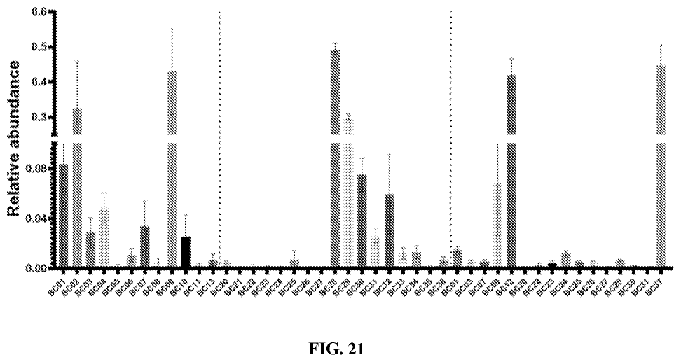

TABLE 7A

| | | | Insertion | | |
|---|---|---|---|---|---|
| Study 1 | Name | Capsid | Point | Peptide | Notes |
| BC01 | AAV9 | AAV9 | — | — | Blue bar, FIG. 21 |
| BCO2 | PHP.eB | PHP.eB | 588_589 | TLAVPFK | |
| | | | | (SEQ ID | |
| | | | | NO: 27) | |
| BC03 | AAV8.BBB | Modified | — | — | A269S |
| | | AAV8 | | | |
| BC04 | AAV9.BBB | Modified | — | — | S263G/S269T/A273T |
| | | AAV9 | | | |
| BC05 | AAV8.BBB.LD | Modified | — | — | A2695, 498- |
| | | AAV8 | | | NNN/AAA-500 |
| BC06 | AAV9.BBB.LD | Modified | — | — | 5263G/5269T/A273T, |
| | | AAV9 | | | 496-NNN/AAA-498 |
| BC07 | rh.10 | rh.10 | — | — | |
| BC08 | rh.10.LD | Modified | - | — | 498-NNN/AAA-500 |
| | | rh.10 | | | |
| BC09 | AAV.hDyn | modifiedAAV9 | 588_589 | TLAAPFK | Orange bar, FIG. |
| | | | | (SEQ ID | 21 |
| | | | | NO: 2) | |
| BC10 | PHP.S | PHP.S | 588_589 | QAVRTSL | — |
| | | | | (SEQ ID | |
| | | | | NO: 23) | |
| BC11 | PHP.SH | PHP.SH | 588_589 | QAVRTSH | — |
| | | | | (SEQ ID | |
| | | | | NO: 24) | |
| BC13 | rh39 | rh.39 | — | — | |

TABLE 7B

| | | | Insertion | | |
|---|---|---|---|---|---|
| Study 2 | Name | Capsid | Point | Peptide | Notes |
| BC20 | eB 588 Ad | PHP.eB | 588_589 | SITLVKSTQTV | Replaces |
| | | | | (SEQ ID NO: 21) | TLAVPFK peptide |
| | | | | | (SEQ ID NO: 27) |
| BC21 | eB 588 Hep | PHP.eB | 588_589 | TILSRSTQTG (SEQ | Replaces |
| | | | | ID NO: 22) | TLAVPFK peptide |
| | | | | | (SEQ ID NO: 27) |
| BC22 | eB 588 p79 | PHP.eB | 588_589 | VVMVGEKPITITQ | Replaces |
| | | | | HSVETEG (SEQ ID | TLAVPFK peptide |
| | | | | NO: 25) | (SEQ ID NO: 27) |
| BC23 | eB 588 Rab | PHP.eB | 588_589 | RSSEEDKSTQTT | Replaces |
| | | | | (SEQ ID NO: 26) | TLAVPFK peptide |
| | | | | | (SEQ ID NO: 27) |
| BC24 | 9 588 Ad | AAV9 | 588_589 | SITLVKSTQTV | |
| | | | | (SEQ ID NO: 21) | |
| BC25 | 9 588 Hep | AAV9 | 588_589 | TILSRSTQTG (SEQ | |
| | | | | ID NO: 22) | |
| BC26 | 9 588 p79 | AAV9 | 588_589 | VVMVGEKPITITQ | |
| | | | | HSVETEG (SEQ ID | |
| | | | | NO: 25) | |
| BC27 | 9 588 Rab | AAV9 | 588_589 | RSSEEDKSTQTT | |
| | | | | (SEQ ID NO: 26) | |
| BC28 | eB VP2 Ad | PHP.eB | 138_139 | SITLVKSTQTV | Also has |
| | | | | (SEQ ID NO: 21) | TLAVPFK (SEQ |
| | | | | | ID NO: 27) insert |
| | | | | | after residue 588 |
| BC29 | eB VP2 Hep | PHP.eB | 138_139 | TILSRSTQTG (SEQ | Also has |
| | | | | ID NO: 22) | TLAVPFK (SEQ |
| | | | | | ID NO: 27) insert |
| | | | | | after residue 588 |
| BC30 | eB VP2 p79 | PHP.eB | 138_139 | VVMVGEKPITITQ | Also has |
| | | | | HSVETEG (SEQ ID | TLAVPFK (SEQ |
| | | | | NO: 25) | ID NO: 27) insert |
| | | | | | after residue 588 |
| BC31 | AAV9 | AAV9 | — | — | |
| BC32 | eB VP2 Rab | PHP.eB | 138_139 | RSSEEDKSTQTT | Also has |
| | | | | (SEQ ID NO: 26) | TLAVPFK (SEQ |
| | | | | | ID NO: 27) insert |
| | | | | | after residue 588 |
| BC33 | 9 VP2 Ad | AAV9 | 138_139 | SITLVKSTQTV | |
| | | | | (SEQ ID NO: 21) | |
| BC34 | 9 VP2 Hep | AAV9 | 138_139 | TILSRSTQTG (SEQ | |
| | | | | ID NO: 22) | |
| BC35 | 9 VP2 p79 | AAV9 | 138_139 | VVMVGEKPITITQ | |
| | | | | HSVETEG (SEQ ID | |
| | | | | NO: 25) | |
| BC36 | 9 VP2 Rab | AAV9 | 138_139 | RSSEEDKSTQTT | |
| | | | | (SEQ ID NO: 26) | |

TABLE 7C

| | | | Insertion | | |
|---|---|---|---|---|---|
| Study 3 | Name | Capsid | Point | Peptide | Notes |
| BC01 | AAV9 | AAV9 | — | — | |
| BC03 | AAV8-BBB | AAV8 | — | — | A269S |
| BC07 | rh10 | rh.10 | — | — | |
| BC09 | AAV.hDyn | AAV.hDyn | 588_589 | TLAAPFK | |
| | | | | (SEQ ID NO: 2) | |
| BC12 | PHP.B | PHP.B | 588_589 | TLAVPFK | |
| | | | | (SEQ ID NO: | |
| | | | | 27) | |
| BC20 | AAV9 S454- | AAV9 | 454_455 | DDDDDDDD | |
| | D8 | | | (SEQ ID NO: 9) | |
| BC22 | AAV9 S454- | AAV9 | 454_455 | LSSRLDA | |
| | Brain1 | | | (SEQ ID NO: | |
| | | | | 10) | |
| BC23 | AAV9 S454- | AAV9 | 454_455 | CLSSRLDAC | |
| | Brian1C | | | (SEQ ID NO: | |
| | | | | 11) | |
| BC24 | AAV9 S454- | AAV9 | 454_455 | LPVAS (SEQ | |
| | Kidney1 | | | ID NO: 13) | |
| BC25 | AAV9 S454- | AAV9 | 454_455 | CLPVASC | |
| | Kidney1C | | | (SEQ ID NO: | |
| | | | | 12) | |
| BC26 | AAV9 S454- | AAV9 | 454_455 | ASSLNIA | |
| | Muscle1 | | | (SEQ ID NO: | |
| | | | | 14) | |
| BC27 | AAV9 S454- | AAV9 | 454_455 | HAIYPRH | |
| | TfR1 | | | (SEQ ID NO: | |
| | | | | 17) | |
| BC29 | AAV9 S454- | AAV9 | 454_455 | RTIGPSV | |
| | TfR3 | | | (SEQ ID NO: | |
| | | | | 19) | |
| BC30 | AAV9 S454- | AAV9 | 454_455 | CRTIGPSVC | |
| | TfR4 | | | (SEQ ID NO: | |
| | | | | 20) | |
| BC31 | AAV9 S454- | AAV9 | 454_455 | DYKDDDDK | |
| | FLAG | | | (SEQ ID NO: | |
| | | | | 52) | |
| BC37 | pRGX1005- | PHP.eB | 588_589 | TLAVPFK | |
| | PHP.eB (no | | | (SEQ ID NO: | |
| | BC) | | | 27) | |

In the studies where selected capsids from the pool were injected individually, qPCR was used to determine the number of vector genomes per μg of tissue genomic DNA. qPCR was done on a QuantStudio 5 (Life Technologies, Inc.) using primer-probe combination specific for eGFP following a standard curve method (FIG. 22).

From the study where individual vectors were injected into mice for characterization, formal in fixed mouse brains were sectioned at 40 μm thickness on a vibrating blade microtome (VT1000S, Leica) and the floating sections were probed with antibodies against GFP to look at the cellular distribution of the delivered vectors.

More specifically, fixed brains from the mice injected with AAV.hDyn were sectioned using a Vibratome (Leica, VT-1000) and the GFP expression was evaluated using an anti-GFP antibody (AB3080, Millipore Sigma), Vectastain ABC kit (PK-6100, Vector Labs) and DAB Peroxidase kit (SK-4100, Vector Labs). Broad distribution of GFP expressing cells were present throughout the brain in mice injected with AAV.hDyn, including distribution in the cortex, striatum, and hippocampus of the brain. FIGS. 23A-23C show the images from these regions and the scale bar is 400um (discussed below).

### 6.22.2.2 Results

Results are shown in FIG. 21, FIGS. 22A-22H, and FIGS. 23A-23C.

Data for the Lec2 cell transduction assay not shown. The AAV9 588 Hep (AAV9 with the peptide TILSRSTQTG (SEQ ID NO: 22) (DLC-AS2 in Table 1b) inserted after position 588) exhibited significantly greater transduction (4-fold) than wild type AAV9, and AAV9 VP2 Ad (AAV9 with the peptide SITLVKSTQTV (SEQ ID NO: 21) (DLC-AS1 in Table 1b) inserted after position 138), AAV9 VP2 Hep (AAV9 with the peptide TILSRSTQTG (SEQ ID NO: 22) (DLC-AS2 in Table 1b) inserted after position 138), and AAV9 VP2 Rab (AAV9 with the peptide RSSEEDKSTQTT (SEQ ID NO: 26) (DLC-AS4 in Table 1b) inserted after position 138) exhibited slightly greater transduction of the Lec2 cells relative to AAV9. The other AAVs assayed exhibited lower levels of transduction than AAV9.

FIG. 21 depicts results of Next Generation Sequencing (NGS) analysis of brain gDNA, revealing relative abundances (percent composition) of the capsid pool delivered to mouse brains following intravenous injection. The data was normalized based on the composition of AAVs in the originally injected pool and quantified using the total genome copy number obtained from qPCR analysis with a primer-probe combination specific to the eGFP sequence. Data shown are from three different experiments. Dotted lines indicate which vectors were pooled together. Parental AAV9 was used as standard and included in each pool. The “BC” identifiers are as indicated in Tables 7A, 7B and 7C above.

FIGS. 22A-22H depict an in vivo transduction profile of AAV.hDyn in female C57Bl/6 mice, showing copy number/microgram gDNA in naive mice, or mice injected with either AAV9 or AAV.hDyn in brain (FIG. 22A), liver (FIG. 22B), heart (FIG. 22C), lung (FIG. 22D), kidney (FIG. 22E), skeletal muscle (FIG. 22F), sciatic nerve (FIG. 22G), and ovary (FIG. 22H), where AAV.hDyn shows increased brain bio-distribution compared to AAV9. The AAV vector genome copies per μg of brain genomic DNA was at least a log higher in mice that were administered AAV.hDyn compared to the parental AAV9 vector.

FIGS. 23A-23C show images from the regions analysed in the Immunohistochemical Analysis described above; scale bar is 400 μm. FIGS. 23A-23C depict distribution of GFP from AAV.hDyn throughout the brain, where images of immunohistochemical staining of brain sections from the striatum (FIG. 23A), hippocampus (FIG. 23B), and cortex (FIG. 23C) revealed a global transduction of the brain by the modified vector.

### 6.22.2.3 Conclusions

AAV capsid modifications performed either by insertions in surface exposed loops of VR-IV and VR-VIII or by specific amino acid mutations did not affect their packaging efficiency and were able to produce similar titers in the production system described herein.

Intravenous administration of AAV.hDyn to mice resulted in higher relative abundance of the viral genome and greater brain cell transduction than other modified AAV vectors and AAV9 tested.

### 6.23. Example 23

### Homing Peptide Kidney1C Depicts Enhanced Transduction Following Systemic Delivery

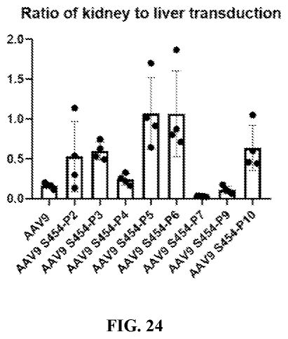

AAV capsid sequences were modified either by peptide insertions and pooled to give a bar-coded library packaged with a GFP expression cassette. The bio-distribution profile of the modified AAV9 vectors were then evaluated in vivo in mice using next generation sequencing (NGS) and quantitative PCR. Recombinant AAV9 vectors including peptide insertion of amino acid sequences CLPVASC (SEQ ID NO: 12) (Kidney1C) or ASSLNIA (SEQ ID NO: 14) (Muscle 1) between S454 and G455 of VR-IV showed increased transduction efficiency of the kidney compared to the liver (FIG. 24).

### 6.23.1 Materials and Methods

Capsid modifications were performed on AAV9 by inserting various homing peptide sequences after the position S454 of the VR-IV surface exposed loop of the AAV capsid. It was confirmed that packaging efficiency was not negatively impacted following any of these capsid modifications in small scale. Peptide sequences are shown in Table 8 below. All modified AAVs were packaged with an eGFP transgene cassette containing specific barcodes corresponding to each individual capsid. These novel barcoded vectors were pooled in order to increase the efficiency of screening (as explained above in Example 22B; see Study 3, Table 7C).

Genetically altered AAV vectors were injected intravenously into mice as explained above in Example 22B with respect to Study 3 altered vectors. The data was normalized based on the composition of AAVs in the originally injected pool and quantified using the total genome copy number obtained from qPCR analysis with a primer-probe combination specific to the eGFP sequence, and kidney to liver tissue targeting was more closely examined.

### 6.23.2 Results and Conclusions

FIG. 24 depicts the ratio of kidney to liver in vivo transduction of AAV9 S454 vectors with different homing peptide insertions (Table 8) in female C57B1/6 mice. Kidney-to-liver transduction versus the total kidney transduction of the pool for modified capsids was used for the calculation. AAV9 S454 Kidney1 and AAV9 S454 Kidney2 (Kidney 1C) show increased kidney bio-distribution compared to parental AAV9. While the parental AAV9 vector shows increased transduction of the liver compared to the kidney with a ratio of ˜0.25, insertion of the kidney homing peptide 1C (and also Muscle 1) results in an increase of this ratio to ˜1.0. The AAV vector genome copies per μg of kidney gDNA was at least a 5-fold higher in mice that were administered AAV9 S454 Kidney1 or AAV9 S454 Musclel compared to all other AAV9 S454 vectors (see FIG. 24).

TABLE 8
Homing peptides used in biodistribution study

| | | Location | | | SEQ |
|---|---|---|---|---|---|
| | | of Peptide | | Peptide | ID |
| Name | Capsid | Insertion | Peptide Name | Sequence | NO: |
| AAV9 | AAV9 | 454_455 | — | — | |
| AAV9 S454-P2 | AAV9 | 454_455 | Brain1 | LSSRLDA | 10 |
| AAV9 S454-P3 | AAV9 | 454_455 | Brain2 | CLSSRLDAC | 11 |
| | | | (Brain1C) | | |
| AAV9 S454-P4 | AAV9 | 454_455 | Kidney1 | LPVAS | 13 |
| AAV9 S454-P5 | AAV9 | 454_455 | Kidney2 | CLPVASC | 12 |
| | | | (Kidney 1C) | | |
| AAV9 S454-P6 | AAV9 | 454_455 | Muscle1 | ASSLNIA | 14 |
| AAV9 S454-P7 | AAV9 | 454_455 | Tfr1 | HAIYPRH | 17 |
| AAV9 S454-P9 | AAV9 | 454_455 | Tfr3 | RTIGPSV | 19 |
| AAV9 S454- | AAV9 | 454_455 | Tfr4 | CRTIGPSVC | 20 |
| P10 | | | | | |

AAV capsid modifications performed by insertions of different homing peptides in surface exposed loop VR-IV did not affect their packaging efficiency and were able to produce similar titers in the production system described herein.

Intravenous administration of AAV9 S454 Kidney1 and AAV9 S454 Kidney1C to mice resulted in higher relative abundance of the viral genome and greater kidney cell transduction than other modified AAV9 vectors and the parental AAV9 vector tested. Intravenous administration of the AAV9 S454 Kidney1 or AAV9 S454 Musclel vector to mice resulted also in lower liver cell transduction.

### 6.24. Example 24

### Construction of rAAV Capsid containing TLAVPFK (SEQ ID NO: 27)

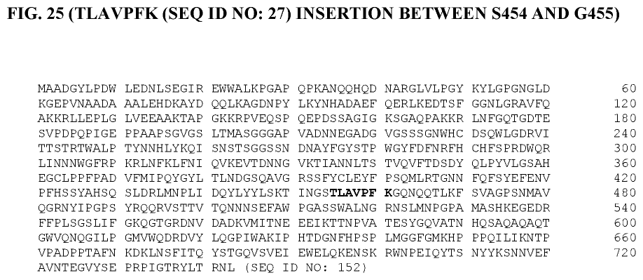

FIG. 25 depicts the amino acid sequence for a recombinant AAV9 vector capsid including a peptide insertion of amino acid sequence TLAVPFK (SEQ ID NO: 27) between S454 and G455 of VR-IV.

### 6.25. Example 25

### Biodistribution of an rAAV Vector Pool in Cynomolgus Monkeys

The administration, in vivo and post-mortem observations, and biodistribution of a pool of recombinant AAVs having engineered capsids and a GFP transgene will be evaluated following a single intravenous, intracerebroventricular or intravitreal injection in cynomolgus monkeys (Table 9). The pool contains multiple capsids each of which contains a unique barcode identification allowing identification using next generation sequencing (NGS) analysis following administration to cynomolgus monkeys. The cynomolgus monkey is chosen as the test system because of its established usefulness and acceptance as a model for AAV biodistribution studies in a large animal species and for further translation to human. All animals on this study are naïve with respect to prior treatment. The pool may comprise at least the following recombinant AAVs having the engineered capsids listed in Table 9.

TABLE 9
Recombinant AAVs for Cynomolgus monkey study

| | | | | | Peptide |
|---|---|---|---|---|---|
| | | Capsid | Location of | | SEQ ID |
| Name | Capsid | modification | insertion | Peptide | NO: |
| AAV8 | AAV8 | — | — | — | |
| AAV8.BBB | Modified | A269S | — | — | |
| | AAV8 | | | | |
| AAV8.BBB.LD | Modified | A269S, 498- | — | — | |
| | AAV8 | NNN/AAA-500 | | | |
| AAV9 | AAV9 | — | — | — | |
| AAV9 S454- | AAV9 | — | 454_455 | LSSRLDA | 10 |
| Brain1 | | | | | |
| AAV9 S454- | AAV9 | — | 454_455 | CLSSRLDAC | 11 |
| Brain1C | | | | | |
| AAV9 S454-D8 | AAV9 | — | 454_455 | DDDDDDDD | 9 |
| AAV9 S454- | AAV9 | — | 454_455 | LPVAS | 13 |
| Kidney1 | | | | | |
| AAV9 S454- | AAV9 | — | 454_455 | CLPVASC | 12 |
| Kidney1C | | | | | |
| AAV9 S454- | AAV9 | — | 454_455 | ASSLNIA | 14 |
| Muscle1 | | | | | |
| AAV9 S454-Tfr1 | AAV9 | — | 454_455 | HAIYPRH | 17 |
| AAV9 S454-Tfr3 | AAV9 | — | 454_455 | RTIGPSV | 19 |
| AAV9 S454- | AAV9 | — | 454_455 | CRTIGPSVC | 20 |
| TfR3C | | | | | |
| AAV9.496NNN/ | Modified | 498-NNN/AAA- | — | — | |
| AAA498 | AAV9 | 500 | | | |
| AAV9.496NNN/ | Modified | 498-NNN/AAA- | — | — | |
| AAA498.W503R | AAV9 | 500, W503R | | | |
| AAV9.588Ad | AAV9 | — | 588_589 | SITLVKSTQ | 21 |
| | | | | TV | |
| AAV9.588Herp | AAV9 | — | 588_589 | TILSRSTQT G | 22 |
| AAV9.BBB | Modified | S263G/S269T/ | — | — | |
| | AAV9 | A273T | | | |
| AAV9.BBB.LD | Modified | S263G/S269T/ | — | — | |
| | AAV9 | A273T, 496- | | | |
| | | NNN/AAA-498 | | | |
| AAV9.Q474A | Modified | Q474A | — | — | |
| | AAV9 | | | | |
| AAV9.W503R | Modified | W503R | — | — | |
| | AAV9 | | | | |
| AAVPHPeB.VP | PHP.eB | — | 138_139 | SITLVKSTQ | 21 |
| 2Ad | | | | TV | |
| AAVPHPeB.VP | PHP.eB | — | 138_139 | TILSRSTQT | 22 |
| 2Herp | | | | G | |
| PHP.B | AAV9 | — | 588_589 | TLAVPFK | 27 |
| PHP.eB | Modified | A587D, Q588G | 588_589 | TLAVPFK | 27 |
| | PHP.B | | | | |
| PHP.hB | AAV9 | — | 588_589 | QAVRTSL | 23 |
| PHP.S | | | | | |
| PHP.SH | AAV9 | — | 588_589 | QAVRTSH | 24 |

### 6.25.1. Study Design

Nine female cynomolgus animals will be used. Animals judged suitable for experimentation based on clinical sign data and prescreening antibody titers will be placed in study groups by body weight using computer-generated random numbers. Three different routes of administration will be used and relevant tissues collected to evaluate the biodistribution (measured by NGS and PCR) associated with the different routes. Three animals will be implanted with a catheter in the left lateral ventricle for intracerebroventricular (ICV) dose administration (Group 1), three animals will receive a single intravenous infusion (Group 2) and three animals will receive a single intravitreal injection (Group 3). Two animals will serve as replacement animals and will be implanted if required. Animals in Group 1 will have an MRI scan to determine coordinates for proper ICV catheter placement.

The IV infusion will be administered at a rate of 3 mL/min followed by 0.2 mL of vehicle to flush the dose from the IV catheter. The three intravenous animals will receive a single dose of the pooled recombinant AAVs at a volume of 4 mL/kg. The total dose (vg) and dose volume (mL/kg) will be recorded in the raw data. Based on literature review and previous studies in non-human primates, the IV dose of 1×10¹³ GC/kg body weight was determined to be required to have the desired distribution in the CNS from a systemic delivery as well as the peripheral tissues including skeletal muscle.

The ICV implanted animals will receive a single bolus dose at a volume of 1 mL of AAV-NAV-GFPbc (by slow infusion, approximately 0.1 mL/min) followed by 0.1 mL of vehicle to flush the dose from the catheter system. The ICV dose is based on distribution data from a previous non-human primate study to support current clinical programs.

The intravitreal (IVT) injection will be administered bilateral as a bolus injection at a dose volume of 50 μL.

### 6.25.2. Observations and Examinations

Clinical signs will be recorded at least once daily beginning approximately two weeks prior to initiation of dosing and continuing throughout the study period. The animals will be observed for signs of clinical effects, illness, and/or death. Additional observations may be recorded based upon the condition of the animal at the discretion of the Study Director and/or technicians.

Ophthalmological examinations will be performed on Group 3 animals prior to dose administration, and on Days 2, 8, 15 and 22. All animals will be sedated with ketamine hydrochloride IM for the ophthalmologic examinations performed following Day 1. For the examinations on Day 1, the animals will be sedated with injectable anesthesia (refer to Section 15.3.3). The eyes will be dilated with 1% tropicamide prior to the examination. The examination will include slit-lamp biomicroscopy and indirect ophthalmoscopy. Additionally, applanation tonometry will be performed on Group 3 animals prior to dosing, immediately following dose administration (-10 to 15 minutes) and on Days 2 and 22.

Blood samples (˜3 mL) will be collected from a peripheral vein for neutralizing antibodies analysis approximately 2 to 3 weeks prior to dose administration.

### 6.25.3. Bioanalytical Sample Collection

Whole blood samples (˜0.5 mL) will be collected from a peripheral vein for bioanalytical analysis (AAV capsid clearance) prior to dose administration, 3 (±10 minutes), 6 (±10 minutes) and 24 (±0.5 hour) hours following dose administration from animals in Group 2 (IV) only. The samples will be collected using a syringe and needle, transferred to two K₂ EDTA tubes and the times recorded.

Blood samples (˜5 mL) will be collected from fasted animals from a peripheral vein for PBMC analysis prior to dose administration (Day 1), on Days 8 and 15 and prior to necropsy (Day 22). The samples will be obtained using lithium heparin tubes and the times recorded.

Blood samples will be collected from a peripheral vein for bioanalytical analysis prior to dose administration (Day 1, 2 mL) and necropsy (Day 22, 5 mL). The samples will be collected in clot tubes and the times recorded. The tubes will be maintained at room temperature until fully clotted, then centrifuged at approximately 2400 rpm at room temperature for 15 minutes. The serum will be harvested, placed in labeled vials (necropsy sample split into 1 mL aliquots), frozen in liquid nitrogen, and stored at −60° C. or below.

CSF (˜1.5 mL) will be collected prior to dose administration from a cisterna magna spinal tap from animals in Group 1 only. CSF (-2 mL) will be collected immediately prior to necropsy from a cisterna magna spinal tap from all animals (Groups 1 to 3). An attempt to collect CSF will be made but due to unsuccessful spinal taps, samples may not be collected at all intervals from an animal(s). Upon collection, the samples will be stored on ice until processing.

### 6.25.4. Necroscopy

A gross necropsy will be performed on any animal found dead or sacrificed moribund, and at the scheduled necropsy, following at least 21 days of treatment (Day 22). All animals, except those found dead, will be sedated with 8 mg/kg of ketamine HCl IM, maintained on an isoflurane/oxygen mixture and provided with an intravenous bolus of heparin sodium, 200 IU/kg. The animals will be perfused via the left cardiac ventricle with 0.001% sodium nitrite in saline. Animals found dead will be necropsied but will not be perfused.

The following tissues will be saved from all animals (including those found dead): Bone marrow, brain, cecum, colon, dorsal nerve roots and ganglion, duodenum, esophagus, eyes with optic nerves, gross lesions, heart, ileum, jejunum, kidneys, knee joint, liver, lungs with bronchi, lymph nodes, ovaries, pancreas, sciatic nerve, skeletal muscle, spinal cord, spleen, thyroids, trachea, and vagus nerve.

### 6.25.5. Bioanalytical Analysis

The whole blood collected from animals in Group 2 (IV) will be evaluated by qPCR and Next-Generation Sequencing (NGS).

PBMC samples collected from all animals will be evaluated by flow cytometry and enzyme-linked immune absorbent spot (ELISpot), if required.

The presence of circulating neutralizing antibodies as well as free vector in the serum and/or CSF will be evaluated by ELISA and cell based assays, as needed.

The vector copy number and number of transcripts in tissues will be examined by quantitative PCR and NGS methods.

### 6.26. Capsid Amino Acid Sequences

Table 10 provides the amino acid sequences of certain engineered capsid proteins described and/or used in studies described herein. Heterologous peptides and amino acid substitutions are indicated in gray shading.

TABLE 10
Capsid Amino Acid Sequences

| Capsid | Insert or | | |
|---|---|---|---|
| Name | Substitution | Amino Acid Sequence | |
| PHP.S | QAVRTSL | 1 MAADGYLPDW LEDNLSEGIR EWWALKPGAP QPKANQQHQD NARGLVLPGY KYLGPGNGLD | |
| (Cali- | (SEQ ID | 61 KGEPVNAADA AALEHDKAYD QQLKAGDNPY LKYNHADAEF QERLKEDTSF GGNLGRAVFQ | |
| fornia | NO: 23) | 121 AKKRLLEPLG LVEEAAKTAP GKKRPVEQSP QEPDSSAGIG KSGAQPAKKR LNFGQTGDTE | |
| Insti- | (588_589) | 181 SVPDPQPIGE PPAAPSGVGS LTMASGGGAP VADNNEGADG VGSSSGNWHC DSQWLGDRVI | |
| tute of | | 241 TTSTRTWALP TYNNHLYKQI SNSTSGGSSN DNAYFGYSTP WGYFDFNRFH CHFSPRDWQR | |
| Tech- | | 301 LINNNWGFRP KRLNFKLFNI QVKEVTDNNG VKTIANNLTS TVQVFTDSDY QLPYVLGSAH | |
| nology | | 361 EGCLPPFPAD VFMIPQYGYL TLNDGSQAVG RSSFYCLEYF PSQMLRTGNN FQFSYEFENV | |
| Chan | | 421 PFHSSYAHSQ SLDRLMNPLI DQYLYYLSRT INGSGQNQQT LKFSVAGPSN MAVQGRNYIP | |
| et al | | 481 GPSYRQQRVS TTVTQNNNSE FAWPGASSWA LNGRNSLMNP GPAMASHKEG EDRFFPLSGS | |
| 2017) | | 541 LIFGKQGTGR DNVDADKVMI TNEEEIKTTN PVATESYGQV 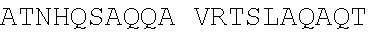 | |
| | | 601 GWVQNQGILP GMVWQDRDVY LQGPIWAKIP HTDGNFHPSP LMGGFGMKHP PPQILIKNTP | |
| | | 661 VPADPPTAFN KDKLNSFITQ YSTGQVSVEI EWELQKENSK RWNPEIQYTS NYYKSNNVEF | |
| | | 721 AVNTEGVYSE PRPIGTRYLT RNL (SEQ ID NO: 60) | |
| PHP.SH | QAVRTSH | 1 MAADGYLPDW LEDNLSEGIR EWWALKPGAP QPKANQQHQD NARGLVLPGY KYLGPGNGLD | |
| | (SEQ ID | 61 KGEPVNAADA AALEHDKAYD QQLKAGDNPY LKYNHADAEF QERLKEDTSF GGNLGRAVFQ | |
| | NO: 24) | 121 AKKRLLEPLG LVEEAAKTAP GKKRPVEQSP QEPDSSAGIG KSGAQPAKKR LNFGQTGDTE | |
| | (588_589) | 181 SVPDPQPIGE PPAAPSGVGS LTMASGGGAP VADNNEGADG VGSSSGNWHC DSQWLGDRVI | |
| | | 241 TTSTRTWALP TYNNHLYKQI SNSTSGGSSN DNAYFGYSTP WGYFDFNRFH CHFSPRDWQR | |
| | | 301 LINNNWGFRP KRLNFKLFNI QVKEVTDNNG VKTIANNLTS TVQVFTDSDY QLPYVLGSAH | |
| | | 361 EGCLPPFPAD VFMIPQYGYL TLNDGSQAVG RSSFYCLEYF PSQMLRTGNN FQFSYEFENV | |
| | | 421 PFHSSYAHSQ SLDRLMNPLI DQYLYYLSRT INGSGQNQQT LKFSVAGPSN MAVQGRNYIP | |
| | | 481 GPSYRQQRVS TTVTQNNNSE FAWPGASSWA LNGRNSLMNP GPAMASHKEG EDRFFPLSGS | |
| | | 541 LIFGKQGTGR DNVDADKVMI TNEEEIKTTN PVATESYGQV 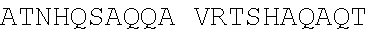 | |
| | | 601 GWVQNQGILP GMVWQDRDVY LQGPIWAKIP HTDGNFHPSP LMGGFGMKHP PPQILIKNTP | |
| | | 661 VPADPPTAFN KDKLNSFITQ YSTGQVSVEI EWELQKENSK RWNPEIQYTS NYYKSNNVEF | |
| | | 721 AVNTEGVYSE PRPIGTRYLT RNL (SEQ ID NO: 61) | |
| PHP.B | TLAVPFK | 1 MAADGYLPDW LEDNLSEGIR EWWALKPGAP QPKANQQHQD NARGLVLPGY KYLGPGNGLD | |
| (Cali- | (SEQ ID | 61 KGEPVNAADA AALEHDKAYD QQLKAGDNPY LKYNHADAEF QERLKEDTSF GGNLGRAVFQ | |
| fornia | NO: 27) | 121 AKKRLLEPLG LVEEAAKTAP GKKRPVEQSP QEPDSSAGIG KSGAQPAKKR LNFGQTGDTE | |
| Insti- | (588_589) | 181 SVPDPQPIGE PPAAPSGVGS LTMASGGGAP VADNNEGADG VGSSSGNWHC DSQWLGDRVI | |
| tute of | | 241 TTSTRTWALP TYNNHLYKQI SNSTSGGSSN DNAYFGYSTP WGYFDFNRFH CHFSPRDWQR | |
| Tech- | | 301 LINNNWGFRP KRLNFKLFNI QVKEVTDNNG VKTIANNLTS TVQVFTDSDY QLPYVLGSAH | |
| nology | | 361 EGCLPPFPAD VFMIPQYGYL TLNDGSQAVG RSSFYCLEYF PSQMLRTGNN FQFSYEFENV | |
| GenBank | | 421 PFHSSYAHSQ SLDRLMNPLI DQYLYYLSRT INGSGQNQQT LKFSVAGPSN MAVQGRNYIP | |
| entry: | | 481 GPSYRQQRVS TTVTQNNNSE FAWPGASSWA LNGRNSLMNP GPAMASHKEG EDRFFPLSGS | |
| ALU851 | | 541 LIFGKQGTGR DNVDADKVMI TNEEEIKTTN PVATESYGQV 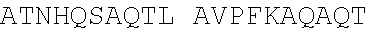 | |
| 56.1- | | 601 GWVQNQGILP GMVWQDRDVY LQGPIWAKIP HTDGNFHPSP LMGGFGMKHP PPQILIKNTP | |
| Deverman | | 661 VPADPPTAFN KDKLNSFITQ YSTGQVSVEI EWELQKENSK RWNPEIQYTS NYYKSNNVEF | |
| et al | | 721 AVNTEGVYSE PRPIGTRYLT RNL (SEQ ID NO: 62) | |
| 2016) | | | |
| PHP.eB | TLAVPFK | 1 MAADGYLPDW LEDNLSEGIR EWWALKPGAP QPKANQQHQD NARGLVLPGY KYLGPGNGLD | |
| (Cali- | (SEQ ID | 61 KGEPVNAADA AALEHDKAYD QQLKAGDNPY LKYNHADAEF QERLKEDTSF GGNLGRAVFQ | |
| fornia | NO: 27) | 121 AKKRLLEPLG LVEEAAKTAP GKKRPVEQSP QEPDSSAGIG KSGAQPAKKR LNFGQTGDTE | |
| Insti- | (588_589) | 181 SVPDPQPIGE PPAAPSGVGS LTMASGGGAP VADNNEGADG VGSSSGNWHC DSQWLGDRVI | |
| tute of | | 241 TTSTRTWALP TYNNHLYKQI SNSTSGGSSN DNAYFGYSTP WGYFDFNRFH CHFSPRDWQR | |
| Tech- | | 301 LINNNWGFRP KRLNFKLFNI QVKEVTDNNG VKTIANNLTS TVQVFTDSDY QLPYVLGSAH | |
| nology- | | 361 EGCLPPFPAD VFMIPQYGYL TLNDGSQAVG RSSFYCLEYF PSQMLRTGNN FQFSYEFENV | |
| Chan | | 421 PFHSSYAHSQ SLDRLMNPLI DQYLYYLSRT INGSGQNQQT LKFSVAGPSN MAVQGRNYIP | |
| et al | | 481 GPSYRQQRVS TTVTQNNNSE FAWPGASSWA LNGRNSLMNP GPAMASHKEG EDRFFPLSGS | |
| 2017) | | 541 LIFGKQGTGR DNVDADKVMI TNEEEIKTTN PVATESYGQV 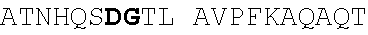 | |
| | | 601 GWVQNQGILP GMVWQDRDVY LQGPIWAKIP HTDGNFHPSP LMGGFGMKHP PPQILIKNTP | |
| | | 661 VPADPPTAFN KDKLNSFITQ YSTGQVSVEI EWELQKENSK RWNPEIQYTS NYYKSNNVEF | |
| | | 721 AVNTEGVYSE PRPIGTRYLT RNL (SEQ ID NO: 63) | |
| AAV8. | A269S | MAADGYLPDW LEDNLSEGIR EWWALKPGAP KPKANQQKQD DGRGLVLPGY KYLGPFNGLD | 60 |
| BBB | | KGEPVNAADA AALEHDKAYD QQLQAGDNPY LRYNHADAEF QERLQEDTSF GGNLGRAVFQ | 120 |
| | | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ 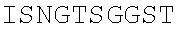 NDNTYFGYST PWGYFDFNRF HCHFSPRDWQ | 300 |
| | | RLINNNWGFR PKRLSFKLFN IQVKEVTQNE GTKTIANNLT STIQVFTDSE YQLPYVLGSA | 360 |
| | | HQGCLPPFPA DVFMIPQYGY LTLNNGSQAV GRSSFYCLEY FPSQMLRTGN NFQFTYTFED | 420 |
| | | VPFHSSYAHS QSLDRLMNPL IDQYLYYLSR TQTTGGTANT QTLGFSQGGP NTMANQAKNW | 480 |
| | | LPGPCYRQQR VSTTTGQNNN SNFAWTAGTK YHLNGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NO: 64) | |
| AAV8. | A269S, | MAADGYLPDW LEDNLSEGIR EWWALKPGAP KPKANQQKQD DGRGLVLPGY KYLGPFNGLD | 60 |
| BBB. | 498_NNN/ | KGEPVNAADA AALEHDKAYD QQLQAGDNPY LRYNHADAEF QERLQEDTSF GGNLGRAVFQ | 120 |
| LD | AAA_500 | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ 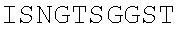 NDNTYFGYST PWGYFDFNRF HCHFSPRDWQ | 300 |
| | | RLINNNWGFR PKRLSFKLFN IQVKEVTQNE GTKTIANNLT STIQVFTDSE YQLPYVLGSA | 360 |
| | | HQGCLPPFPA DVFMIPQYGY LTLNNGSQAV GRSSFYCLEY FPSQMLRTGN NFQFTYTFED | 420 |
| | | VPFHSSYAHS QSLDRLMNPL IDQYLYYLSR TQTTGGTANT QTLGFSQGGP NTMANQAKNW | 480 |
| | | LPGPCYRQQR 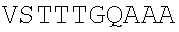 SNFAWTAGTK YHLNGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NO: 65) | |
| AAV9. | S263G/ | MAADGYLPDW LEDNLSEGIR EWWALKPGAP QPKANQQHQD NARGLVLPGY KYLGPGNGLD | 60 |
| BBB | S269T | KGEPVNAADA AALEHDKAYD QQLKAGDNPY LKYNHADAEF QERLKEDTSF GGNLGRAVFQ | 120 |
| | A273T | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI 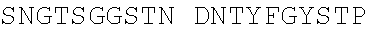 WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 66) | |
| AAV9. | S263G/ | MAADGYLPDW LEDNLSEGIR EWWALKPGAP QPKANQQHQD NARGLVLPGY KYLGPGNGLD | 60 |
| BBB. | S269T | KGEPVNAADA AALEHDKAYD QQLKAGDNPY LKYNHADAEF QERLKEDTSF GGNLGRAVFQ | 120 |
| LD | A273T, | AKKRLLEPLG LVEEAAKTAP GKKRPVEQSP QEPDSSAGIG KSGAQPAKKR LNFGQTGDTE | 180 |
| | 496_NNN/ | SVPDPQPIGE PPAAPSGVGS LTMASGGGAP VADNNEGADG VGSSSGNWHC DSQWLGDRVI | 240 |
| | AAA_498 | TTSTRTWALP TYNNHLYKQI 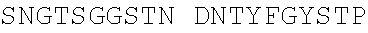 WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS 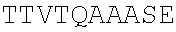 FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 67) | |
| AAVrh. | 498_NNN/ | MAADGYLPDW LEDNLSEGIR EWWDLKPGAP KPKANQQKQD DGRGLVLPGY | 50 |
| 10.LD | AAA_500 | KYLGPFNGLD KGEPVNAADA AALEHDKAYD QQLKAGDNPY LRYNHADAEF | 100 |
| | | QERLQEDTSF GGNLGRAVFQ AKKRVLEPLG LVEEGAKTAP GKKRPVEPSP | 150 |
| | | QRSPDSSTGI GKKGQQPAKK RLNFGQTGDS ESVPDPQPIG EPPAGPSGLG | 200 |
| | | SGTMAAGGGA PMADNNEGAD GVGSSSGNWH CDSTWLGDRV ITTSTRTWAL | 250 |
| | | PTYNNHLYKQ ISNGTSGGST NDNTYFGYST PWGYFDFNRF HCHFSPRDWQ | 300 |
| | | RLINNNWGFR PKRLNFKLFN IQVKEVTQNE GTKTIANNLT STIQVFTDSE | 350 |
| | | YQLPYVLGSA HQGCLPPFPA DVFMIPQYGY LTLNNGSQAV GRSSFYCLEY | 400 |
| | | FPSQMLRTGN NFEFSYQFED VPFHSSYAHS QSLDRLMNPL IDQYLYYLSR | 450 |
| | | TQSTGGTAGT QQLLFSQAGP NNMSAQAKNW LPGPCYRQQR 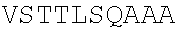 | 500 |
| | | SNFAWTGATK YHLNGRDSLV NPGVAMATHK DDEERFFPSS GVLMFGKQGA | 550 |
| | | GKDNVDYSSV MLTSEEEIKT TNPVATEQYG VVADNLQQQN AAPIVGAVNS | 600 |
| | | QGALPGMVWQ NRDVYLQGPI WAKIPHTDGN FHPSPLMGGF GLKHPPPQIL | 650 |
| | | IKNTPVPADP PTTFSQAKLA SFITQYSTGQ VSVEIEWELQ KENSKRWNPE | 700 |
| | | IQYTSNYYKS TNVDFAVNTD GTYSEPRPIG TRYLTRNL (SEQ ID NO: 68) | |
| AAV9.4 | 498_NNN/ | MAADGYLPDW LEDNLSEGIR EWWALKPGAP QPKANQQHQD NARGLVLPGY KYLGPGNGLD | 60 |
| 96NNN/ | AAA_500 | KGEPVNAADA AALEHDKAYD QQLKAGDNPY LKYNHADAEF QERLKEDTSF GGNLGRAVFQ | 120 |
| AAA498 | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS 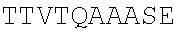 FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 69) | |
| AAV9.4 | 496NNN/ | MAADGYLPDW LEDNLSEGIR EWWALKPGAP QPKANQQHQD NARGLVLPGY KYLGPGNGLD | 60 |
| 96NNN/ | AAA498, | KGEPVNAADA AALEHDKAYD QQLKAGDNPY LKYNHADAEF QERLKEDTSF GGNLGRAVFQ | 120 |
| AAA498. | W503R | AKKRLLEPLG LVEEAAKTAP GKKRPVEQSP QEPDSSAGIG KSGAQPAKKR LNFGQTGDTE | 180 |
| W503R | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS 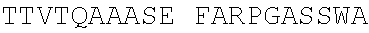 LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 70) | |
| AAV9 | W503R | MAADGYLPDW LEDNLSEGIR EWWALKPGAP QPKANQQHQD NARGLVLPGY KYLGPGNGLD | 60 |
| W503R | | KGEPVNAADA AALEHDKAYD QQLKAGDNPY LKYNHADAEF QERLKEDTSF GGNLGRAVFQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE 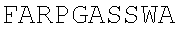 LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 71) | |
| AAV9 | Q474A | MAADGYLPDW LEDNLSEGIR EWWALKPGAP QPKANQQHQD NARGLVLPGY KYLGPGNGLD | 60 |
| Q474A | | KGEPVNAADA AALEHDKAYD QQLKAGDNPY LKYNHADAEF QERLKEDTSF GGNLGRAVFQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSQWLGDRVI | 240 |
| | | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN 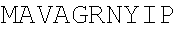 | 480 |
| | | GPSYRQQRVS TTVTQNNNSE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 72) | |
| AAV9 | Bone1, | MAADGYLPDW LEDNLSEGIR EWWALKPGAP QPKANQQHQD NARGLVLPGY KYLGPGNGLD | 60 |
| S454-D8 | DDDDDD | KGEPVNAADA AALEHDKAYD QQLKAGDNPY LKYNHADAEF QERLKEDTSF GGNLGRAVFQ | 120 |
| | DD (SEQ | AKKRLLEPLG LVEEAAKTAP GKKRPVEQSP QEPDSSAGIG KSGAQPAKKR LNFGQTGDTE | 180 |
| | ID NO: 9) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 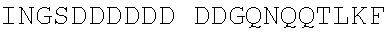 SVAGPSNMAV | 481 |
| | | QGRNYIPGPS YRQQRVSTTV TQNNNSEFAW PGASSWALNG RNSLMNPGPA MASHKEGEDR | 541 |
| | | FFPLSGSLIF GKQGTGRDNV DADKVMITNE EEIKTTNPVA TESYGQVATN HQSAQAQAQT | 601 |
| | | GWVQNQGILP GMVWQDRDVY LQGPIWAKIP HTDGNFHPSP LMGGFGMKHP PPQILIKNTP | 661 |
| | | VPADPPTAFN KDKLNSFITQ YSTGQVSVEI EWELQKENSK RWNPEIQYTS NYYKSNNVEF | 721 |
| | | AVNTEGVYSE PRPIGTRYLT RNL (SEQ ID NO: 73) | |
| AAV9 | Brain1, | MAADGYLPDW LEDNLSEGIR EWWALKPGAP QPKANQQHQD NARGLVLPGY KYLGPGNGLD | 60 |
| S454- | LSSRLDA | KGEPVNAADA AALEHDKAYD QQLKAGDNPY LKYNHADAEF QERLKEDTSF GGNLGRAVFQ | 120 |
| Brain1 | (SEQ ID | AKKRLLEPLG LVEEAAKTAP GKKRPVEQSP QEPDSSAGIG KSGAQPAKKR LNFGQTGDTE | 180 |
| | NO: 10) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 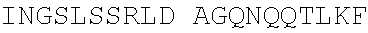 SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 74) | |
| AAV9 | Brain2/ | MAADGYLPDW LEDNLSEGIR EWWALKPGAP QPKANQQHQD NARGLVLPGY KYLGPGNGLD | 60 |
| S454- | Brain1C, | KGEPVNAADA AALEHDKAYD QQLKAGDNPY LKYNHADAEF QERLKEDTSF GGNLGRAVFQ | 120 |
| Brain2 | CLSSRLD | AKKRLLEPLG LVEEAAKTAP GKKRPVEQSP QEPDSSAGIG KSGAQPAKKR LNFGQTGDTE | 180 |
| | AC (SEQ | SVPDPQPIGE PPAAPSGVGS LTMASGGGAP VADNNEGADG VGSSSGNWHC DSQWLGDRVI | 240 |
| | ID NO: | TTSTRTWALP TYNNHLYKQI SNSTSGGSSN DNAYFGYSTP WGYFDFNRFH CHFSPRDWQR | 300 |
| | 11) | LINNNWGFRP KRLNFKLFNI QVKEVTDNNG VKTIANNLTS TVQVFTDSDY QLPYVLGSAH | 360 |
| | (454_455) | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 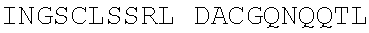 KFSVAGPSNM AV | 482 |
| | | QGRNYIPGPS YRQQRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPQILIKNTP | 662 |
| | | VPADPPTAFN KDKLNSFITQ YSTGQVSVEI EWELQKENSK RWNPEIQYTS NYYKSNNVEF | 722 |
| | | AVNTEGVYSE PRPIGTRYLT RNL (SEQ ID NO: 75) | |
| AAV9 | Kidney 1, | MAADGYLPDW LEDNLSEGIR EWWALKPGAP QPKANQQHQD NARGLVLPGY KYLGPGNGLD | 60 |
| S454- | LPVAS | KGEPVNAADA AALEHDKAYD QQLKAGDNPY LKYNHADAEF QERLKEDTSF GGNLGRAVFQ | 120 |
| Kidney1 | (SEQ ID | AKKRLLEPLG LVEEAAKTAP GKKRPVEQSP QEPDSSAGIG KSGAQPAKKR LNFGQTGDTE | 180 |
| | NO: 13) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 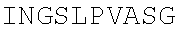 QNQQTLKFSV AGPSNMAV | 478 |
| | | QGRNYIPGPS YRQQRVSTTV TQNNNSEFAW PGASSWALNG RNSLMNPGPA MASHKEGEDR | 538 |
| | | FFPLSGSLIF GKQGTGRDNV DADKVMITNE EEIKTTNPVA TESYGQVATN HQSAQAQAQT | 598 |
| | | GWVQNQGILP GMVWQDRDVY LQGPIWAKIP HTDGNFHPSP LMGGFGMKHP PPQILIKNTP | 658 |
| | | VPADPPTAFN KDKLNSFITQ YSTGQVSVEI EWELQKENSK RWNPEIQYTS NYYKSNNVEF | 718 |
| | | AVNTEGVYSE PRPIGTRYLT RNL (SEQ ID NO: 76) | |
| AAV9 | Kidney2/ | MAADGYLPDW LEDNLSEGIR EWWALKPGAP QPKANQQHQD NARGLVLPGY KYLGPGNGLD | 60 |
| S454- | Kidney1C, | KGEPVNAADA AALEHDKAYD QQLKAGDNPY LKYNHADAEF QERLKEDTSF GGNLGRAVFQ | 120 |
| Kidney2 | CLPVASC | AKKRLLEPLG LVEEAAKTAP GKKRPVEQSP QEPDSSAGIG KSGAQPAKKR LNFGQTGDTE | 180 |
| | (SEQ ID | SVPDPQPIGE PPAAPSGVGS LTMASGGGAP VADNNEGADG VGSSSGNWHC DSQWLGDRVI | 240 |
| | NO 12) | TTSTRTWALP TYNNHLYKQI SNSTSGGSSN DNAYFGYSTP WGYFDFNRFH CHFSPRDWQR | 300 |
| | (454_455) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 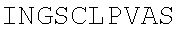 SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 77) | |
| AAV9 | Muscle1, | MAADGYLPDW LEDNLSEGIR EWWALKPGAP QPKANQQHQD NARGLVLPGY KYLGPGNGLD | 60 |
| S454- | ASSLNIA | KGEPVNAADA AALEHDKAYD QQLKAGDNPY LKYNHADAEF QERLKEDTSF GGNLGRAVFQ | 120 |
| Muscle1 | (SEQ ID | AKKRLLEPLG LVEEAAKTAP GKKRPVEQSP QEPDSSAGIG KSGAQPAKKR LNFGQTGDTE | 180 |
| | NO: 14) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 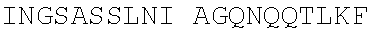 SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 78) | |
| AAV9 | Tfr1 | MAADGYLPDW LEDNLSEGIR EWWALKPGAP QPKANQQHQD NARGLVLPGY KYLGPGNGLD | 60 |
| S454- | HAIYPR | KGEPVNAADA AALEHDKAYD QQLKAGDNPY LKYNHADAEF QERLKEDTSF GGNLGRAVFQ | 120 |
| Tfr1 | (SEQ ID | AKKRLLEPLG LVEEAAKTAP GKKRPVEQSP QEPDSSAGIG KSGAQPAKKR LNFGQTGDTE | 180 |
| | NO: 59) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 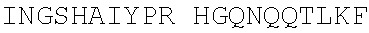 SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 79) | |
| AAV9 | Tfr-3, | MAADGYLPDW LEDNLSEGIR EWWALKPGAP QPKANQQHQD NARGLVLPGY KYLGPGNGLD | 60 |
| S454- | RTIGPSV | KGEPVNAADA AALEHDKAYD QQLKAGDNPY LKYNHADAEF QERLKEDTSF GGNLGRAVFQ | 120 |
| Tfr3 | (SEQ ID | AKKRLLEPLG LVEEAAKTAP GKKRPVEQSP QEPDSSAGIG KSGAQPAKKR LNFGQTGDTE | 180 |
| | NO: 19) | SVPDPQPIGE PPAAPSGVGS LTMASGGGAP VADNNEGADG VGSSSGNWHC DSQWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 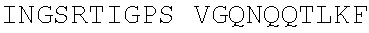 SVAGPSNMAV | 480 |
| | | QGRNYIPGPS YRQQRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPQILIKNTP | 660 |
| | | VPADPPTAFN KDKLNSFITQ YSTGQVSVEI EWELQKENSK RWNPEIQYTS NYYKSNNVEF | 720 |
| | | AVNTEGVYSE PRPIGTRYLT RNL (SEQ ID NO: 80) | |
| AAV9 | Tfr4, | MAADGYLPDW LEDNLSEGIR EWWALKPGAP QPKANQQHQD NARGLVLPGY KYLGPGNGLD | 60 |
| S454- | CRTIGPS | KGEPVNAADA AALEHDKAYD QQLKAGDNPY LKYNHADAEF QERLKEDTSF GGNLGRAVFQ | 120 |
| Tfr4 | VC (SEQ | AKKRLLEPLG LVEEAAKTAP GKKRPVEQSP QEPDSSAGIG KSGAQPAKKR LNFGQTGDTE | 180 |
| (AAV9 | ID NO: | SVPDPQPIGE PPAAPSGVGS LTMASGGGAP VADNNEGADG VGSSSGNWHC DSQWLGDRVI | 240 |
| S454- | 20) | TTSTRTWALP TYNNHLYKQI SNSTSGGSSN DNAYFGYSTP WGYFDFNRFH CHFSPRDWQR | 300 |
| TfR3C) | (454_455) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT 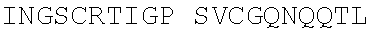 KFSVAGPSNMAV | 482 |
| | | QGRNYIPGPS YRQQRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPQILIKNTP | 662 |
| | | VPADPPTAFN KDKLNSFITQ YSTGQVSVEI EWELQKENSK RWNPEIQYTS NYYKSNNVEF | 722 |
| | | AVNTEGVYSE PRPIGTRYLT RNL (SEQ ID NO: 81) | |
| AAV9.5 | SITLVKST | MAADGYLPDW LEDNLSEGIR EWWALKPGAP QPKANQQHQD NARGLVLPGY KYLGPGNGLD | 60 |
| 88Ad | QTV | KGEPVNAADA AALEHDKAYD QQLKAGDNPY LKYNHADAEF QERLKEDTSF GGNLGRAVFQ | 120 |
| (9 588 | (SEQ ID | AKKRLLEPLG LVEEAAKTAP GKKRPVEQSP QEPDSSAGIG KSGAQPAKKR LNFGQTGDTE | 180 |
| Ad) | NO: 21), | SVPDPQPIGE PPAAPSGVGS LTMASGGGAP VADNNEGADG VGSSSGNWHC DSQWLGDRVI | 240 |
| | DLC-AS1 | TTSTRTWALP TYNNHLYKQI SNSTSGGSSN DNAYFGYSTP WGYFDFNRFH CHFSPRDWQR | 300 |
| | (588_589) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS 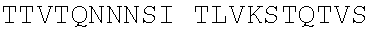 EFAWPGASSW ALNGRNSLMN PGPAMASHKE | 540 |
| | | GEDRFFPLSG SLIFGKQGTG RDNVDADKVM ITNEEEIKTT NPVATESYGQ VATNHQSAQA | 600 |
| | | QAQTGWVQNQ GILPGMVWQD RDVYLQGPIW AKIPHTDGNF HPSPLMGGFG MKHPPPQILI | 660 |
| | | KNTPVPADPP TAFNKDKLNS FITQYSTGQV SVEIEWELQK ENSKRWNPEI QYTSNYYKSN | 720 |
| | | NVEFAVNTEG VYSEPRPIGT RYLTRNL (SEQ ID NO: 82) | |
| AAV9.5 | TILSRSTQ | MAADGYLPDW LEDNLSEGIR EWWALKPGAP QPKANQQHQD NARGLVLPGY KYLGPGNGLD | 60 |
| 88 | TG (SEQ | KGEPVNAADA AALEHDKAYD QQLKAGDNPY LKYNHADAEF QERLKEDTSF GGNLGRAVFQ | 120 |
| Herp | ID NO: | AKKRLLEPLG LVEEAAKTAP GKKRPVEQSP QEPDSSAGIG KSGAQPAKKR LNFGQTGDTE | 180 |
| (9 588 | 22), DLC- | SVPDPQPIGE PPAAPSGVGS LTMASGGGAP VADNNEGADG VGSSSGNWHC DSQWLGDRVI | 240 |
| Hep) | AS2, | TTSTRTWALP TYNNHLYKQI SNSTSGGSSN DNAYFGYSTP WGYFDFNRFH CHFSPRDWQR | 300 |
| | 588_589 | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS 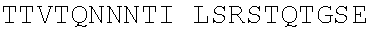 FAWPGASSWA LNGRNSLMNP GPAMASHKEG | 540 |
| | | EDRFFPLSGS LIFGKQGTGR DNVDADKVMI TNEEEIKTTN PVATESYGQV ATNHQSAQAQ | 600 |
| | | AQTGWVQNQG ILPGMVWQDR DVYLQGPIWA KIPHTDGNFH PSPLMGGFGM KHPPPQILIK | 660 |
| | | NTPVPADPPT AFNKDKLNSF ITQYSTGQVS VEIEWELQKE NSKRWNPEIQ YTSNYYKSNN | 720 |
| | | VEFAVNTEG VYSEPRPIGT RYLTRNL (SEQ ID NO: 83) | |
| AAVP | SITLVKST | 1 MAADGYLPDW LEDNLSEGIR EWWALKPGAP QPKANQQHQD NARGLVLPGY KYLGPGNGLD | |
| HPeB.V | QTV | 61 KGEPVNAADA AALEHDKAYD QQLKAGDNPY LKYNHADAEF QERLKEDTSF GGNLGRAVFQ | |
| P2Ad | (SEQ ID | 121 AKKRLLEPLG 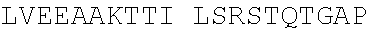 | |
| | NO: 21), | GKKRPVEQSP QEPDSSAGIG KSGAQPAKKR LNFGQTGDTE | |
| | DLC-AS1 | 191 SVPDPQPIGE PPAAPSGVGS LTMASGGGAP VADNNEGADG VGSSSGNWHC DSQWLGDRVI | |
| | (138_139 | 251 TTSTRTWALP TYNNHLYKQI SNSTSGGSSN DNAYFGYSTP WGYFDFNRFH CHFSPRDWQR | |
| | | 311 LINNNWGFRP KRLNFKLFNI QVKEVTDNNG VKTIANNLTS TVQVFTDSDY QLPYVLGSAH | |
| | | 371 EGCLPPFPAD VFMIPQYGYL TLNDGSQAVG RSSFYCLEYF PSQMLRTGNN FQFSYEFENV | |
| | | 431 PFHSSYAHSQ SLDRLMNPLI DQYLYYLSRT INGSGQNQQT LKFSVAGPSN MAVQGRNYIP | |
| | | 491 GPSYRQQRVS TTVTQNNNSE FAWPGASSWA LNGRNSLMNP GPAMASHKEG EDRFFPLSGS | |
| | | 551 LIFGKQGTGR DNVDADKVMI TNEEEIKTTN PVATESYGQV 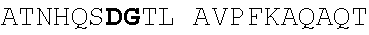 | |
| | | 611 GWVQNQGILP GMVWQDRDVY LQGPIWAKIP HTDGNFHPSP LMGGFGMKHP PPQILIKNTP | |
| | | 671 VPADPPTAFN KDKLNSFITQ YSTGQVSVEI EWELQKENSK RWNPEIQYTS NYYKSNNVEF | |
| | | 731 AVNTEGVYSE PRPIGTRYLT RNL (SEQ ID NO: 84) | |
| AAVP | TILSRSTQ | 1 MAADGYLPDW LEDNLSEGIR EWWALKPGAP QPKANQQHQD NARGLVLPGY KYLGPGNGLD | |
| HPeB.V | TG (SEQ | 61 KGEPVNAADA AALEHDKAYD QQLKAGDNPY LKYNHADAEF QERLKEDTSF GGNLGRAVFQ | |
| P2HerP | ID NO: | 121 AKKRLLEPLG 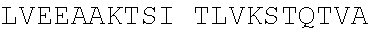 | |
| | 22), DLC- | PGKKRPVEQS PQEPDSSAGI GKSGAQPAKK RLNFGQTGDT E | |
| | AS2, | 192 SVPDPQPIGE PPAAPSGVGS LTMASGGGAP VADNNEGADG VGSSSGNWHC DSQWLGDRVI | |
| | (138_139) | 252 TTSTRTWALP TYNNHLYKQI SNSTSGGSSN DNAYFGYSTP WGYFDFNRFH CHFSPRDWQR | |
| | | 312 LINNNWGFRP KRLNFKLFNI QVKEVTDNNG VKTIANNLTS TVQVFTDSDY QLPYVLGSAH | |
| | | 372 EGCLPPFPAD VFMIPQYGYL TLNDGSQAVG RSSFYCLEYF PSQMLRTGNN FQFSYEFENV | |
| | | 432 PFHSSYAHSQ SLDRLMNPLI DQYLYYLSRT INGSGQNQQT LKFSVAGPSN MAVQGRNYIP | |
| | | 492 GPSYRQQRVS TTVTQNNNSE FAWPGASSWA LNGRNSLMNP GPAMASHKEG EDRFFPLSGS | |
| | | 552 LIFGKQGTGR DNVDADKVMI TNEEEIKTTN PVATESYGQV 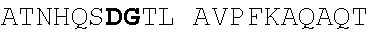 | |
| | | 612 GWVQNQGILP GMVWQDRDVY LQGPIWAKIP HTDGNFHPSP LMGGFGMKHP PPQILIKNTP | |
| | | 672 VPADPPTAFN KDKLNSFITQ YSTGQVSVEI EWELQKENSK RWNPEIQYTS NYYKSNNVEF | |
| | | 732 AVNTEGVYSE PRPIGTRYLT RNL (SEQ ID NO: 85) | |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A recombinant adeno-associated virus (rAAV) capsid protein comprising a peptide insertion of at least 4 and up to 12 contiguous amino acids from a heterologous protein that is not an AAV protein, said peptide insertion being immediately after an amino acid residue corresponding to amino acid 138; or one of amino acids 451 to 461 of AAV9 capsid protein of FIG. 8, wherein said peptide insertion is surface exposed when said capsid protein is packaged as an AAV particle.

2. A recombinant adeno-associated virus (rAAV) capsid protein, said capsid protein comprising a peptide insertion of at least 4 and up to 12 contiguous amino acids from a heterologous protein or domain selected from the group consisting of
(i) a neural tissue-homing protein or domain, with the proviso that the peptide insertion does not comprise sequence TLAVPFK (SEQ ID NO: 27);
(ii) an axonemal or cytoplasmic dynein-homing domain;
(iii) a bone-homing domain;
(iv) a kidney-homing domain;
(v) a muscle-homing domain;
(vi) an endothelial cell-homing domain;
(vii) an integrin receptor-binding domain;
(viii) a transferrin receptor-binding domain, with the proviso that the peptide insertion does not comprise sequence RTIGPSV (SEQ ID NO: 19) nor CRTIGPSVC (SEQ ID NO: 20);
(ix) a tumor cell-targeting domain; or
(x) a retinal cell-homing protein or domain, with the proviso that the peptide insertion does not comprise sequence LGETTRP (SEQ ID NO: 15) nor LALGETTRP (SEQ ID NO: 16);
wherein said peptide insertion is surface exposed when said capsid protein is packaged as an AAV particle.

3. The rAAV capsid protein of claim 1 or 2, wherein said capsid protein is from at least one AAV type selected from AAV type 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype hu.37 (AVVhu.37), serotype rh39 (AAVrh39), and serotype rh74 (AAVrh74).

4. The rAAV capsid protein of any of claims 1-3, wherein said peptide insertion occurs immediately after one of the amino acid residues within:
(a) 450-459 of AAV1 capsid amino acid sequence (SEQ ID NO. 110);
(b) 449-458 of AAV2 capsid amino acid sequence (SEQ ID NO. 111);
(c) 449-459 of AAV3 capsid amino acid sequence (SEQ ID NO. 112);
(d) 443-453 of AAV4 capsid amino acid sequence (SEQ ID NO. 113);
(e) 442-445 of AAV5 capsid amino acid sequence (SEQ ID NO. 114);
(f) 450-459 of AAV6 capsid amino acid sequence (SEQ ID NO. 115);
(g) 451-461 of AAV7 capsid amino acid sequence (SEQ ID NO. 116);
(h) 451-461 of AAV8 capsid amino acid sequence (SEQ ID NO. 117);
(i) 451-461 of AAV9 capsid amino acid sequence (SEQ ID NO. 118);
(j) 452-461 of AAV9e capsid amino acid sequence (SEQ ID NO. 119);
(k) 452-461 of AAVrh10 capsid amino acid sequence (SEQ ID NO. 120);
(l) 452-461 of AAVrh20 capsid amino acid sequence (SEQ ID NO. 121);
(m) 452-461 of AAVhu.37 capsid amino acid sequence (SEQ ID NO. 122);
(n) 452-461 of AAVrh74 capsid amino acid sequence (SEQ ID NO. 123 or SEQ ID NO: 154); or
(o) 452-461 of AAVrh39 capsid amino acid sequence (SEQ ID NO. 124); in the sequences depicted in FIG. 8.

5. The rAAV capsid protein of claim 4, wherein said peptide insertion occurs after an amino acid residue corresponding to one of amino acids I451, N452, G453, S454, G455, Q456, N457, Q458, Q459, T460, or L461 of the AAV9 capsid.

6. The rAAV capsid protein of any of claims 1, 3-5, wherein said heterologous protein is a homing domain, a neutralizing antibody epitope, or a purification tag.

7. The rAAV capsid protein of claim 6, wherein said homing domain is
(i) a neural tissue-homing domain;
(ii) an axonemal or cytoplasmic dynein-homing domain;
(iii) a bone-homing domain;
(iv) a kidney-homing domain;
(v) a muscle-homing domain;
(vi) an endothelial cell-homing domain;
(vii) an integrin receptor-binding domain;
(viii) a transferrin receptor-binding domain;
(ix) a tumor cell-targeting domain; or
(x) a retinal cell-homing domain.

8. The rAAV capsid protein of claim 7, wherein the peptide insertion comprises or consists of at least 4 contiguous amino acids or is 7 contiguous amino acids of a dynein peptide of amino acid sequence SITLVKSTQTV (SEQ ID NO: 21), TILSRSTQTG (SEQ ID NO: 22), VVMVGEKPITITQHSVETEG (SEQ ID NO: 25), RSSEEDKSTQTT (SEQ ID NO: 26), KMQVPFQ (SEQ ID NO: 1), LKLPPIV (SEQ ID NO: 5), PFIKPFE (SEQ ID NO: 6), TLSLPWK (SEQ ID NO: 7), QQAAPSF (SEQ ID NO: 3), RYNAPFK (SEQ ID NO: 4), TLAVPFK (SEQ ID NO: 27), or TLAAPFK (SEQ ID NO: 2).

9. The rAAV capsid protein of claim 7, wherein the peptide insertion from said transferrin receptor-binding domain is at least 4 contiguous amino acids or is 7 amino acids of the amino acid sequence RTIGPSV (SEQ ID NO: 19) or CRTIGPSVC (SEQ ID NO: 20).

10. The rAAV capsid protein of claim 7, wherein the peptide insertion from said retinal cell-homing domain is at least 4 contiguous amino acids or is 7 amino acids of the amino acid sequence TLAAPFK (SEQ ID NO: 2), LGETTRP (SEQ ID NO: 15) or LALGETTRP (SEQ ID NO: 16).

11. The rAAV capsid protein of claim 10, wherein the AAV capsid protein is an AAV8 capsid protein or an AAV9 capsid protein.

12. The rAAV capsid protein of claim 7, wherein the peptide insertion from said purification tag is at least 4 contiguous amino acids or is 7 contiguous amino acids of a hemagglutinin (HA) epitope of amino acid sequence YPYDVPDYA (SEQ ID NO: 86) or of a FLAG tag of amino acid sequence DYKDDDDK (SEQ ID NO: 52).

13. The rAAV capsid protein of claim 2 or 3, wherein said neural tissue-homing protein or said retinal cell-homing protein is a human axonemal dynein (HAD) heavy chain tail.

14. The rAAV capsid protein of claim 13, wherein said peptide insertion comprises at least 4 and up to 15 contiguous amino acids from a dimerization domain of said HAD heavy chain tail.

15. The rAAV capsid protein of claim 14, wherein said peptide insertion comprises at least 4 and up to 15 contiguous amino acids from the group consisting of (depicted in FIG. 7):
(a) (aa 1-1542 of DYH1_HUMAN UniProtKB-Q9P2D7) (SEQ ID NO. 97);
(b) (aa 1-1764 of DYH2_HUMAN UniProtKB-Q9P225) (SEQ ID NO. 98);
(c) (aa 1-1390 of DYH3_HUMAN UniProtKB-Q8TD57) (SEQ ID NO. 99);
(d) (aa 1-1941 of DYH5_HUMAN UniProtKB-Q8TE73) (SEQ ID NO. 100);
(e) (aa 1-1433 of DYH6_HUMAN UniProtKB-Q9COG6) (SEQ ID NO. 101);
(f) (aa 1-1289 of DYH7_HUMAN UniProtKB-Q8WXX0) (SEQ ID NO. 102);
(g) (aa 1-1807 of DYH8_HUMAN UniProtKB-Q96JB1) (SEQ ID NO. 103);
(h) (aa 1-1831 of DYH9_HUMAN UniProtKB-Q9NYC9) (SEQ ID NO. 104);
(i) (aa 1-1793 of DYH10_HUMAN UniProtKB-Q8IVF4) (SEQ ID NO. 105);
(j) (aa 1-1854 of DYH11_HUMAN UniProtKB-Q96DT5) (SEQ ID NO. 106);
(k) (aa 1-1214 of DYH12_HUMAN UniProtKB-Q6ZR08) (SEQ ID NO. 107);
(1) (aa 1-200 of DYH14_HUMAN UniProtKB-QOVDD8) (SEQ ID NO. 108); or
(m) (aa 1-1794 of DYH17_HUMAN UniProtKB-Q9UFH2) (SEQ ID NO. 109).

16. The rAAV capsid protein of claim 15, wherein said peptide insertion comprises at least 4 and up to 15 contiguous amino acids from residues 1-200 of any one of the dynein heavy chain sequences (FIG. 7).

17. The rAAV capsid protein of claim 15, wherein said peptide insertion comprises 7 contiguous amino acids from any one of the dynein heavy chain sequences of FIG. 7.

18. The rAAV capsid protein of claim 16, wherein said peptide insertion comprises 7 contiguous amino acids from residues 1-200 of any one of the dynein heavy chain sequences (FIG. 7).

19. The rAAV capsid protein of claim 2 or 3, wherein said peptide insertion comprises at least 4 contiguous amino acids of one of the peptides:
(a)
(SEQ ID NO: 1)
KMQVPFQ;
(b)
(SEQ ID NO: 2)
TLAAPFK;
(c)
(SEQ ID NO: 3)
QQAAPSF;
(d)
(SEQ ID NO: 4)
RYNAPFK;
(e)
(SEQ ID NO: 5)
LKLPPIV;
(f)
(SEQ ID NO: 6)
PFIKPFE;
or
(g)
(SEQ ID NO: 7)
TLSLPWK.

20. The rAAV capsid protein of claim 2 or 3, wherein said peptide insertion consists of one of the peptides:
(a)
(SEQ ID NO: 1)
KMQVPFQ;
(b)
(SEQ ID NO: 2)
TLAAPFK;
(c)
(SEQ ID NO: 3)
QQAAPSF;
(d)
(SEQ ID NO: 4)
RYNAPFK;
(e)
(SEQ ID NO: 5)
LKLPPIV;
(f)
(SEQ ID NO: 6)
PFIKPFE;
or
(g)
(SEQ ID NO: 7)
TLSLPWK.

21. The rAAV capsid protein of claim 20, wherein said peptide insertion is the amino acid sequence TLAAPFK (SEQ ID NO: 2).

22. The rAAV capsid protein of claim 2 or 3, wherein said neural tissue-homing protein is a mouse axonemal dynein (MAD) heavy chain tail.

23. The rAAV capsid protein of claim 2 or 3, wherein said neural tissue-homing domain is an EPO (erythropoietin) domain that binds innate repair receptor and is not erythropoietic, or a conformational analog of said domain.

24. The rAAV capsid protein of claim 23, wherein the peptide insertion is at least 4 and up to 11 contiguous amino acids from QEQLERALNSS (SEQ ID NO: 8).

25. The rAAV capsid protein of claim 24, wherein said peptide insertion has the amino acid sequence QEQLERALNSS (SEQ ID NO: 8).

26. The rAAV capsid protein of claim 2 or 3, wherein said neural tissue-homing protein is a brain-homing domain having an SRL (serine-arginine-lysine) motif

27. The rAAV capsid protein of claim 26, wherein the peptide insertion from said brain-homing domain has at least 4 contiguous amino acids of or is the amino acid sequence LSSRLDA (SEQ ID NO: 10) or CLSSRLDAC (SEQ ID NO: 11).

28. The rAAV capsid protein of claim 2 or 3, wherein said axonemal or cytoplasmic dynein-homing domain is a dynein light chain-homing domain.

29. The rAAV capsid protein of claim 28, wherein the peptide insertion from said dynein light chain-homing domain is at least 4 and up to 12 contiguous amino acids of one of SITLVKSTQTV (SEQ ID NO: 21), TILSRSTQTG (SEQ ID NO: 22), VVMVGEKPITITQHSVETEG (SEQ ID NO: 25), or RSSEEDKSTQTT (SEQ ID NO:
26).

30. The rAAV capsid protein of claim 2 or 3, wherein said bone-homing protein is a hydroxyapatite (HA)-binding domain.

31. The rAAV capsid protein of claim 30, wherein the peptide insertion from said hydroxyapatite (HA)-binding domain is at least 6 amino acid residues of the sequence DDDDDDDD (SEQ ID NO: 9).

32. The rAAV capsid protein of claim 2 or 3, wherein said kidney-homing domain comprises amino acid sequence CLPVASC (SEQ ID NO: 12).

33. The rAAV capsid protein of claim 32, wherein the peptide insertion from said kidney-homing domain peptide is the amino acid sequence LPVAS (SEQ ID NO: 13) or CLPVASC (SEQ ID NO: 12).

34. The rAAV capsid protein of claim 2 or 3, wherein the peptide insertion from said muscle-homing domain comprises or consists of the amino acid sequence ASSLNIA (SEQ ID NO: 14).

35. The rAAV capsid protein of claim 2 or 3, wherein the peptide insertion comprises or consists of amino acid sequence QAVRTSL (SEQ ID NO: 23) or QAVRTSH (SEQ ID NO: 24).

36. The rAAV capsid protein of claim 2 or 3, wherein the peptide insertion from said endothelial cell-homing domain comprises or consists of the amino acid sequence SIGYPLP (SEQ ID NO: 28).

37. The rAAV capsid protein of claim 2 or 3, wherein the peptide insertion from said integrin-binding domain has amino acid sequence CDCRGDCFC (SEQ ID NO: 29).

38. The rAAV capsid protein of claim 2 or 3, wherein said transferrin receptor-binding domain is a transferrin domain, or a conformation analog thereof, or an iron-mimic.

39. The rAAV capsid protein of claim 38, wherein the peptide insertion from said transferrin domain is at least 4 contiguous amino acids or is 7 contiguous amino acids from sequence HAIYPRH (SEQ ID NO: 17) or THRPPMWSPVWP (SEQ ID NO: 18).

40. rAAV capsid protein of claim 39, wherein the peptide insertion comprises or consists of amino acid sequence HAIYPRH (SEQ ID NO: 17) or THRPPMWSPVWP (SEQ ID NO: 18).

41. The rAAV capsid protein of claim 2 or 3, wherein the peptide insertion from said tumor cell-targeting domain comprises or consists or amino acid sequence NGRAHA (SEQ ID NO: 30).

42. The rAAV capsid protein of claim 2 or 3, wherein the peptide insertion of at least 4 contiguous amino acids or is 7 contiguous amino acids from one of TLAAPFK (SEQ ID NO: 2), TLAVPFK (SEQ ID NO: 27), RTIGPSV (SEQ ID NO: 19), CRTIGPSVC (SEQ ID NO: 20), LGETTRP (SEQ ID NO: 15), and LALGETTRP (SEQ ID NO: 16).

43. The rAAV capsid protein of any of claim 2 or 13-42, wherein said peptide insertion occurs immediately after one of the amino acid residues (as depicted in FIG. 8):
(a) 138, 262-272; 450-459; or 585-593 of AAV1 capsid amino acid sequence (SEQ ID NO. 110);
(b) 138, 262-272; 449-458; or 584-592 of AAV2 capsid amino acid sequence (SEQ ID NO. 111);
(c) 138, 262-272; 449-459; or 585-593 of AAV3 capsid amino acid sequence (SEQ ID NO. 112);
(d) 137, 256-262; 443-453; or 583-591 of AAV4 capsid amino acid sequence (SEQ ID NO. 113);
(e) 137, 252-262; 442-445; or 574-582 of AAV5 capsid amino acid sequence (SEQ ID NO. 114);
(f) 138, 262-272; 450-459; 585-593 of AAV6 capsid amino acid sequence (SEQ ID NO. 115);
(g) 138, 263-273; 451-461; 586-594 of AAV7 capsid amino acid sequence (SEQ ID NO. 116);
(h) 138, 263-274; 452-461; 587-595 of AAV8 capsid amino acid sequence (SEQ ID NO. 117);
(i) 138, 262-273; 452-461; 585-593 of AAV9 capsid amino acid sequence (SEQ ID NO. 118);
(j) 138, 262-273; 452-461; 585-593 of AAV9e capsid amino acid sequence (SEQ ID NO. 119);
(k) 138, 263-274; 452-461; 587-595 of AAVrh10 capsid amino acid sequence (SEQ ID NO. 120);
(l) 138, 263-274; 452-461; 587-595 of AAVrh20 capsid amino acid sequence (SEQ ID NO. 121);
(m)138, 263-274; 452-461; 587-595 of AAVhu37 capsid amino acid sequence (SEQ ID NO. 122);
(n) 138, 263-274; 452-461; 587-595 of AAVrh74 capsid amino acid sequence (SEQ ID NO. 123 or SEQ ID NO: 154); or
(o) 138, 263-274; 452-461; 587-595 of AAVrh39 capsid amino acid sequence (SEQ ID NO. 124).

44. The rAAV capsid protein of claim 43 comprising an amino acid sequence TLAAPFK (SEQ ID NO: 2) inserted between amino acid residues 588-589 of the AAV9 capsid or immediately after an amino acid residue corresponding to amino acid 138 of AAV9 capsid (see FIG. 8).

45. The rAAV capsid protein of claim 43 comprising an amino acid sequence TLAAPFK (SEQ ID NO: 2) inserted after one of I451 to L461, 5268 or Q588 of the AAV9 capsid (FIG. 8).

46. The rAAV capsid protein of claim 43 comprising an amino acid sequence QEQLERALNSS (SEQ ID NO: 8) between amino acid residues 588-589 of the AAV9 capsid (FIG. 8).

47. The rAAV capsid protein of claim 43 comprising an amino acid sequence QEQLERALNSS (SEQ ID NO: 8) inserted at one or more positions selected from I451 to L461, or S268 of the AAV9 capsid (FIG. 8).

48. The rAAV capsid protein of claim 43, wherein said peptide insertion comprises the amino acid sequence TLAVPFK (SEQ ID NO: 27) immediately after one of amino acid residues 262-273 of the AAV9 capsid protein.

49. The rAAV capsid protein of claim 43, wherein said peptide insertion comprises the amino acid sequence LGETTRP (SEQ ID NO: 15) between amino acid residues 269 and 270 of the AAV8 capsid protein.

50. The rAAV capsid protein of claim 49, wherein said peptide insertion has the amino acid sequence LALGETTRP (SEQ ID NO: 16).

51. The rAAV capsid protein of claim 43, wherein said peptide insertion comprises the amino acid sequence LGETTRP (SEQ ID NO: 15) between amino acid residues 590 and 591 of the AAV8 capsid protein.

52. The rAAV capsid protein of claim 51, wherein said peptide insertion has the amino acid sequence LALGETTRP (SEQ ID NO: 16).

53. The rAAV capsid protein of claim 43, wherein said peptide insertion comprises the amino acid sequence LGETTRP (SEQ ID NO: 15) immediately after one of amino acid residues 453 and 454 of the AAV8 capsid protein.

54. The rAAV capsid protein of claim 53, wherein said peptide insertion comprises the amino acid sequence LALGETTRP (SEQ ID NO: 16).

55. The rAAV capsid protein of any of claim 2 or 13-43, wherein said capsid is AAV9 and said peptide insertion occurs between amino acid residues 454 to 455 of AAV9 (FIG. 8).

56. The rAAV capsid protein of any of claim 2 or 13-43, wherein said peptide insertion occurs immediately after an acid residue corresponding to one of I451 to L461, S268 or Q588 of the AAV9 capsid (FIG. 8).

57. The rAAV capsid protein of claim 43, wherein said peptide insertion occurs immediately after one of amino acids 451 to 461 of AAV9 capsid protein.

58. The rAAV capsid protein of any of claim 2 or 13-43, wherein said peptide insertion occurs in an AAV capsid eighth variable region (VR-VIII).

59. The rAAV capsid protein of any of the preceeding claims, with the proviso that said capsid protein is not the AAV2 capsid protein.

60. A recombinant AAV capsid protein comprising one or more amino acid substitutions relative to the wild type or unengineered capsid protein, in which the rAAV capsid protein is an AAV8 capsid protein with an A269S amino acid substitution or is an AAV9 capsid protein with S263G/S269R/A273T substitutions, or W503R or Q474A substitutions, or corresponding substitutions in a capsid protein of another AAV type capsid.

61. The rAAV capsid protein of embodiment 60 further comprising 498-NNN/AAA-500 for an AAV8 capsid protein or 496-NNN/AAA-498 for an AAV9 capsid protein, or corresponding substitutions in a capsid protein of another AAV type capsid.

62. A nucleic acid comprising a nucleotide sequence encoding the rAAV capsid protein of any of the preceding claims, or encoding an amino acid sequence sharing at least 80% identity therewith.

63. The nucleic acid of claim 62 which encodes the rAAV capsid protein of any of the preceding claims.

64. A packaging cell capable of expressing the nucleic acid of claim 62 or 63 to produce AAV vectors comprising the capsid protein encoded by said nucleotide sequence.

65. A rAAV vector comprising the capsid protein of any of claims 1-61.

66. The rAAV vector of claim 65, further comprising a transgene.

67. A pharmaceutical composition comprising the rAAV vector of claim 65 or 66 and a pharmaceutically acceptable carrier.

68. A method of delivering a transgene to a cell, said method comprising contacting said cell with the rAAV vector of claim 65 or 66; or the rAAV vector of claim 65 or 66 for use in delivering a transgene to a cell, wherein said cell is contacted with the vector.

69. A method of delivering a transgene to a target tissue of a subject in need thereof, said method comprising administering to said subject the rAAV vector of claim 65 or 66, wherein said peptide insertion is a homing peptide; or the rAAV vector of claim 65 or 66 for use in delivering a transgene to a target tissue of a subject in need thereof, wherein the vector is administered to said subject.

70. The method, or rAAV vector for use, according to claim 69, wherein said rAAV vector is administered systemically, intravenously, intrathecally, intra-nasally, intra-peritoneally, intravitreally, via lumbar puncture or via the cisterna magna.

71. The method, or rAAV vector for use, according to claim 70, wherein said target tissue is:
(i) a neural tissue, and said vector comprises the peptide insertion from said neural tissue-homing domain;
(ii) bone, and said vector comprises the peptide insertion from said bone-homing domain;
(iii) kidney, and said vector comprises the peptide insertion from said kidney-homing domain;
(iv) muscle, and said vector comprises the peptide insertion from said muscle-homing domain;
(v) an endothelial cell, and said vector comprises the peptide insertion from said endothelial cell-homing domain;
(vi) an integrin receptor, and said vector comprises the peptide insertion from said integrin receptor-binding domain;
(vii) a transferrin receptor on a tumor cell, and said vector comprises the peptide insertion from said transferrin receptor-binding domain;
(viii) a tumor cell, and said vector comprises the peptide insertion from said tumor cell-targeting domain; or
(ix) a retinal cell, and said vector comprises the peptide insertion from said retinal cell-homing domain.

72. The method, or rAAV vector for use, of claim 71, wherein said target tissue is retinal cells and the peptide insertion comprises the amino acid sequence TLAAPFK (SEQ ID NO: 2), LGETTRP (SEQ ID NO: 15) or LALGETTRP (SEQ ID NO: 16).

73. The method, or rAAV vector for use, of claim 72, wherein said target tissue is retinal cells and the peptide insertion comprises the amino acid sequence TLAAPFK (SEQ ID NO: 2).
